(19) United States
(12) Patent Application Publication
(10) Pub. No.: US 2010/0216802 A1
(43) Pub. Date: Aug. 26, 2010

(54) PTERIDINE DERIVATIVES AS POLO-LIKE KINASE INHIBITORS USEFUL IN THE TREATMENT OF CANCER

(75) Inventors: David Festus Charles Moffat, Oxfordshire (GB); Sanjay Ratilal Patel, Oxfordshire (GB); Stephen John Davies, Oxfordshire (GB); Kenneth William John Baker, Oxfordshire (GB); Oliver James Philps, Oxfordshire (GB)

Correspondence Address: BANNER & WITCOFF, LTD., 1100 13th STREET, N.W., SUITE 1200, WASHINGTON, DC, 20005-4051, US

(73) Assignee: CHROMA THERAPEUTICS LTD., Abingdon, Oxfordshire (GB)

(21) Appl. No.: 12/446,008

(22) PCT Filed: Oct. 19, 2007
(86) PCT No.: PCT/GB07/03998

§ 371 Date: Dec. 3, 2009

(30) Foreign Application Priority Data
Oct. 25, 2006 (GB) 0621205.4
Aug. 10, 2007 (GB) 0715614.4

(51) Int. Cl. A61K 31/519; C07D 475/00; A61P 35/00

(52) U.S. Cl. 514/249; 544/258

(57) ABSTRACT

Compound of formula (I) are inhibitors of Polo-like kinases (PLKs), and are useful in treatment of cell proliferative diseases:

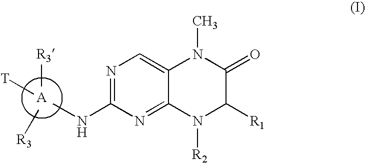

wherein R₁ and R₂ are hydrogen, or an optionally substituted (C₁-C₆)alkyl, (C₂-C₆)alkenyl, (C₂-C₆)alkynyl or (C₃-C₆)cycloalkyl group; R₃ and R₃′ are independently selected from hydrogen, —CN, hydroxyl, halogen, optionally substituted (C₁-C₆)alkyl, (C₂-C₆)alkenyl, (C₂-C₆)alkynyl or (C₃-C₆)cycloalkyl, —NR₅R₆ or C₁-C₄ alkoxy, wherein R₅ and R₆ are independently hydrogen or optionally substituted (C₁-C₆)alkyl; ring A is an optionally substituted mono- or bi-cyclic carbocyclic or heterocyclic ring or a ring system having up to 12 ring atoms; T is a radical of formula R-L¹-Y¹— wherein R is an alpha amino acid or alpha amino acid ester motif, linked to ring A by linker R-L¹-Y¹— as defined in the claims.

This invention relates to a series of amino acid esters, to compositions containing them, to processes for their preparation and to their use in medicine as Polo-like kinase ‘PLK’ inhibitors. Polo-like kinases (PLKs) are key enzymes that control mitotic entry of proliferating cells and regulate many aspects of mitosis necessary for successful cytokinesis. Of the four known human PLKs, PLK1 is the best characterized and is overexpressed in many tumour types with aberrant elevation frequently constituting a prognostic indicator of poor disease outcome. The compounds may be of use in the treatment of cell proliferative diseases such as cancer. The present invention encompasses compounds that are dihydropteridinine derivatives.

### BACKGROUND TO INVENTION

The PLKs, a family of Ser/Thr protein kinases named after their functional and sequence similarity with the archetypal polo kinase from Drosophila melanogaster, play a variety of roles in mitosis (Nat. Rev. Mol. Cell. Biol., 2001, 2, 21-32.). In yeasts (Saccharomyces cerevisiae and S. pombe) single PLKs exist, whereas four distinct PLKs have been identified to date in mammals. Human PLK1 (Cell Growth Differ., 1994, 5, 249-257), PLK2 (serum-inducible kinase, SNK, Mol. Cell. Biol., 1992, 12, 4164-4169), PLK3 (proliferation-related kinase, PRK J. Biol. Chem. 1997, 272, 28646-28651) and PLK4 (Oncol. Rep., 1997, 4, 505-510) are structurally homologous and contain two conserved domains, the N-terminal catalytic kinase domain, as well as a C-terminal region composed of the so-called polo boxes. Whereas PLK1, PLK2, and PLK3 are expressed in all tissues, PLK4 appears to possess unique physiological roles and the distribution of PLK4 mRNA in adults is restricted to certain tissues such as testes and thymus. PLK1 is the best characterized member of the PLK family and it appears to fulfil most of the known functions of the single PLKs present in invertebrates (Nat. Rev. Mol. Cell. Biol., 2004, 5, 429-441). PLK1 protein levels fluctuate in a cell-cycle-dependent manner and its kinase activity peaks at the transition between the second gap phase and the mitosis phases (G2/M) of the eukaryotic cell division cycle. Upon exit from mitosis PLK1 levels drop as a result of ubiquitin-dependent proteolysis. PLK1 has been reported to be involved in the initiation of mitosis through activation of the cyclin-dependent kinase CDK1/cyclin B complex, i.e. the master switch for mitotic entry (mitosis-promoting factor, MPF Nature, 1990, 344, 503-508).

This occurs when PLK1 phosphorylates, and thus activates, the dual specificity phosphatase CDC25C, which in turn relieves premitotic MYT1- and WEE1-mediated suppression of CDK1/cyclin B activity through dephosphorylation at the CDK1 pThr14 and pTyr15 sites (Cell, 1991, 67, 197-211). Upon entry into mitosis, phosphorylation of CDC25C by PLK1 and PLK3 leads to its translocation into the nucleus. Apart from controlling entry into mitosis through CDK1 activation, PLK1 has additional roles in regulating progression through mitosis. It is involved in bipolar spindle formation, including centrosome maturation and regulation of the microtubule organizing centre, in the subsequent steps of mitosis involving sister chromatid separation, and finally in cytokinesis (Dev. Cell, 2003, 5, 127-138).

### BRIEF SUMMARY OF THE INVENTION

Compounds of the invention are related to compounds disclosed in WO2004076454. They are inhibitors of PLK1 and the isoforms thereof. The compounds are thus of use in medicine, for example in the treatment of a variety of proliferative disease states, including cancers. The compounds are characterised by the presence in the molecule of an amino acid motif or an amino acid ester motif which is hydrolysable by an intracellular carboxylesterase. Compounds of the invention having the lipophilic amino acid ester motif cross the cell membrane, and are hydrolysed to the acid by the intracellular carboxylesterases. The polar hydrolysis product accumulates in the cell since it does not readily cross the cell membrane. Hence the PLK1 activity of the compound is prolonged and enhanced within the cell.

### DETAILED DESCRIPTION OF THE INVENTION

According to the invention there is provided a compound of formula (I), or a salt, N-oxide, hydrate or solvate thereof:

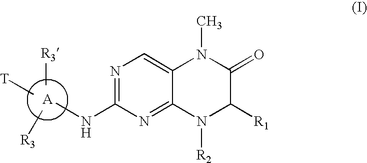

wherein
R₁ is hydrogen, or an optionally substituted (C₁-C₆)alkyl, (C₂-C₆)alkenyl, (C₂-C₆)alkynyl or (C₃-C₆)cycloalkyl group;
R₂ is hydrogen, or an optionally substituted (C₁-C₆)alkyl, (C₂-C₆)alkenyl, (C₂-C₆)alkynyl or (C₃-C₆)cycloalkyl group;
R₃ and R₃′ are independently selected from hydrogen, —CN, hydroxyl, halogen, optionally substituted (C₁-C₆)alkyl, (C₂-C₆)alkenyl, (C₂-C₆)alkynyl or (C₃-C₆)cycloalkyl, —NR₅R₆ or C₁-C₄ alkoxy, wherein R₅ and R₆ are independently hydrogen or optionally substituted (C₁-C₆)alkyl;
ring A is an optionally substituted mono- or bi-cyclic carbocyclic or heterocyclic ring or a ring system having up to 12 ring atoms;
T is a radical of formula R-L¹-Y¹— wherein
Y¹ is a bond, —O—, —S—, —NR₆—, —(C═O)—, —S(O₂)—, —(C═O)NR₆—, —NR₆(C═O)—, —S(O₂)NR₆—, —NR₆S(O₂)—, or —NR₆(C═O)NR₉—, wherein R₆ and R₉ are independently hydrogen or optionally substituted (C₁-C₆)alkyl;
L¹ is a divalent radical of formula -(Alk¹)ₘ(Q)ₙ(Alk²)ₚ- wherein
m, n and p are independently 0 or 1,
Q is (i) an optionally substituted divalent mono- or bicyclic carbocyclic or heterocyclic radical having 5-13 ring members, or (ii), in the case where p is 0, a divalent radical of formula -Q¹-X²— wherein X² is —O—, —S— or NR^{A}— wherein R^{A} is hydrogen or optionally substituted C₁-C₃ alkyl, and Q¹ is an optionally substituted divalent mono- or bicyclic carbocyclic or heterocyclic radical having 5-13 ring members,
Alk¹ and Alk² independently represent optionally substituted divalent C₃-C₇ cycloalkyl radicals, or optionally substituted straight or branched, C₁-C₆ alkylene, C₂-C₆ alkenylene, or C₂-C₆ alkynylene radicals which may optionally contain or terminate in an ether (—O—), thioether (—S—) or amino (—NR^{A}—) link wherein R^{A} is hydrogen or optionally substituted C₁-C₃ alkyl;
   R is a radical of formula (X) or (Y)

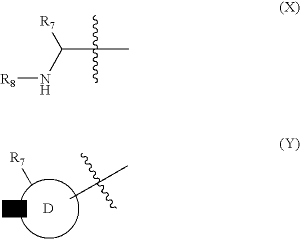

wherein
R₇ is a carboxylic acid group (—COON), or an ester group which is hydrolysable by one or more intracellular carboxylesterase enzymes to a carboxylic acid group;
R₈ is hydrogen; or optionally substituted C₁-C₆ alkyl, C₃-C₇ cycloalkyl, aryl or heteroaryl or —(C═O)R₆, —(C═O)OR₆, or —(C═O)NR₆ wherein R₆ is hydrogen or optionally substituted (C₁-C₆)alkyl; and
D is a monocyclic heterocyclic ring of 5 or 6 ring atoms wherein R₇ is linked to a ring carbon adjacent to the ring nitrogen shown, and ring D is optionally fused to a second carbocyclic or heterocyclic ring of 5 or 6 ring atoms in which case the bond shown intersected by a wavy line may be from a ring atom in said second ring.

In the compounds of the invention, when R₁ is other than hydrogen, the carbon atom to which the R₁ substituent is attached is asymmetric. Preferably the stereo chemistry at that asymmetric center is R.

In another broad aspect the invention provides the use of a compound of formula (I) as defined above, or an N-oxide, salt, hydrate or solvate thereof in the preparation of a composition for inhibiting the activity of PLK1.

The compounds with which the invention is concerned may be used for the inhibition of PLK1 activity ex vivo or in vivo.

In one aspect of the invention, the compounds of the invention may be used in the preparation of a composition for treatment of cell proliferative diseases such as solid tumours and haemato-oncological tumours such as leukaemias and lymphomas.

In another aspect, the invention provides a method for the treatment of the foregoing disease types, which comprises administering to a subject suffering such disease an effective amount of a compound of formula (I) as defined above.

### Terminology

As used herein, the term “(Cₐ-C_{b})alkyl” wherein a and b are integers, refers to a straight or branched chain alkyl radical having from a to b carbon atoms. Thus when a is 1 and b is 6, for example, the term includes methyl, ethyl, n-propyl, isopropyl, n-butyl, isobutyl, sec-butyl, t-butyl, n-pentyl and n-hexyl.

As used herein, the term “divalent (Cₐ-C_{b})alkylene radical”, wherein a and b are integers, refers to a saturated hydrocarbon chain having from a to b carbon atoms and two unsatisfied valences.

As used herein, the term “(Cₐ-C_{b})alkenyl” wherein a and b are integers, refers to a straight or branched chain alkenyl moiety with a to b carbon atoms; having at least one double bond of either E or Z stereochemistry where applicable. The term includes, for example, vinyl, allyl, 1- and 2-butenyl and 2-methyl-2-propenyl.

As used herein, the term “divalent (Cₐ-C_{b})alkenylene radical” means a hydrocarbon chain having from a to b carbon atoms, at least one double bond, and two unsatisfied valences.

As used herein the term “Cₐ-C_{b} alkynyl”, wherein a and b are integers refers to straight chain or branched chain hydrocarbon groups having from two to six carbon atoms and having in addition one triple bond. This term would include, for example, ethynyl, 1-propynyl, 1- and 2-butynyl, 2-methyl-2-propynyl, 2-pentynyl, 3-pentynyl, 4-pentynyl, 2-hexynyl, 3-hexynyl, 4-hexynyl and 5-hexynyl.

As used herein, the term “divalent (Cₐ-C_{b})alkynylene radical”, wherein a and b are integers refers to a divalent hydrocarbon chain having from two to six carbon atoms, and at least one triple bond.

As used herein, the term “carbocyclic” refers to a mono-, bi- or tricyclic radical having up to 16 ring atoms, all of which are carbon, and includes aryl and cycloalkyl.

As used herein, the term “cycloalkyl” refers to a monocyclic saturated carbocyclic radical having from 3-8 carbon atoms and includes, for example, cyclopropyl, cyclobutyl, cyclopentyl, cyclohexyl, cycloheptyl and cyclooctyl.

As used herein, the unqualified term “aryl” refers to a mono-, bi- or tri-cyclic carbocyclic aromatic radical, and includes radicals having two monocyclic carbocyclic aromatic rings which are directly linked by a covalent bond. Illustrative of such radicals are phenyl, biphenyl and napthyl.

As used herein, the unqualified term “heteroaryl” refers to a mono-, bi- or tri-cyclic aromatic radical containing one or more heteroatoms selected from S, N and O, and includes radicals having two such monocyclic rings, or one such monocyclic ring and one monocyclic aryl ring, which are directly linked by a covalent bond. Illustrative of such radicals are thienyl, benzthienyl, furyl, benzfuryl, pyrrolyl, imidazolyl, benzimidazolyl, thiazolyl, benzthiazolyl, isothiazolyl, benzisothiazolyl, pyrazolyl, oxazolyl, benzoxazolyl, isoxazolyl, benzisoxazolyl, isothiazolyl, triazolyl, benztriazolyl, thiadiazolyl, oxadiazolyl, pyridinyl, pyridazinyl, pyrimidinyl, pyrazinyl, triazinyl, indolyl and indazolyl.

As used herein, the unqualified term “heterocyclyl” or “heterocyclic” includes “heteroaryl” as defined above, and in its non-aromatic meaning relates to a mono-, bi- or tri-cyclic non-aromatic radical containing one or more heteroatoms selected from S, N and O, and to groups consisting of a monocyclic non-aromatic radical containing one or more such heteroatoms which is covalently linked to another such radical or to a monocyclic carbocyclic radical. Illustrative of such radicals are pyrrolyl, furanyl, thienyl, piperidinyl, imidazolyl, oxazolyl, isoxazolyl, thiazolyl, thiadiazolyl, pyrazolyl, pyridinyl, pyrrolidinyl, pyrimidinyl, morpholinyl, piperazinyl, indolyl, morpholinyl, benzfuranyl, pyranyl, isoxazolyl, benzimidazolyl, methylenedioxyphenyl, ethylenedioxyphenyl, maleimido and succinimido groups.

A “divalent phenylene, pyridinylene, pyrimidinylene, pyrazinylene, piperidinylene, piperazinylene, pyrrolidenylene, pyrrolene, cyclopropylene, cyclobutylene, cyclopentylene, cyclohexylene or 3-aza-bicyclo[3.1.0]hexylene, radical” is a benzene, pyridine, pyrimidine, pyrazine, piperidine, piperazine, pyrrolidene, pyrrole, cyclopropyl, cyclobutylene, cyclopentyl, cyclohexyl or 3-aza-bicyclo[3.1.0]hexyl ring, with two unsatisfied valencies, and includes 1,3-phenylene, 1,4-phenylene, and the following:

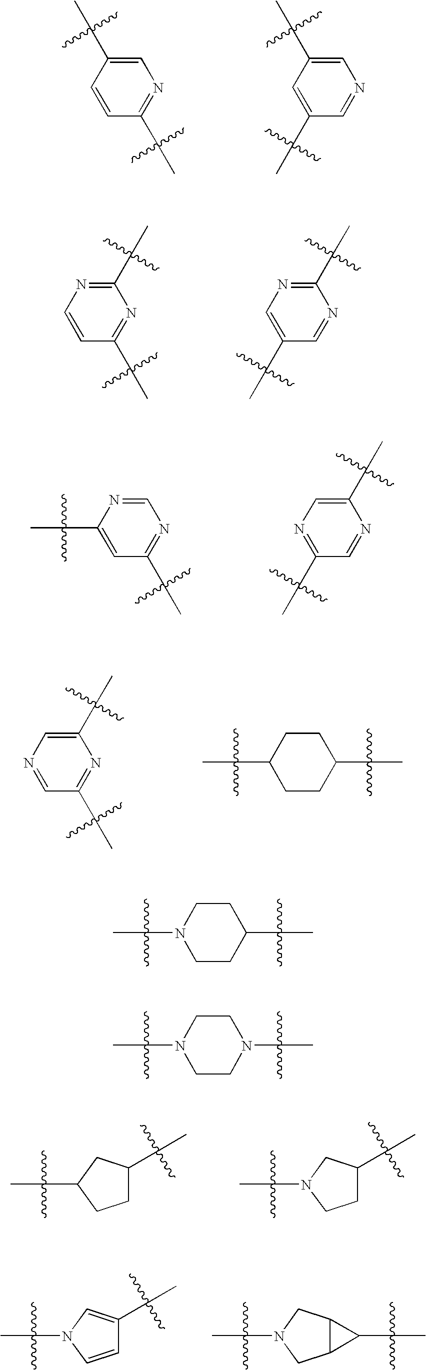

Unless otherwise specified in the context in which it occurs, the term “substituted”, as applied to any moiety herein, means substituted with up to four compatible substituents, each of which independently may be, for example, (C₁-C₆)alkyl, (C₁-C₆)alkoxy, hydroxy, hydroxy(C₁-C₆)alkyl, mercapto, mercapto(C₁-C₆)alkyl, (C₁-C₆)alkylthio, phenyl, halo (including fluoro, bromo and chloro), trifluoromethyl, trifluoromethoxy, nitro, nitrile (—CN), oxo, —COOH, —COOR^{A}, —COR^{A}, —SO₂R^{A}, —CONH₂, —SO₂NH₂, —CONHR^{A}, —SO₂NHR^{A}, —CONR^{A}R^{B}, —SO₂NR^{A}R^{B}, —NH₂, —NHR^{A}, —NR^{A}R^{B}, —OCONH₂, —OCONHR^{A}, —OCONR^{A}R^{B}, —NHCOR^{A}, —NHCOOR^{A}, —NR^{B}COOR^{A}, —NHSO₂OR^{A}, —NR^{B}SO₂OH, —NR^{B}SO₂OR^{A}, —NHCONH₂, —NR^{A}CONH₂, —NHCONHR^{B}, —NR^{A}CONHR^{B}, —NHCONR^{A}R^{B}, or —NR^{A}CONR^{A}R^{B} wherein R^{A} and R^{B} are independently a (C₁-C₆)alkyl, (C₃-C₆) cycloalkyl, phenyl or monocyclic heteroaryl having 5 or 6 ring atoms, or R^{A} and R^{B} when attached to the same nitrogen atom form a cyclic amino group (for example morpholino, piperidinyl, piperazinyl, or tetrahydropyrrolyl). An “optional substituent” may be one of the foregoing substituent groups.

As used herein the term “salt” includes base addition, acid addition and quaternary salts. Compounds of the invention which are acidic can form salts, including pharmaceutically acceptable salts, with bases such as alkali metal hydroxides, e.g. sodium and potassium hydroxides; alkaline earth metal hydroxides e.g. calcium, barium and magnesium hydroxides; with organic bases e.g. N-methyl-D-glucamine, choline tris(hydroxymethyl)amino-methane, L-arginine, L-lysine, N-ethyl piperidine, dibenzylamine and the like. Those compounds (I) which are basic can form salts, including pharmaceutically acceptable salts with inorganic acids, e.g. with hydrohalic acids such as hydrochloric or hydrobromic acids, sulphuric acid, nitric acid or phosphoric acid and the like, and with organic acids e.g. with acetic, tartaric, succinic, fumaric, maleic, malic, salicylic, citric, methanesulphonic, p-toluenesulphonic, benzoic, benzenesunfonic, glutamic, lactic, and mandelic acids and the like.

Compounds of the invention which contain one or more actual or potential chiral centres, because of the presence of asymmetric carbon atoms, can exist as a number of diastereoisomers with R or S stereochemistry at each chiral centre. The invention includes all such diastereoisomers and mixtures thereof.

The term “ester” or “esterified carboxyl group” in connection with substituent R₇ above means a group RₓO(C═O)— in which Rₓ is the group characterising the ester, notionally derived from the alcohol RₓOH.

### The Substituents R₁—R₃′

R₁ is hydrogen, (C₁-C₆)alkyl, for example methyl, ethyl, n- or iso-propyl, (C₂-C₆)alkenyl, for example allyl, (C₂-C₆)alkynyl, for example —CH₂C≡CH or (C₃-C₆)cycloalkyl, for example cyclopropyl, cyclopentyl or cyclohexyl. In one subclass of compounds of the invention R₁ is ethyl.

R₂ is hydrogen, (C₁-C₆)alkyl, for example methyl, ethyl, n- or iso-propyl, (C₂-C₆)alkenyl, for example allyl, (C₂-C₆)alkynyl, for example —CH₂C—≡CH or (C₃-C₆)cycloalkyl, for example cyclopropyl, cyclopentyl or cyclohexyl, or C₆₋₁₄ aryl for example phenyl or naphthyl. In one subclass of compounds of the invention R₂ is cyclopentyl.

R₃ and R₃′ are independently selected from hydrogen, —CN, hydroxyl, halogen, (C₁-C₆)alkyl, for example methyl, ethyl, n- or iso-propyl, (C₂-C₆)alkenyl, for example allyl, (C₂-C₆)alkynyl, for example —CH₂C≡CH or (C₃-C₆)cycloalkyl, for example cyclopropyl, cyclopentyl or cyclohexyl, —NR₅R₆ and C₁-C₄ alkoxy, wherein R₅ and R₆ are independently hydrogen or optionally substituted (C₁-C₆)alkyl, for example methyl or ethyl. In one subclass of compounds of the invention R₃ is methoxy, fluoro or chloro, and R′₃ is hydrogen, fluoro or chloro.

### The Ring A

Ring A is a mono- or bi-cyclic carbocyclic or heterocyclic ring or a ring system having up to 12 ring atoms. Examples of such rings are piperidine, piperazine, pyridine, pyrimidine, pyrazoline, triazoline, furan, thiophene, pyrrole, thiazole, isothiazole, oxazole, isoxazole, and thiadiazole rings. Currently preferred rings A are phenyl, pyridinyl and pyrimidinyl.

Ring A may be substituted by any of the optional substituents referred to above, for example chloro, bromo or fluoro, trifluoromethyl, methoxy, and trifluoromethoxy.

### The Substituent T

This substituent contains the alpha amino acid or alpha amino acid ester moiety of formula (X) or (Y), linked through a linker radical to ring A.

The ester compounds of the invention are converted by intracellular esterases to the carboxylic acid. Both the esters and carboxylic acids may have PLK inhibitory activity in their own right. The compounds of the invention therefore include not only the ester, but also the corresponding carboxylic acid hydrolysis products.

The ester group R₇ present in substituent T must be one which in the compound of the invention is hydrolysable by one or more intracellular carboxylesterase enzymes to a carboxylic acid group. Intracellular carboxylesterase enzymes capable of hydrolysing the ester group of a compound of the invention to the corresponding acid include the three known human enzyme isotypes hCE-1, hCE-2 and hCE-3. Although these are considered to be the main enzymes other enzymes such as biphenylhydrolase (BPH) may also have a role in hydrolysing the conjugates. In general, if the carboxylesterase hydrolyses the free amino acid ester to the parent acid it will also hydrolyse the ester motif when covalently conjugated to the modulator. Hence, the broken cell assay described herein provides a straightforward, quick and simple first screen for esters which have the required hydrolysis profile. Ester motifs selected in that way may then be re-assayed in the same carboxylesterase assay when conjugated to the rest of the molecule via the chosen conjugation chemistry, to confirm that it is still a carboxylesterase substrate in that background.

Subject to the requirement that they be hydrolysable by intracellular carboxylesterase enzymes, examples of particular ester groups R₇ include those of formula —(C═O)OR₁₀ wherein R₁₀ is R₁₁R₁₂R₁₃C— wherein
(i) R₁₁ is hydrogen or optionally substituted (C₁-C₃)alkyl-(Z¹)ₐ-[(C₁-C₃)alkyl]_{b}- or (C₂-C₃)alkenyl-(Z¹)ₐ-[(C₁-C₃)alkyl]_{b}- wherein a and b are independently 0 or 1 and Z¹ is —O—, —S—, or —NR₁₄— wherein R₁₄ is hydrogen or (C₁-C₃)alkyl; and R₁₂ and R₁₃ are independently hydrogen or (C₁-C₃)alkyl-;
(ii) R₁₁ is hydrogen or optionally substituted R₁₅R₁₆N—(C₁-C₃)alkyl- wherein R₁₅ is hydrogen or (C₁-C₃)alkyl and R₁₆ is hydrogen or (C₁-C₃)alkyl; or R₁₅ and R₁₆ together with the nitrogen to which they are attached form an optionally substituted monocyclic heterocyclic ring of 5- or 6-ring atoms or bicyclic heterocyclic ring system of 8 to 10 ring atoms, and R₁₂ and R₁₃ are independently hydrogen or (C₁-C₃)alkyl-; or
(iii) R₁₁ and R₁₂ taken together with the carbon to which they are attached form an optionally substituted monocyclic carbocyclic ring of from 3 to 7 ring atoms or bicyclic carbocyclic ring system of 8 to 10 ring atoms, and R₁₃ is hydrogen.

Within these classes, R₁₀ may be, for example, methyl, ethyl, n- or iso-propyl, n-, sec- or tert-butyl, cyclohexyl, allyl, phenyl, benzyl, 2-, 3- or 4-pyridylmethyl, N-methylpiperidin-4-yl, tetrahydrofuran-3-yl, methoxyethyl, indanyl, norbonyl, dimethylaminoethyl, or morpholinoethyl. Currently preferred is where R₁₀ is cyclopentyl or tert-butyl.

### The Ring D

When R is a group of formula (Y), examples of R include:

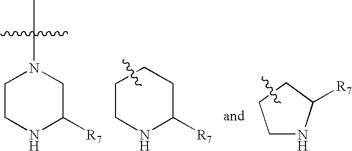

wherein R₇ is as defined and discussed above.

### The Group R₈

The group R₈ is present in the compounds of the invention when R in formula (I) is a radical of formula (X)

R₈ may be, for example, optionally substituted (C₁-C₆)alkyl, (C₃-C₆)cycloalkyl, aryl or heteroaryl, for example methyl, ethyl, n- or isopropyl, cyclopropyl, cyclopentyl, cyclohexyl, phenyl, or pyridyl. R₈ may also be, for example hydrogen or —(C═O)R₁₆, wherein R₁₆ is optionally substituted (C₁-C₆)alkyl such as methyl, ethyl, n- or isopropyl, or n-, iso- or sec-butyl, (C₃-C₆)cycloalkyl such as cyclopropyl, cyclopentyl, cyclohexyl, phenyl, pyridyl, thienyl, phenyl(C₁-C₆alkyl)-, thienyl(C₁-C₆alkyl)- or pyridyl(C₁-C₆alkyl)-such as benzyl, 4-methoxyphenylmethylcarbonyl, thienylmethyl or pyridylmethyl.

R₈ may also be, for example —(C═O)OR₁₇, or —(C═O)NHR₁₇ wherein R₁₇ is hydrogen or optionally substituted (C₁-C₆)alkyl such as methyl, ethyl, or n- or isopropyl.

Currently it is preferred that R₈ be hydrogen.

For compounds of the invention which are to be administered systemically, esters with a slow rate of esterase cleavage are preferred, since they are less susceptible to pre-systemic metabolism. Their ability to reach their target tissue intact is therefore increased, and the ester can be converted inside the cells of the target tissue into the acid product. However, for local administration, where the ester is either directly applied to the target tissue or directed there by, for example, inhalation, it will often be desirable that the ester has a rapid rate of esterase cleavage, to minimise systemic exposure and consequent unwanted side effects. If a carbon atom to which the group R is attached is unsubstituted, ie R is attached to a methylene (—CH₂)— radical, then the esters tend to be cleaved more rapidly than if that carbon is substituted, or is part of a ring system such as a phenyl or cyclohexyl ring.

The radical -L¹-Y¹—

This radical (or bond) arises from the particular chemistry strategy chosen to link the amino acid ester motif R in substituent T to ring A of the inhibitor. Clearly the chemistry strategy for that coupling may vary widely, and thus many combinations of the variables Y¹ and L¹ are possible. However, when the inhibitor is bound to the enzyme at its active site, the amino acid ester motif generally extends in a direction away from the enzyme, and thus minimises or avoids interference with the binding mode of the inhibitor. Hence the precise combination of variable making up the linking chemistry between the amino acid ester motif and the rest of the molecule will often be irrelevant to the primary binding mode of the compound as a whole.

With the foregoing general observations in mind, taking the variables making up the radical -L¹-Y¹— in turn:
Y¹ may be, for example, —NR₃—, —S—, —O—, —C(═O)NR₃—, —NR₃C(═O)—, or —C(═O)O—, wherein R₃ is hydrogen or optionally substituted C₁-C₆ alkyl such as —CH₂CH₂OH;
In the radical L¹, examples of Alk¹ and Alk² radicals, when present, include —CH₂—, —CH₂CH₂— —CH₂CH₂CH₂—, —CH₂CH(OH)CH₂—, —CH₂CH₂CH₂CH₂—, —CH═CH—, —CH═CHCH₂—, —CH₂CH═CH—, CH₂CH═CHCH₂—, —C≡C—, —C≡CCH₂—, —CH₂C≡C—, and CH₂C≡CCH₂. Additional examples of Alk¹ and Alk² include, in either orientation, —CH₂W—, —CH₂CH₂W—, —CH₂CH₂WCH₂—, —CH₂CH₂WCH(CH₃)—, —CH₂WCH₂CH₂—, —CH₂WCH₂CH₂WCH₂—, and —WCH₂CH₂— where W is —O—, —S—, —NH—, —N(CH₃)—, or —CH₂CH₂N(CH₂CH₂OH)CH₂—. Further examples of Alk¹ and Alk² include divalent cyclopropyl, cyclopentyl and cyclohexyl radicals.

Alk¹ and Alk² when present may also be branched chain alkyl such as —CH(CH₃)—, —C(CH₃)₂—, or in either orientation —CH₂CH(CH₃)—, —CH₂C(CH₃)₂—.

In L¹, when n is 0, the radical is a hydrocarbon chain (optionally substituted for example by hydroxyl) and perhaps having an ether, thioether or amino linkage). Presently it is preferred that there be no optional substituents in L¹. When both m and p are 0, L¹ is a divalent mono- or bicyclic carbocyclic or heterocyclic radical with 5-13 ring atoms (optionally substituted). When n is 1 and at least one of m and p is 1, L¹ is a divalent radical including a hydrocarbon chain or chains and a mono- or bicyclic carbocyclic or heterocyclic radical with 5-13 ring atoms (optionally substituted). When present, Q may be, for example, a divalent phenylene, pyridinylene, pyrimidinylene, pyrazinylene, piperidinylene, piperazinylene, pyrrolidenylene, pyrrolene, cyclopropylene, cyclobutylene, cyclopentylene, cyclohexylene or 3-aza-bicyclo[3.1.0]hexylene, radical, but 1,4-phenylene, 1,4-piperidinylene, or 1,4-piperazinyl are presently preferred.

Specific examples of the radical -L¹-Y¹— include those present in the compounds of the Examples herein.

A particular subclass of compounds of the invention consists of those of formula (IA)

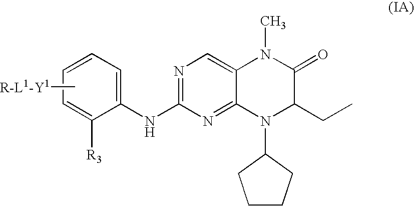

wherein R₃ is methoxy, fluoro or chloro, and the remaining variables are as defined and discussed above.

As mentioned above, the compounds with which the invention is concerned are inhibitors of PLK1 kinase activity and are therefore of use for treatment of cell proliferative diseases such as cancer.

It will be understood that the specific dose level for any particular patient will depend upon a variety of factors including the activity of the specific compound employed, the age, body weight, general health, sex, diet, time of administration, route of administration, rate of excretion, drug combination and the severity of the particular disease undergoing treatment. Optimum dose levels and frequency of dosing will be determined by clinical trial.

The compounds with which the invention is concerned may be prepared for administration by any route consistent with their pharmacokinetic properties. The orally administrable compositions may be in the form of tablets, capsules, powders, granules, lozenges, liquid or gel preparations, such as oral, topical, or sterile parenteral solutions or suspensions. Tablets and capsules for oral administration may be in unit dose presentation form, and may contain conventional excipients such as binding agents, for example syrup, acacia, gelatin, sorbitol, tragacanth, or polyvinyl-pyrrolidone; fillers for example lactose, sugar, maize-starch, calcium phosphate, sorbitol or glycine; tabletting lubricant, for example magnesium stearate, talc, polyethylene glycol or silica; disintegrants for example potato starch, or acceptable wetting agents such as sodium lauryl sulphate. The tablets may be coated according to methods well known in normal pharmaceutical practice. Oral liquid preparations may be in the form of, for example, aqueous or oily suspensions, solutions, emulsions, syrups or elixirs, or may be presented as a dry product for reconstitution with water or other suitable vehicle before use. Such liquid preparations may contain conventional additives such as suspending agents, for example sorbitol, syrup, methyl cellulose, glucose syrup, gelatin hydrogenated edible fats; emulsifying agents, for example lecithin, sorbitan monooleate, or acacia; non-aqueous vehicles (which may include edible oils), for example almond oil, fractionated coconut oil, oily esters such as glycerine, propylene glycol, or ethyl alcohol; preservatives, for example methyl or propyl p-hydroxybenzoate or sorbic acid, and if desired conventional flavouring or colouring agents.

For topical application to the skin, the drug may be made up into a cream, lotion or ointment. Cream or ointment formulations which may be used for the drug are conventional formulations well known in the art, for example as described in standard textbooks of pharmaceutics such as the British Pharmacopoeia.

For topical application by inhalation, the drug may be formulated for aerosol delivery for example, by pressure-driven jet atomizers or ultrasonic atomizers, or preferably by propellant-driven metered aerosols or propellant-free administration of micronized powders, for example, inhalation capsules or other “dry powder” delivery systems. Excipients, such as, for example, propellants (e.g. Frigen in the case of metered aerosols), surface-active substances, emulsifiers, stabilizers, preservatives, flavourings, and fillers (e.g. lactose in the case of powder inhalers) may be present in such inhaled formulations. For the purposes of inhalation, a large number of apparata are available with which aerosols of optimum particle size can be generated and administered, using an inhalation technique which is appropriate for the patient. In addition to the use of adaptors (spacers, expanders) and pear-shaped containers (e.g. Nebulator®, Volumatic®), and automatic devices emitting a puffer spray (Autohaler®), for metered aerosols, in particular in the case of powder inhalers, a number of technical solutions are available (e.g. Diskhaler®, Rotadisk®, Turbohaler® or the inhalers for example as described in European Patent Application EP 0 505 321).

For topical application to the eye, the drug may be made up into a solution or suspension in a suitable sterile aqueous or non aqueous vehicle. Additives, for instance buffers such as sodium metabisulphite or disodium edeate; preservatives including bactericidal and fungicidal agents such as phenyl mercuric acetate or nitrate, benzalkonium chloride or chlorhexidine, and thickening agents such as hypromellose may also be included.

The active ingredient may also be administered parenterally in a sterile medium. Depending on the vehicle and concentration used, the drug can either be suspended or dissolved in the vehicle. Advantageously, adjuvants such as a local anaesthetic, preservative and buffering agents can be dissolved in the vehicle.

The compounds of the invention may be used in conjunction with a number of known pharmaceutically active substances. For example, the compounds of the invention may be used with cytotoxics, HDAC inhibitors, kinase inhibitors, aminopeptidase inhibitors, protease inhibitors, bcl-2 antagonists, inhibitors of mTor and monoclonal antibodies (for example those directed at growth factor receptors). Preferred cytotoxics include, for example, taxanes, platins, anti-metabolites such as 5-fluoracil, topoisomerase inhibitors and the like. The medicaments of the invention comprising amino acid derivatives of formula (I), tautomers thereof or pharmaceutically acceptable salts, N-oxides, hydrates or solvates thereof therefore typically further comprise a cytotoxic, an HDAC inhibitor, a kinase inhibitor, an aminopeptidase inhibitor and/or a monoclonal antibody.

Further, the present invention provides a pharmaceutical composition comprising:
(a) a compound (I), or a pharmaceutically acceptable salt, N-oxide, hydrate or solvate thereof;
(b) a cytotoxic agent, an HDAC inhibitor, a kinase inhibitor, an aminopeptidase inhibitor, a protease inhibitor, a bcl-2 antagonist, an inhibitor of mTor and/or a monoclonal antibody; and
(c) a pharmaceutically acceptable carrier or diluent.

Also provided is a product comprising:
(a) a compound (I), or a pharmaceutically acceptable salt, N-oxide, hydrate or solvate thereof; and
(b) a cytotoxic agent, an HDAC inhibitor, a kinase inhibitor, an aminopeptidase inhibitor, a protease inhibitor, a bcl-2 antagonist, an inhibitor of mTor and/or a monoclonal antibody,
   for the separate, simultaneous or sequential use in the treatment of the human or animal body.

### Synthesis

There are multiple synthetic strategies for the synthesis of the compounds (I) with which the present invention is concerned, but all rely on known chemistry, known to the synthetic organic chemist. Thus, compounds according to formula (I) can be synthesised according to procedures described in the standard literature and are well-known to those skilled in the art. Typical literature sources are “Advanced organic chemistry”, 4^{th} Edition (Wiley), J March; “Comprehensive Organic Transformation”, 2^{nd} Edition (Wiley), R. C. Larock, “Handbook of Heterocyclic Chemistry”, 2^{nd} Edition (Pergamon), A. R. Katritzky; review articles such as found in “Synthesis”, “Acc. Chem. Res.”, “Chem. Red”, or primary literature sources identified by standard literature searches online or from secondary sources such as “Chemical Abstracts” or “Beilstein”.

The compounds of the invention may be prepared by a number of processes some of which are described specifically in the Examples below. In the reactions described below, it may be necessary to protect reactive functional groups, for example hydroxyl, amino and carboxy groups, where these are desired in the final product, to avoid their unwanted participation in the reactions [see for example, “Protecting Groups in Organic Synthesis”, 3^{rd} Edition, (Wiley), T. W. Greene]. Conventional protecting groups may be used in conjunction with standard practice. In some instances deprotection may be the final step in the synthesis of a compound of general formula (I), and the processes according to the invention described herein after are understood to extend to such removal of protecting groups.

### Abbreviations

AcOH=acetic acid
Boc or boc=tert-butoxycarbonyl

### BOC₂O=Di-tert-butyldicarbonate

Cbz=benzyloxycarbonyl
DBU=1,8-diazabicyclo[5.4.0]undec-7-ene
DCE=dichloroethane
DCM=dichloromethane
DIPEA=diisopropylethylamine
DMAP=dimethylaminopyridine
DMF=dimethylformamide
DMSO=dimethyl sulfoxide
EDC=1-ethyl-3-(3-dimethylaminopropyl)carbodiimide
EtOAc=ethyl acetate
EtOH=ethanol
Et₂O=diethyl ether
Et₃N=triethylamine
H₂SO₄=sulphuric acid
HCl=hydrochloric acid

### HOBt=N-hydroxybenzotriazole

K₂CO₃=potassium carbonate
LiOH=lithium hydroxide
MeOH=methanol
MgSO₄=magnesium sulphate
Na₂CO₃=sodium carbonate
NaH=sodium hydride
NaHCO₃=sodium hydrogen carbonate
NaI=sodium iodide
NaOH=sodium hydroxide
NBS=N-bromo succinimide
NBu₄Br=tetrabutylammonium bromide
NMM=N-methyl morpholine
Pd(dppf)Cl₂=dichloro-(1,2-bis-(diphenylphosphino)ethane)-palladium(II)
Pd/C=palladium on carbon
PPh₃=triphenyl phosphine

### PyBrOP=Bromo-tris-pyrrolidinophosphoniumhexafluorophosphate

STAB=sodium triacetoxyborohydride
TBTU=O-benzotriazol-1-yl-N,N,N′,N′-tetramethyluronium tetrafluoroborate
TFA=trifluoroacetic acid
THF=tetrahydrofuran
aq=aqueous
g=gram(s)
LCMS=high performance liquid chromatography/mass spectrometry
mg=milligram(s)
min=minutes
mL=milliliter(s)
μL=microlitre(s)
mol=mole(s)
mmol=millimole(s)
NMR=nuclear magnetic resonance
RT or rt=room temperature
sat=saturated

Commercially available reagents and solvents (HPLC grade) were used without further purification. Solvents were removed using a Buchi rotary evaporator. Microwave irradiation was carried out using a Biotage Initiator™ Eight microwave synthesiser. Purification of compounds by flash chromatography column was performed using silica gel, particle size 40-63 μm (230-400 mesh) obtained from Fluorochem. Purification of compounds by preparative HPLC was performed on Gilson systems using reverse phase Axia™ prep Luna C18 columns (10 μm, 100×21.2 mm), gradient 0-100% B (A=water/0.05% TFA, B=acetonitrile/0.05% TFA) over 10 min, flow=25 mL/min, UV detection at 254 nm.

¹H NMR spectra were recorded on a Bruker 300 MHz AV spectrometer in deuterated solvents. Chemical shifts (δ) are in parts per million. Thin-layer chromatography (TLC) analysis was performed with Kieselgel 60 F₂₅₄ (Merck) plates and visualized using UV light.

Analytical HPLC/MS was performed on an Agilent HP1100 LC system using reverse phase Luna C18 columns (3 μm, 50×4.6 mm), gradient 5-95% B (A=water/0.1% Formic acid, B=acetonitrile/0.1% Formic acid) over 2.25 min, flow=2.25 mL/min. UV spectra were recorded at 220 and 254 nm using a G1315B DAD detector. Mass spectra were obtained over the range m/z 150 to 800 on a LC/MSD SL G1956B detector. Data were integrated and reported using ChemStation and ChemStation Data Browser softwares.

### INTERMEDIATES

The intermediates for the preparation of the examples described herein are shown below (FIG. 1):

FIG. 1

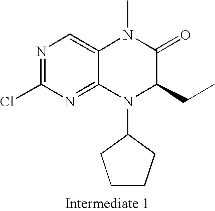

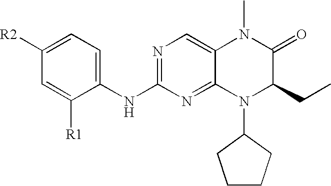

| R1 | R2 | Intermediate |
|---|---|---|
| —H | —OH | 2A |
| —OMe | —OH | 2B |
| —OMe | —CO₂H | 2C |
| —Me | —CO₂H | 2D |
| —F | —CO₂H | 2E |
| —H | —I | 2F |

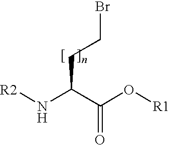

| R1 | R2 | n | Intermediate |
|---|---|---|---|
| -cyclopentyl | Boc | 1 | 3A |
| -^{t}butyl | Cbz | 1 | 3B |
| -cyclopentyl | Boc | 2 | 3C |
| -^{t}butyl | Cbz | 2 | 3D |

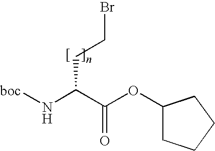

| n | Intermediate |
|---|---|
| 1 | 4A |
| 2 | 4B |

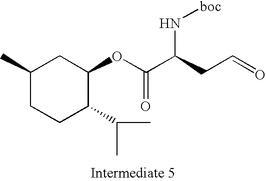

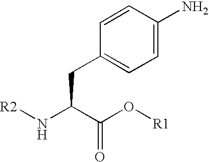

| R1 | R2 | Intermediate |
|---|---|---|
| -cyclopentyl | Boc | 6A |
| -^{t}butyl | Boc | 6B |

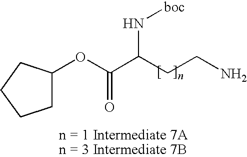

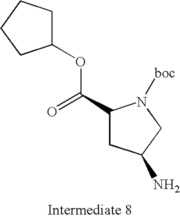

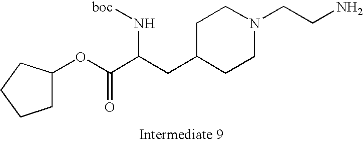

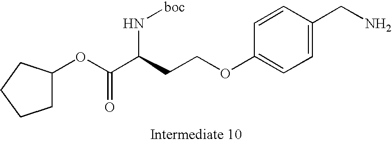

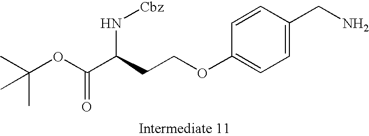

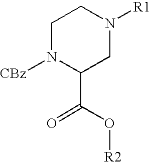

| R1 | R2 | Intermediate |
|---|---|---|
| —H | -cyclopentyl | 12A |
| —CH₂CH₂NH₂ | -cyclopentyl | 12B |
| —CH₂CH₂NH₂ | -^{t}butyl | 12C |

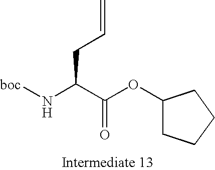

### Intermediate 1

### (7R)-2-Chloro-8-cyclopentyl-7-ethyl-5-methyl-7,8-dihydropteridin-6(5H)-one

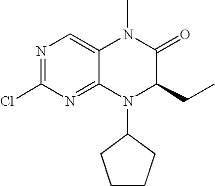

The title intermediate was prepared using methodology described in WO2004076454.

### Intermediates 2A-2F

### General Procedure

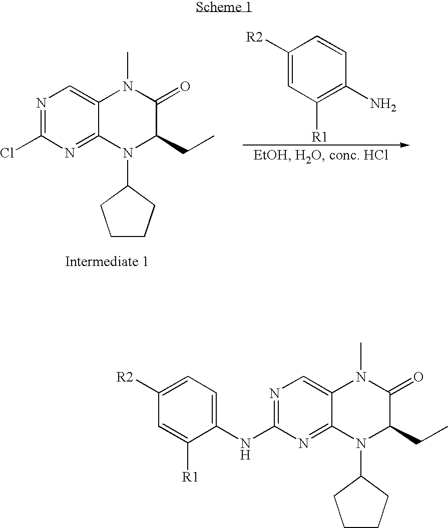

### Intermediate 2A

### (7R)-8-Cyclopentyl-7-ethyl-2-[(4-hydroxyphenyl)amino]-5-methyl-7,8-dihydropteridin-6(5H)-one

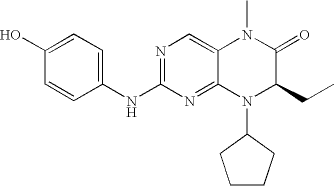

The title intermediate was prepared from Intermediate 1 according to the general procedure (Scheme 1).

To a solution of (7R)-2-chloro-8-cyclopentyl-7-ethyl-5-methyl-7,8-dihydropteridin-6(5H)-one [Intermediate 1] (200 mg, 0.68 mmol) in EtOH (2 mL), water (8 mL) and concentrated HCl (0.2 mL) was added 4-aminophenol (148 mg, 1.36 mmol). The reaction mixture was refluxed for 18 hours and concentrated under reduced pressure. The residue was partitioned between sat. NaHCO₃ (20 mL) and a mixture of MeOH/DCM (1:3, 20 mL). The aqueous layer was separated and extracted with MeOH/DCM (1:3, 20 mL). The combined organic layers were dried (MgSO₄) and concentrated under reduced pressure to leave a brown solid. Trituration with Et₂O afforded the titled intermediate as a grey solid (125 mg, 50% yield). ESMS: m/z 368 [M+H]⁺. ¹H NMR (DMSO-d₆, 300 MHz) 8.90 (1H, s), 8.64 (1H, s), 7.74 (1H, s), 7.43 (2H, d, J=8.9 Hz), 6.64 (2H, d, J=8.9 Hz), 4.39-4.29 (1H, m), 4.16 (1H, dd, J=3.6, 7.8 Hz), 3.22 (3H, s), 1.99-1.54 (10H, m), 0.77 (3H, t, J=7.4 Hz).

The intermediates in the table below were prepared by methods analogous to the method described above.

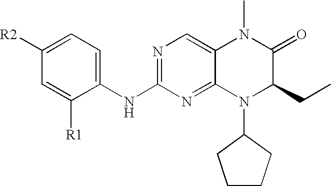

| Intermediate | R1 | R2 | Name | ESMS |
|---|---|---|---|---|
| 2B | —OMe | —OH | (7R)-8-Cyclopentyl-7-ethyl-2-[(4-hydroxy | m/z 398 |
| | | | 2-methoxyphenyl)amino]-5-methyl-7,8- | [M + H]⁺ |
| | | | dihydropteridin-6(5H)-one | |
| 2C | —OMe | —CO₂H | 4-{[(7R)-8-Cyclopentyl-7-ethyl-5-methyl-6- | m/z 426 |
| | | | oxo-5,6,7,8-tetrahydropteridin-2-yl] | [M + H]⁺ |
| | | | amino}-3-methoxybenzoic acid | |
| 2D | —Me | —CO₂H | 4-{[(7R)-8-Cyclopentyl-7-ethyl-5-methyl-6- | m/z 410 |
| | | | oxo-5,6,7,8-tetrahydropteridin-2-yl] | [M + H]⁺ |
| | | | amino}-3-methylbenzoic acid | |
| 2E | —F | —CO₂H | 4-{[(7R)-8-Cyclopentyl-7-ethyl-5-methyl-6- | m/z 414 |
| | | | oxo-5,6,7,8-tetrahydropteridin-2-yl] | [M + H]⁺ |
| | | | amino}-3-fluorobenzoic acid | |
| 2F | —H | —I | (7R)-8-Cyclopentyl-7-ethyl-2-[(4-iodo- | m/z 478 |
| | | | phenyl)amino]-5-methyl-7,8- | [M + H]⁺ |
| | | | dihydropteridin-6(5H)-one | |

### Intermediate 3A

### Cyclopentyl (2S)-4-bromo-2-[(tert-butoxycarbonyl)amino]butanoate

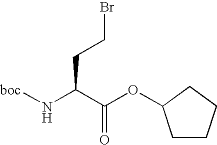

The title intermediate was prepared according to the procedure outlined below (Scheme 2).

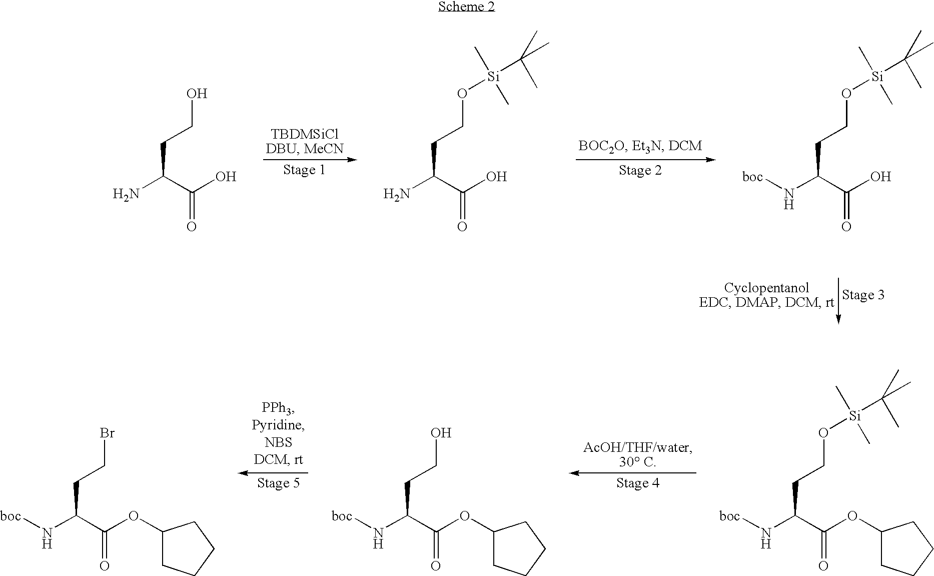

### Stage 1—O-[tert-butyl(dimethyl)silyl]-L-homoserine

To a suspension of L-homoserine (1.00 g, 8.40 mmol) in acetonitrile (10 mL) at 0° C. was added DBU (1.32 mL, 8.80 mmol,). tert-Butyl-dimethyl silyl chloride (1.33 g, 8.80 mmol) was then added portionwise over 5 minutes and the reaction mixture allowed to warm to RT and stirred for 16 hours. The white solid was filtered and washed with acetonitrile to give the product (1.80 g, 92% yield). ESMS: m/z 234 [M+H]⁺.

### Stage 2—N-(tert-butoxycarbonyl)-O-[tert-butyl(dimethyl)silyl]-L-homoserine

To a suspension of O-[tert-butyl(dimethyl)silyl]-L-homoserine (1.80 g, 7.70 mmol) in DCM (100 mL) at 0° C. was added Et₃N (2.15 mL, 15.4 mmol) and BOC₂O (1.77 g, 8.10 mmol). The reaction mixture was stirred at RT for 16 hours. The DCM was removed under reduced pressure and the residue was re-dissolved in EtOAc (20 ml) and brine (10 ml). The EtOAc layer was dried (MgSO₄) and concentrated under reduced pressure to give the crude product which was taken forward without further purification (2.53 g, 99% yield). ESMS: m/z 356 [M+H]⁺.

### Stage 3—Cyclopentyl N-(tert-butoxycarbonyl)-O-[tert-butyl(dimethyl)silyl]-L-homoserinate

To a solution of N-(tert-butoxycarbonyl)-O-[tert-butyl(dimethyl)silyl]-L-homoserine (2.53 g, 7.6 mmol) in DCM (50 mL) at 0° C. was added cyclopentanol (1.39 mL, 15.3 mmol), EDC (1.61 g, 8.40 mmol) and DMAP (93 mg, 0.76 mmol). The reaction mixture was stirred for 16 hours at RT before concentration under reduced pressure. The crude residue was dissolved in EtOAc (100 mL) and washed with 1M HCl (30 ml), 1M Na₂CO₃ (30 ml) and brine (20 ml). The organic layer was dried (MgSO₄) and concentrated under reduced pressure. The residue was purified by column chromatography (25% EtOAc/heptane) to afford the product (2.24 g, 73% yield). ESMS: m/z 402 [M+H]⁺.

### Stage 4—Cyclopentyl N-(tert-butoxycarbonyl)-L-homoserinate

A solution of cyclopentyl N-(tert-butoxycarbonyl)-O-[tert-butyl(dimethyl)silyl]-L-homoserinate (1.57 g, 3.90 mmol) in acetic acid:THF:water (3:1:1, 100 mL) was stirred at 30° C. for 16 hours. EtOAc (200 mL) was added and washed with 1M Na₂CO₃ (10 ml), 1M HCl (10 ml) and brine (10 ml). The EtOAc layer was dried (MgSO₄) and concentrated under reduced pressure to afford the product as a clear oil which solidified on standing (1.00 g, 95% yield). ESMS: m/z 310 [M+Na]⁺.

### Stage 5—Cyclopentyl (2S)-4-bromo-2-[(tert-butoxycarbonyl)amino]butanoate

To a suspension of NBS (1.86 g, 10.4 mmol) in DCM (16 mL) was added a solution of triphenyl phosphine (2.56 g, 9.70 mmol) in DCM (7 mL). The solution was stirred for 5 minutes after addition. Pyridine (0.34 mL, 4.20 mmol) was added followed by a solution of cyclopentyl N-(tert-butoxycarbonyl)-L-homoserinate (1.00 g, 3.5 mmol) in DCM (9 mL). The solution was stirred at RT for 18 hours, concentrated under reduced pressure and the residual solvent azeotroped with toluene (3×16 mL). The residue was triturated with Et₂O (10 mL) and EtOAc:heptane (1:9, 2×10 mL). The combined organic solutions were concentrated onto silica and purified by column chromatography (10%-25% EtOAc/heptane) to afford the title intermediate (1.02 g, 84% yield). ESMS: m/z 351 [M+H]⁺. ¹H NMR (300 MHz, CDCl₃) 5.30-5.05 (2H, m), 4.45-4.30 (1H, m), 3.45 (2H, t, J=7.3 Hz), 2.50-2.30 (1H, m), 2.25-2.10 (1H, m), 1.95-1.60 (8H, br m) and 1.47 (9H, s).

### Intermediate 3B

### tert-butyl (2S)-2-{[(benzyloxy)carbonyl]amino}-4-bromobutanoate

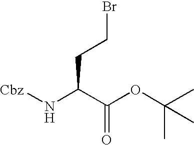

The title intermediate was prepared according to the procedure outlined below (Scheme 3).

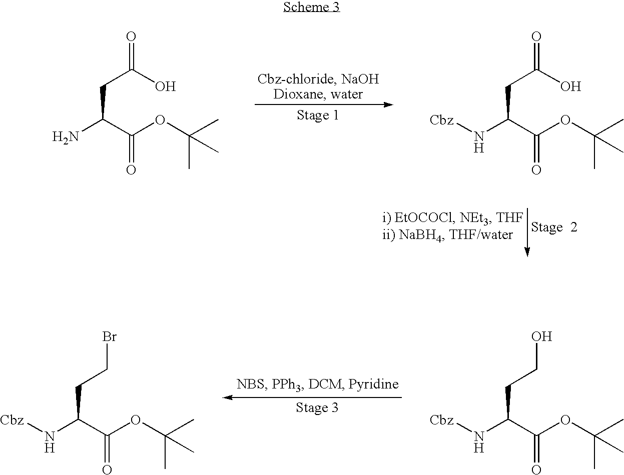

### Stage 1—(3S)-3-{[(Benzyloxy)carbonyl]amino}-4-tert-butoxy-4-oxobutanoic acid

To a solution of (3S)-3-amino-4-tert-butoxy-4-oxobutanoic acid (900 mg, 4.75 mmol) and sodium hydroxide (280 mg, 7.13 mmol) in 25% water/dioxane (50 mL) at 0° C. was added benzyl chloroformate (2 g, 4.13 mmol) in dioxane (10 mL). The mixture was stirred at 0° C. for 1 hour and then at RT overnight. Water (10 mL) was added and the mixture was extracted with EtOAc (2×20 mL). The organic phase was back extracted with a saturated aqueous solution of NaHCO₃ (2×10 mL). The combined aqueous layers were acidified to pH 1 with 1M HCl, and extracted with EtOAc (3×10 mL). The combined organic layers were dried (MgSO₄) and concentrated under reduced pressure. The residue was purified by column chromatography (35% EtOAc/heptane) to give the product as a colourless oil (0.76 g, 50% yield). ESMS: m/z 346 [M+23]⁺

### Stage 2—tert-Butyl N-[(benzyloxy)carbonyl]-L-homoserinate

To a solution of (3S)-3-{[(benzyloxy)carbonyl]amino}-4-tert-butoxy-4-oxobutanoic acid (600 mg, 1.87 mmol) in anhydrous THF (20 mL) at −20° C. was slowly added Et₃N (32 μL, 2.24 mmol) and ethyl chloroformate (21 μL, 2.24 mmol). The mixture was stirred at −20° C. for 2 hours. The solid formed was filtered off and washed with THF (2×10 mL). The filtrate was added dropwise to a solution of sodium borohydride (0.2 g, 5.61 mmol) at 0° C. over 10 minutes and then allowed to warm to RT. The mixture was stirred for an additional 4 hours. The solvent was removed under reduced pressure and the residue was diluted with water (10 mL), acidified to pH 5 with 1M HCl and extracted with EtOAc (2×20 ml). The combined organic fractions were washed with 10% aqueous NaOH (10 mL), water (10 mL) and brine (10 mL). The organic layer was dried (MgSO₄) and concentrated under reduced pressure to give the product as a clear oil (0.3 g, 51% yield). ESMS: m/z 332 [M+23]⁺.

### Stage 3—tert-butyl (2S)-2-{[(benzyloxy)carbonyl]amino}-4-bromobutanoate

To a solution of NBS (520 mg, 2.91 mmol) in DCM (10 mL) was slowly added a solution of triphenylphosphine (0.71 g, 2.72 mmol) in DCM (10 mL). The mixture was stirred at RT for 5 minutes before pyridine (94 μL, 1.16 mmol) and a solution of tert-butyl N-[(benzyloxy)carbonyl]-L-homoserinate (0.30 g, 0.97 mmol) in DCM (20 mL) were added dropwise. The mixture was stirred at RT for another 18 hours. The solvent was removed under reduced pressure, the residue was azeotroped with toluene (2×15 mL) and triturated with Et₂O (2×25 mL) and 10% EtOAc in heptanes. The filtrate from the triturations were combined and concentrated under reduced pressure. The crude product was purified by column chromatography (15% EtOAc/heptanes) to give the title intermediate as a clear oil (0.16 g, 44% yield). ESMS: m/z 395 [M+23]⁺. ¹H NMR (300 MHz, CDCl₃), δ ppm 7.39-7.30 (5H, m), 5.40 (1H, d, J=6.8 Hz), 5.12 (2H, s), 4.38 (1H, q, J=7.7 Hz), 3.47-3.38 (2H, m), 5.49-2.33 (1H, m), 2.28-2.13 (1H, m) and 1.48 (9H, s).

### Intermediate 3C

### Cyclopentyl 5-bromo-N-(tert-butoxycarbonyl)-L-norvalinate

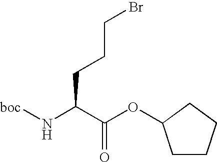

The title intermediate was prepared according to the procedure outlined below (Scheme 4).

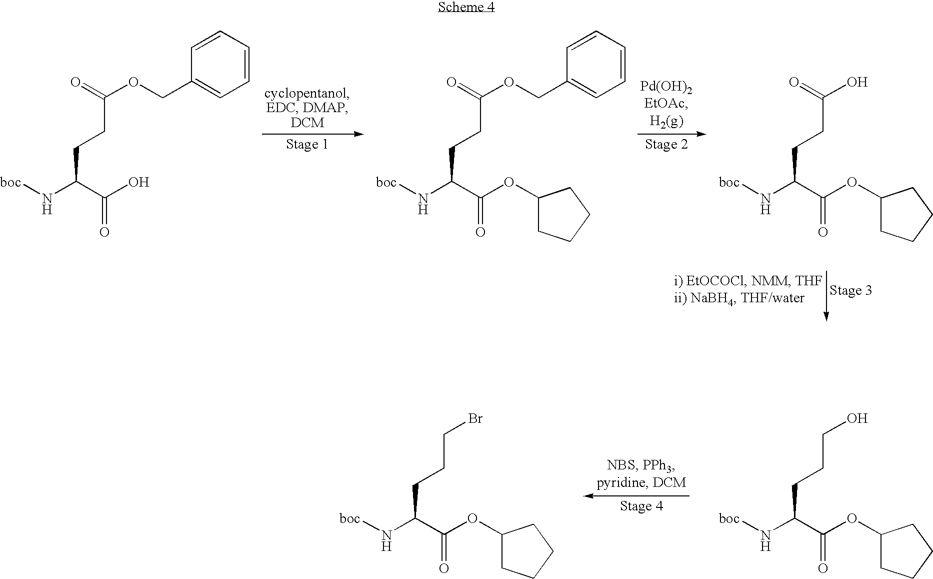

### Stage 1—5-Benzyl 1-cyclopentyl N-(tert-butoxycarbonyl)-L-glutamate

To a solution of (2S)-5-(benzyloxy)-2-[(tert-butoxycarbonyl)amino]-5-oxopentanoic acid (15 g, 44.5 mmol) in DCM (220 mL) at 0° C. was added cyclopentanol (4.8 mL, 53.3 mmol), EDC (9.4 g, 48.9 mmol) and DMAP (543 mg, 4.4 mmol). The reaction mixture was allowed to warm to RT and stirred for a further 12 hours. The reaction mixture was diluted with DCM (200 mL) and washed with 1M HCl (50 mL), 1M Na₂CO₃ (30 mL) and brine (50 mL). The organic layer was dried (MgSO₄) and concentrated under reduced pressure. The residue was purified by column chromatography (25% EtOAc/heptane) to give the product as a white solid (12.4 g, 69% yield). ESMS: m/z 406 [M+H]⁺.

### Stage 2—1-Cyclopentyl N-(tert-butoxycarbonyl)-L-glutamatic acid

5-Benzyl 1-cyclopentyl N-(tert-butoxycarbonyl)-L-glutamate (12.4 g, 30.5 mmol) was dissolved in EtOAc (200 mL) and purged with nitrogen before addition of Pd(OH)₂ on carbon catalyst (1.3 g, 20% w/w). The reaction flask was then purged with hydrogen gas for a period of 5 minutes before leaving under a balloon of hydrogen for 5 hours. The catalyst was removed by filtration through Celite®, washing thoroughly with EtOAc (50 mL). The solvent was removed under reduced pressure to give the product as a clear oil (7.73 g, 85% yield). ESMS: m/z 316 [M+H]⁺.

### Stage 3—Cyclopentyl N-(tert-butoxycarbonyl)-5-hydroxy-L-norvalinate

To a stirred solution of 1-cyclopentyl N-(tert-butoxycarbonyl)-L-glutamatic acid (6.73 g, 21.4 mmol) in THF (150 mL) at −20° C. was added NMM (3.05 mL, 27.8 mmol) and ethyl chloroformate (2.45 mL, 25.6 mmol). The reaction mixture was stirred at −20° C. for 2 hours. The solid was removed by filtration was added dropwise over 20 minutes to a solution of sodium borohydride (2.43 g, 64.1 mmol) in THF (20 mL) and water (5 mL) at 0° C. The reaction mixture was allowed to warm to RT and left for a further 4 hours. The mixture was acidified to pH 5 with 1M HCl and the THF removed under reduced pressure. The aqueous solution was extracted with EtOAc (3×100 mL), dried (MgSO₄), and concentrated under reduced pressure. The residue was purified by column chromatography (0-5% MeOH/DCM) to give the product as a clear oil (5.0 g, 78% yield). ESMS: m/z 302 [M+H]⁺.

### Stage 4—Cyclopentyl 5-bromo-N-(tert-butoxycarbonyl)-L-norvalinate

To a suspension of NBS (3.54 g, 19.9 mmol) in DCM (30 mL) was added a solution of triphenylphosphine (4.87 g, 18.8 mmol) in DCM (15 mL). The solution was stirred for a further 5 minutes before addition of pyridine (64 μL, 7.96 mmol) and a solution of cyclopentyl N-(tert-butoxycarbonyl)-5-hydroxy-L-norvalinate (2.0 g, 6.64 mmol) in DCM (20 mL). The solution was stirred for 18 hours, concentrated under reduced pressure and the residual solvent azeotroped with toluene (3×30 mL). The residue was triturated with Et₂O (30 mL) and 10% EtOAc/heptane (2×30 mL). The combined Et₂O and EtOAc/heptane solutions were concentrated onto silica and purified by column chromatography (10%-25% EtOAc/heptane) to give the title intermediate as a clear oil (1.34 g, 55% yield). ESMS: m/z 365 [M+H]⁺. ¹H NMR (300 MHz, CDCl3), δ: 5.25 (1H, m), 5.05 (1H, bd), 3.45 (2H, m), 2.00-1.55 (12H, bm) and 1.45 (9H, s).

### Intermediate 3D

### tert-butyl N-[(benzyloxy)carbonyl]-5-bromo-L-norvalinate

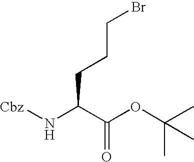

The title intermediate was prepared according to the procedure outlined for intermediate 3B [Scheme 3] starting with (4S)-4-amino-5-tert-butoxy-5-oxopentanoic acid. ESMS: m/z 409 [M+Na]⁺.

### Intermediate 4A

### Cyclopentyl (2R)-4-bromo-2-[(tert-butoxycarbonyl)amino]butanoate

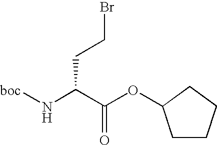

The title intermediate was prepared according to the procedure outlined for intermediate 3A [Scheme 2] starting with D-homoserine. ESMS: m/z 351 [M+H]⁺.

### Intermediate 4B

### Cyclopentyl 5-bromo-N-(tert-butoxycarbonyl)-D-norvalinate

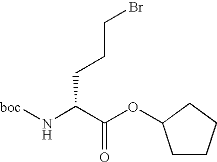

The title intermediate was prepared according to the procedure outlined for intermediate 3C [Scheme 4] starting with of (2R)-5-(benzyloxy)-2-[(tert-butoxycarbonyl)amino]-5-oxopentanoic acid. ESMS: m/z 365 [M+H]⁺.

### Intermediate 5

### (1R,2S,5R)-2-isopropyl-5-methylcyclohexyl (2S)-2-[(tert-butoxycarbonyl)amino]-4-oxobutanoate

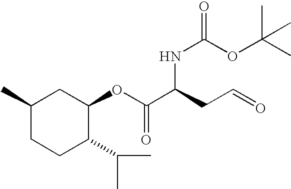

The title intermediate was prepared according to the procedure outlined below (Scheme 5).

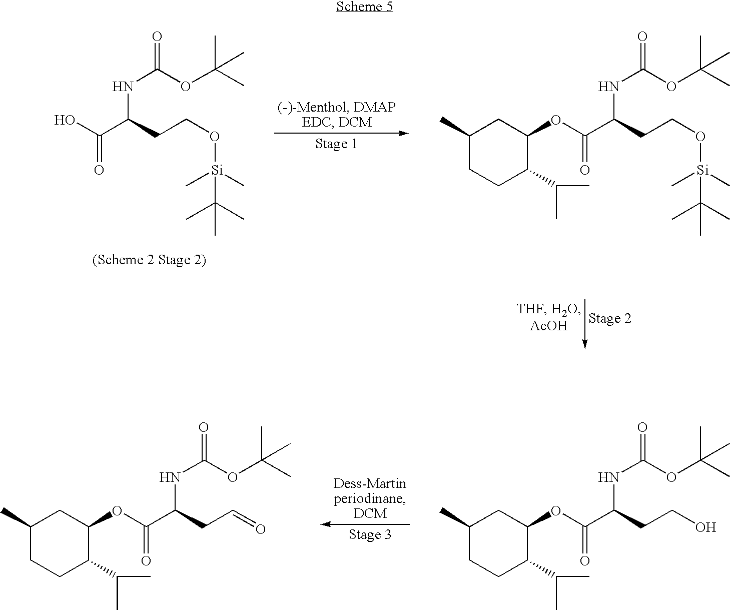

### Stage 1—(1R,2S,5R)-2-isopropyl-5-methylcyclohexyl N-(tert-butoxycarbonyl)-O-[tert-butyl(dimethyl)silyl]-homoserinate

To a suspension of N-(tert-butoxycarbonyl)-O-[tert-butyl(dimethyl)silyl]-L-homoserine [Scheme 2 Stage 2] (6.22 g, 19 mmol) in DCM (120 mL) at 0° C. was added (−)-menthol (5.85 g, 37.0 mmol), DMAP (228 mg, 1.87 mmol) and EDC (3.93 g, 20.3 mmol). The solution was allowed to warm to RT and stirred for a further 18 hours. The reaction mixture was concentrated under reduced pressure. The residue was purified by column chromatography (20% EtOAc/heptane) to give the product as a clear oil (4.86 g, 55% yield). ESMS: m/z 394 [M+Na]⁺.

### Stage 2—(1R,2S,5R)-2-isopropyl-5-methylcyclohexyl N-(tert-butoxycarbonyl)-L-homoserinate

A suspension of (1R,2S,5R)-2-isopropyl-5-methylcyclohexyl N-(tert-butoxycarbonyl)-O-[tert-butyl(dimethyl)silyl]-L-homoserinate (4.86 g, 14.0 mmol) in THF/water/acetic acid (60 mL:60 mL:180 mL) was heated at 30° C. for 20 hours. The reaction was diluted with EtOAc (60 mL) and washed with sat NaHCO₃ solution (20 mL), 1M HCl (30 mL) and brine (30 mL). The organic layer was dried (MgSO₄) and concentrated under reduced pressure to afford the product (3.45 g 69% yield). ESMS: m/z 380 [M+Na]⁺.

### Stage 3—(1R,2S,5R)-2-isopropyl-5-methylcyclohexyl (2S)-2-[(tert butoxycarbonyl)amino]-4-oxobutanoate

To a suspension of (1R,2S,5R)-2-isopropyl-5-methylcyclohexyl N-(tert-butoxycarbonyl)-L-homoserinate (500 mg, 1.40 mmol) in DCM (20 mL) at 0° C. was added Dess-Martin periodinane (595 mg, 1.54 mmol). The reaction was allowed to warm to RT and stirred for 3 hours. To the solution was added 1:1 Na₂SO₃/NaHCO₃ saturated solution (30 mL) and the mixture stirred for 15 min. The organic layer was separated and the aqueous layer extracted with DCM (2×10 mL). The combined organic layers were washed with 1:1 Na₂SO₃/NaHCO₃ solution (15 mL), dried (MgSO₄) and concentrated under reduced pressure to give the title intermediate as a colourless oil (480 g 97% yield). ESMS: m/z 378 [M+Na]⁺. ¹H NMR (CDCl₃) δ: 7.90 (1H, m), 5.30 (1H, d J=4.7 Hz), 4.70-4.57 (2H, m), 4.45 (1H, br. s), 2.92 (2H, t, J=5.7 Hz), 1.91-1.68 (6H, m), 1.58 (9H, s), 1.05-0.85 (4H, m) and 0.66 (6H, d, J=7.0 Hz).

### Intermediate 6A

### Cyclopentyl 4-amino-N-(tert-butoxycarbonyl)-L-phenylalaninate

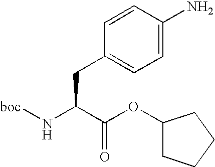

The title intermediate was prepared according to the procedure outlined below (Scheme 6).

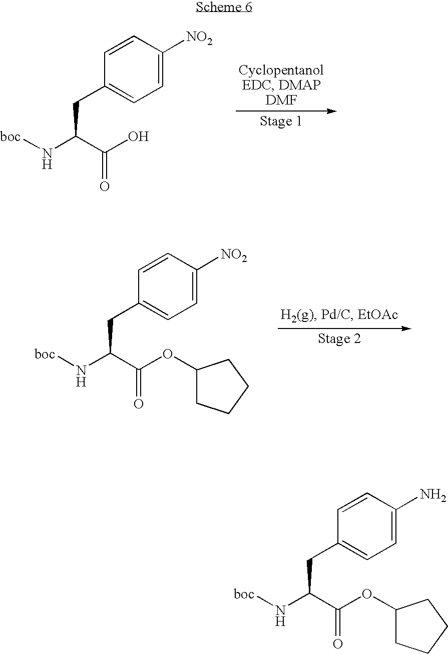

### Stage 1—Cyclopentyl N-(tert-butoxycarbonyl)-4-nitro-L-phenylalaninate

To a solution of N-(tert-butoxycarbonyl)-4-nitro-L-phenylalanine (1.00 g, 3.23 mmol) in DMF (10 mL) at 0° C. was added cyclopentanol (0.585 mL, 6.44 mmol), DMAP (39 mg, 0.32 mmol) and EDC (0.655 g, 3.39 mmol). The reaction mixture was allowed to warm to RT and stirred for a further 16 hours. The mixture was partitioned between water (200 mL) and EtOAc (200 mL). The organic layer was extracted with water (3×50 mL), dried (MgSO₄) and concentrated under reduced pressure. The residue was purified by column chromatography (33% heptane/EtOAc) to afford the product as a pale yellow oil (1.12 g, 95% yield). ESMS: m/z 365 [M+H]⁺.

### Stage 2—Cyclopentyl 4-amino-N-(tert-butoxycarbonyl)-L-phenylalaninate

To a solution of cyclopentyl N-(tert-butoxycarbonyl)-4-nitro-L-phenylalaninate (480 mg, 1.32 mmol) in EtOAc (10 mL) was added 10% Pd/C (48 mg, 10% w/w). The flask was evacuated and put under a hydrogen atmosphere for two hours. The reaction was evacuated and the mixture filtered through Celite®, washing with excess EtOAc (20 mL). The filtrate was concentrated under reduced pressure to afford the title intermediate as a pink oil (432 mg, 98% yield). ESMS: m/z 335 [M+H]⁺. ¹H NMR (300 MHz, CDCl₃) δ: 6.62 (2H, d, J=8.4 Hz), 5.15-5.25 (1H, m, CH), 4.95 (1H, d, J=4.2 Hz), 4.40-4.55 (1H, m), 6.94 (2H, d, J=8.1 Hz), 3.62 (2H, br s), 2.97 (2H, d, J=5.7 Hz), 1.50-1.96 (9H, m) and 1.44 (9H, s).

### Intermediate 6B

### tert-Butyl 4-amino-N-(tert-butoxycarbonyl)-L-phenylalaninate

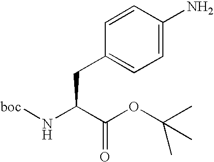

The title intermediate was prepared according to the procedure outlined below (Scheme 7).

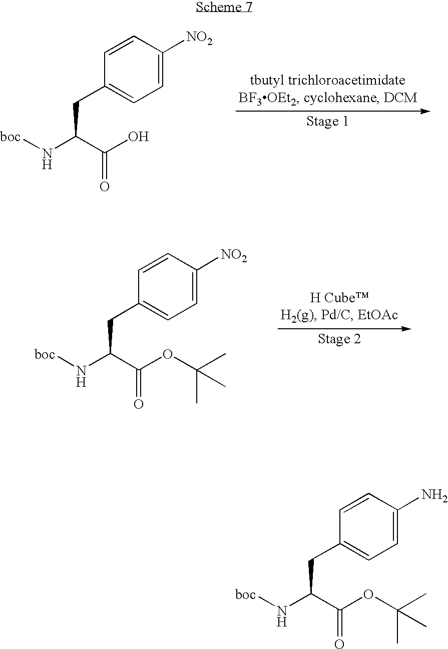

### Stage 1—tert-Butyl N-(tert-butoxycarbonyl)-4-nitro-L-phenylalaninate

To a solution of N-(tert-butoxycarbonyl)-4-nitro-L-phenylalanine (500 mg, 1.61 mmol) in 66% DCM/cyclohexane (30 mL) at 0° C. was added boron trifluoride diethyl etherate (10 μL) followed immediately by dropwise addition over 10 minutes of tert-butyl trichloroacetimidate (704 mg, 3.22 mmol) in cyclohexane (10 mL). The mixture was allowed to warm to RT and stirred for 30 minutes before quenching with NaHCO₃ powder (80 mg). The crude mixture was filtered through Celite® and the filtrate concentrated under reduced pressure to give a residue. The residue was purified by column chromatography (20% EtOAc/heptane) to give the product as a yellow solid (320 mg, 54% yield). ESMS: m/z 389 [M+Na]⁺.

### Stage 2—tert-Butyl 4-amino-N-(tert-butoxycarbonyl)-L-phenylalaninate

Stage 1 product (0.53 g, 1.40 mmol) was dissolved in MeOH (29 mL) to make a 0.05M solution. The solution was passed through an H-Cube™ continuous hydrogenator (Thales Nanotechnology, HC-2, SS). The reaction was performed using a 30 mm CatCart® (10% Pd/C) in full H₂ mode. A flow rate of 1 mL/min was maintained, with a temperature of 25° C. and H₂ pressure of 1 bar. The product was eluted into 2M NaOH (20 mL) and the MeOH removed under reduced pressure. The aqueous solution was extracted with EtOAc (2×20 mL). The combined organic layers were dried (MgSO₄) and concentrated under reduced pressure to afford the title intermediate as a yellow oil. (0.15 g, 31% yield). ESMS: m/z 359 [M+Na]⁺. ¹H NMR (300 MHz, MeOD) δ: 6.97 (2H, d, J=8.5 Hz), 6.68 (2H, d, J=8.3 Hz), 4.15 (1H, t, J=5.9 Hz), 2.85 (2H, dd, J=19.0, 7.2 Hz) and 1.42 (18H, s).

### Intermediate 7A

### Cyclopentyl (2S)-4-amino-2-[(tert-butoxycarbonyl)amino]butanoate

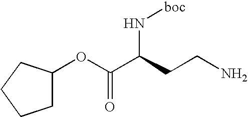

The title intermediate was prepared according to the procedure outlined below (Scheme 8).

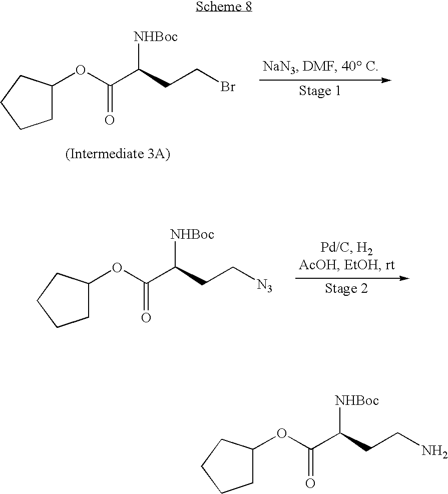

### Stage 1—Cyclopentyl (2S)-4-azido-2-[(tert-butoxycarbonyl)amino]butanoate

To a solution of cyclopentyl (2S)-4-bromo-2-[(tert-butoxycarbonyl)amino]butanoate [Intermediate 3A] (1.00 g, 2.90 mmol) in DMF (30 mL) was added sodium azide (0.93 g, 14.3 mmol). The reaction mixture was stirred at 40° C. for 32 hours and concentrated under reduced pressure. The residue was partitioned between Et₂O (100 mL) and sat. Na₂CO₃ (100 mL). The organic layer was separated, washed with sat. Na₂CO₃ (100 mL), and brine (100 mL), dried (MgSO₄), and concentrated under reduced pressure to give the product as a yellow oil (1.05 g). This product was used without further purification. ESMS: m/z 335 [M+Na]⁺

### Stage 2—Cyclopentyl (2S)-4-amino-2-[(tert-butoxycarbonyl)amino]butanoate

To a solution of crude cyclopentyl (2S)-4-azido-2-[(tert-butoxycarbonyl)amino]butanoate (1.05 g, 2.90 mmol) in ethanol (50 mL) was added acetic acid (0.16 mL, 2.90 mmol). The reaction mixture was flushed 3 times with nitrogen. Pd/C (50 mg, 10% w/w) was added. The mixture was flushed 3 times with nitrogen and finally stirred under an atmosphere of hydrogen at RT for 2 hours. The reaction mixture was filtered through a short pad of Celite® and the filtrate was concentrated under reduced pressure. The residue was partitioned between EtOAc (50 mL) and sat. Na₂CO₃ (50 mL). The organic layer was separated, washed with brine (50 mL), dried (MgSO₄), and concentrated under reduced pressure to leave a yellow oil. Purification by column chromatography (2% ammonia:5% MeOH in DCM) afforded the title intermediate as a colorless oil (638 mg, 78% yield over 2 steps). ESMS: m/z 287 [M+H]⁺. ¹H NMR (300 MHz, CDCl3) δ:5.55 (1H, d), 5.21 (1H, m), 4.35 (1H, m), 2.81 (2H, m), 1.89 (2H, m), 1.81-1.55 (8H, m) and 1.45 (9H, s).

### Intermediate 7B

### Cyclopentyl N²-(tert-butoxycarbonyl)lysinate

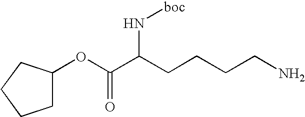

The title intermediate was prepared according to the procedure outlined below (Scheme 9).

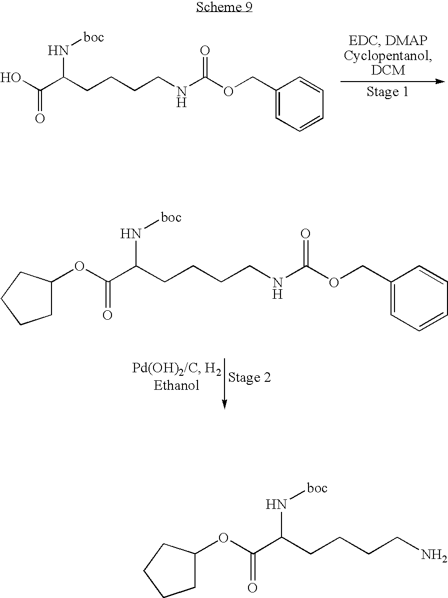

### Stage 1—Cyclopentyl N⁶-[(benzyloxy)carbonyl]-N²-(tert-butoxycarbonyl)lysinate

To a solution of N⁶-[(benzyloxy)carbonyl]-N²-(tert-butoxycarbonyl)lysine (1.00 g, 2.63 mmol) in anhydrous DCM (20 mL) at 0° C. was added DMAP (32 mg, 0.26 mmol), cyclopentanol (0.48 mL, 5.23 mmol) and EDC (552 mg, 2.89 mmol). The reaction was allowed to warm to room RT and stirred for a further 16 hours. The mixture was diluted with DCM (50 mL) and washed with brine (50 mL). The organic layer was dried (MgSO₄) and concentrated under reduced pressure to give crude product as an oil (1.18 g, 100% yield) which was used without further purification. ESMS: m/z 471 [M+Na]⁺.

### Stage 2—Cyclopentyl N²-(tert-butoxycarbonyl)lysinate

To a solution of cyclopentyl N⁶-[(benzyloxy)carbonyl]-N²-(tert-butoxycarbonyl)lysinate (1.18 g, 2.63 mmol) in ethanol (5 mL) was carefully added palladium hydroxide on carbon (235 mg, 20% w/w) under an atmosphere of nitrogen. The reaction mixture was evacuated and placed under an atmosphere of H₂. This was repeated a further two times and the reaction allowed to stir under and atmosphere of H₂ for 2 hours. The reaction mixture was filtered through Celite® and concentrated to give the title intermediate (250 mg). ESMS: m/z 315 [M+H]⁺. ¹H NMR (300 MHz, DMSO) δ: 6.70-6.77 (1H, m), 5.13-5.15 (1H, m), 4.08-4.09 (1H, m), 2.88-2.90 (2H, m), 1.82 (2H, m), 1.57-1.66 (10H, m) and 1.03-1.37 (11H, m).

### Intermediate 8

### 1-tert-Butyl 2-cyclopentyl (2S,4S)-4-aminopyrrolidine-1,2-dicarboxylate

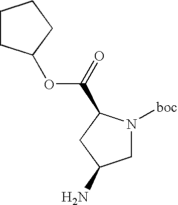

The title intermediate was prepared according to the procedure outlined below (Scheme 10).

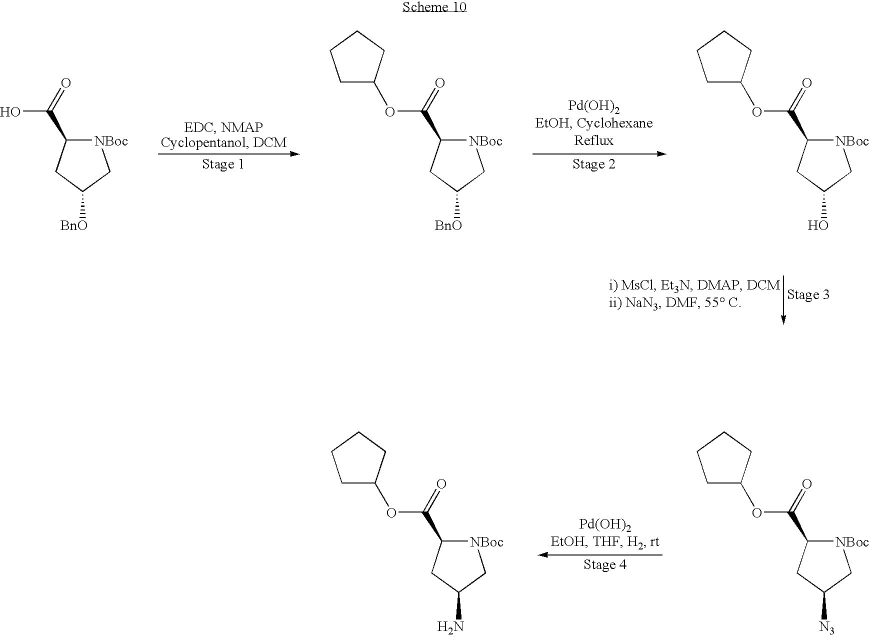

### Stage 1—1-tert-Butyl 2-cyclopentyl (2S,4R)-4-(benzyloxy)pyrrolidine-1,2-dicarboxylate

To a solution of (4R)-4-(benzyloxy)-1-(tert-butoxycarbonyl)-L-proline (5.06 g, 15.7 mmol) in DCM (50 mL) at 0° C. was added cyclopentanol (2.9 mL, 31.4 mmol), DMAP (192 mg, 1.60 mmol) and EDC (3.32 g, 17.3 mmol). The reaction mixture was allowed to warm to RT and stirred for a further 18 hours. The mixture was washed with sat, Na₂CO₃ (30 mL), 1M HCl (30 mL) and brine (30 mL). The organic layer was dried (MgSO₄) and concentrated under reduced pressure to leave a pale yellow oil. Purification by column chromatography (15% EtOAc/heptane) afforded the product as a colourless oil (5.21 g, 85% yield). ESMS: m/z 412 [M+Na]⁺ and 801 [2M+Na]⁺.

### Stage 2—1-tert-Butyl 2-cyclopentyl (2S,4R)-4-hydroxypyrrolidine-1,2-dicarboxylate

To a solution of 1-tert-butyl 2-cyclopentyl (2S,4R)-4-(benzyloxy)pyrrolidine-1,2-dicarboxylate (5.21 g, 13.4 mmol) in EtOH:cyclohexene (5:1, 120 mL) was carefully added palladium hydroxide on carbon (521 mg, 20% w/w) The reaction mixture was evacuated and flushed with nitrogen 3 times and refluxed for 21 hours. The reaction mixture was filtered through Celite® and the filtrate was concentrated under reduced pressure to leave a pale yellow oil. Purification by column chromatography (50% EtOAc/heptane) afforded the product as a pale pink oil (3.77 g, 100% yield). ESMS: m/z 621 [2M+Na]⁺.

### Stage 3—1-tert-Butyl 2-cyclopentyl (2S,4S)-4-(2I⁵-triaz-1-en-2-yn-1-yl)pyrrolidine-1,2-dicarboxylate

To a solution of 1-tert-butyl 2-cyclopentyl (2S,4R)-4-hydroxy-pyrrolidine-1,2-dicarboxylate (3.07 g, 10.3 mmol) in DCM (100 mL) at 0° C. was added Et₃N (2.90 mL, 20.5 mmol), DMAP (125 mg, 1.02 mmol) and methanesulfonyl chloride (0.87 mL, 11.3 mmol). The reaction mixture was allowed to warm to RT and stirred for 1 hour. The mixture was washed with water (50 mL) and brine (50 mL). The organic layer was dried (MgSO₄) and concentrated under reduced pressure. The residue was dissolved in DMF (100 mL) and sodium azide (100 mg, 15.5 mmol) was added. The reaction mixture was stirred at 60° C. for 3 days, allowed to cool to RT and partitioned between water (200 mL) and EtOAc (200 mL). The organic layer was separated, washed with brine (200 mL), dried (MgSO₄) and concentrated under reduced pressure to leave a pale yellow oil. Purification by column chromatography (30% EtOAc/heptane) afforded the title compound as a colourless oil (3.26 g, 98% yield). ESMS: m/z 671 [2M+Na]⁺

### Stage 4—1-tert-Butyl 2-cyclopentyl (2S,4S)-4-aminopyrrolidine-1,2-dicarboxylate

To a solution of 1-tert-butyl 2-cyclopentyl (2S,4S)-4-(2I⁵-triaz-1-en-2-yn-1-yl)pyrrolidine-1,2-dicarboxylate (3.26 g, 10.0 mmol) in EtOH:THF (5:1, 120 mL) was added palladium hydroxide on carbon (326 mg, 20% w/w). The reaction mixture was evacuated and placed under an atmosphere of H₂. This was repeated a further two times and the reaction allowed to stir under and atmosphere of H₂ for 16 hours The reaction mixture was filtered through Celite® and the filtrate was concentrated under reduced pressure to leave a pale yellow oil. Purification by column chromatography (5-10% MeOH/DCM) afforded the title intermediate as a thick colourless oil (1.34 g, 45% yield). ESMS: m/z 299 [M+H]⁺ and 597 [2M+Na]⁺. ¹H NMR (300 MHz, CDCl₃) δ: 5.27-5.19 (1H, m), 4.31-4.18 (1H, m), 3.75-3.63 (1H, m), 3.57-3.50 (2H, m), 3.31-3.22 (1H, m), 2.52-2.43 (1H, m) and 1.91-1.38 (15H, m).

### Intermediate 9

### Cyclopentyl 3-[1-(2-aminoethyl)piperidin-4-yl]-N-(tert-butoxycarbonyl)alaninate

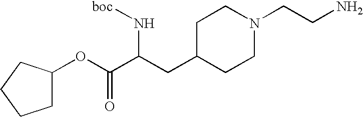

The title intermediate was prepared according to the procedure outlined below (Scheme 11).

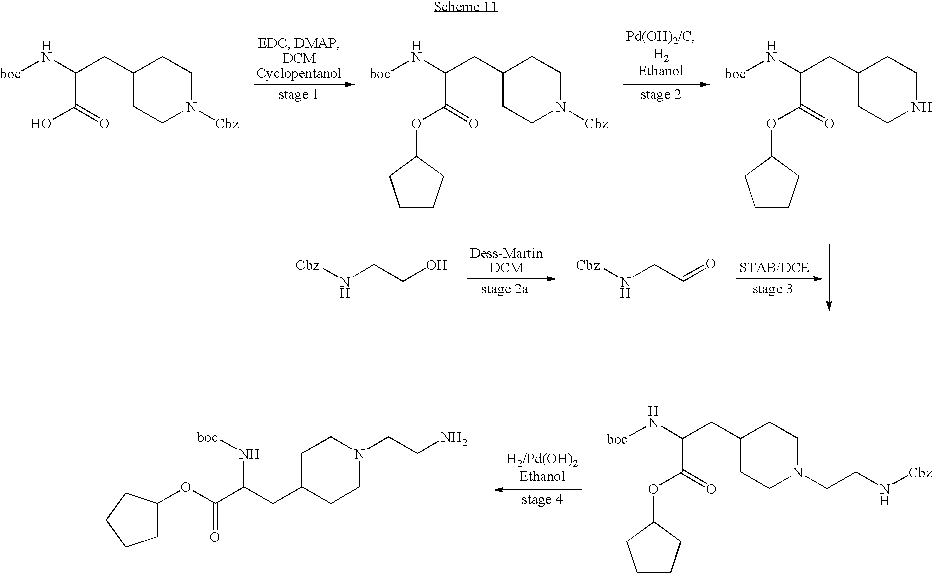

### Stage 1—Benzyl 4-{2-[(tert-butoxycarbonyl)amino]-3-(cyclopentyloxy)-3-oxopropyl}piperidine-1-carboxylate

To a solution of 3-{1-[(benzyloxy)carbonyl]piperidin-4-yl}-N-(tert-butoxycarbonyl)alanine (250 mg, 0.62 mmol) in DCM (5 mL) at 0° C. was added cyclopentanol (0.11 mL, 1.23 mmol), DMAP (9.6 mg, 0.06 mmol), and EDC (180 mg, 0.68 mmol). The reaction was allowed to warm to RT and stirred for a further 16 hours. The reaction mixture was diluted with water (30 mL) and EtOAc (30 mL). The aqueous layer was re-extracted with EtOAc (2×30 mL) and the combined organic layers washed with brine, dried (MgSO₄) and concentrated under reduced pressure to give crude product (340 mg, >100% yield) which was used without further purification. ESMS: m/z 475 [M+H]⁺.

### Stage 2—Cyclopentyl N-(tert-butoxycarbonyl)-3-piperidin-4-ylalaninate

To a solution of N-(tert-butoxycarbonyl)-3-piperidin-4-ylalanine (340 mg, 0.72 mmol) in ethanol (5 mL) was carefully added palladium hydroxide on carbon (68 mg, 20% w/w) under an atmosphere of nitrogen. The reaction mixture was evacuated and placed under an atmosphere of H₂. This was repeated a further two times and the reaction allowed to stir under and atmosphere of H₂ for 3 hours. The reaction mixture was filtered through Celite® and concentrated under reduced pressure to give the product (250 mg, >100% yield). ESMS: m/z 341 [M+H]⁺.

### Stage 2a—Benzyl (2-oxoethyl)carbamate

To a solution of benzyl (2-hydroxyethyl)carbamate (210 mg, 1.08 mmole) in DCM (3 mL) at −78° C. was added Dess-Martin periodinane (504 mg, 1.19 mmole). The reaction was allowed to warm to RT and stirred for a further 2 hours. The reaction was quenched by the addition of a saturated solution of 1:1 Na₂SO₃/NaHCO₃ (20 mL) and then extracted with DCM (3×30 mL). The combined organics were dried (MgSO₄) and concentrated under reduced pressure to give the desired product (150 mg, 70% yield) which required no further purification. ¹H NMR (300 MHz, CDCl₃) δ: 9.59 (1H, s), 7.28-7.30 (5H, m), 5.06 (2H, s) and 4.08 (2H, d, J=5.0 Hz).

### Stage 3—Cyclopentyl 3-[1-(2-{[(benzyloxy)carbonyl]amino}ethyl)piperidin-4-yl]-N-(tert-butoxycarbonyl)alaninate

To a solution of cyclopentyl N-(tert-butoxycarbonyl)-3-piperidin-4-ylalaninate (250 mg, 0.74 mmol) in DCE (5 mL) was added benzyl (2-oxoethyl)carbamate (131 mg, 0.67 mmol). The reaction was allowed to stir for 30 mins and then STAB (424 mg, 2.01 mmol) was added. The reaction was stirred for a further 16 hours and then quenched by the addition of sat. NaHCO₃ solution (10 ml). The mixture was extracted with DCM (3×30 mL), the organic layers combined, dried (MgSO₄) and concentrated under reduced pressure to give the product (240 mg, 69% yield). ESMS: m/z 518 [M+H]⁺.

### Stage 4—Cyclopentyl 3-[1-(2-aminoethyl)piperidin-4-yl]-N-(tert-butoxycarbonyl)alaninate

To a solution of cyclopentyl 3-[1-(2-{[(benzyloxy)carbonyl]amino}ethyl)piperidin-4-yl]-N-(tert-butoxycarbonyl)alaninate (240 mg, 0.46 mmol) in ethanol (5 mL) was carefully added palladium hydroxide on carbon (48 mg, 20% w/w) under an atmosphere of nitrogen. The reaction mixture was evacuated and placed under an atmosphere of H₂. This was repeated a further two times and the reaction allowed to stir under an atmosphere of H₂ for 3 hours. A further portion of palladium hydroxide on carbon (48 mg, 20% w/w) was added and the reaction stirred for an additional 16 hours. The reaction mixture was filtered through Celite® and concentrated under reduced pressure to give the title intermediate (250 mg). ESMS: m/z 384 [M+H]⁺.

### Intermediate 10

### Cyclopentyl O-[4-(aminomethyl)phenyl]-N-(tert-butoxycarbonyl)-L-homoserinate

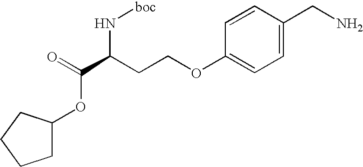

The title intermediate was prepared according to the procedure outlined below (Scheme 12).

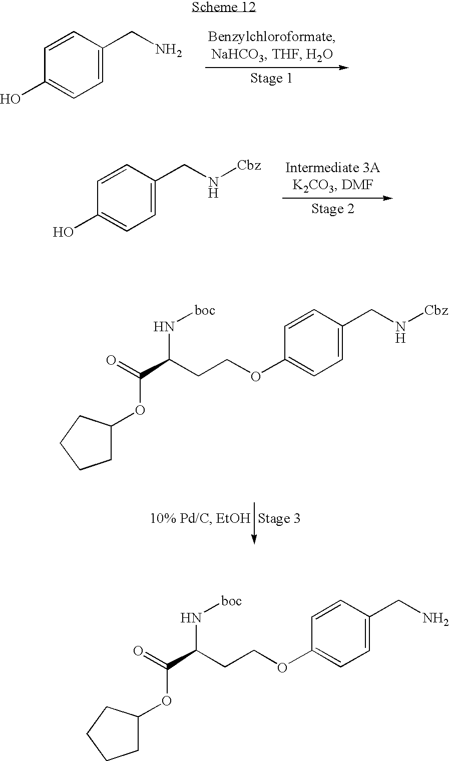

### Stage 1—Benzyl (4-hydroxybenzyl)carbamate

To a suspension of 4-(aminomethyl)phenol (300 mg, 2.44 mmol) in 10% THF/H₂O (10 mL) was added NaHCO₃ (266 mg, 3.17 mmol). The mixture was cooled to 0° C. and benzylchloroformate (344 μL, 2.44 mmol) added slowly. The reaction was stirred for 1.5 hours at RT. The reaction mixture was partitioned between water (40 mL) and EtOAc (40 mL). The organic layer was separated and the aqueous layer was re-extracted with EtOAc (20 mL). The combined organic layers were dried (MgSO₄) and concentrated under reduced pressure. The residue was triturated with heptane to afford the product as a white solid (610 mg, 97% yield). ESMS: m/z 258 [M+H]⁺

### Stage 2—(S)-4-[4-(Benzyloxycarbonylamino-methyl)-phenoxy]-2-tert butoxycarbonyl amino-butyric acid cyclopentyl ester

To a solution of benzyl (4-hydroxybenzyl)carbamate (150 mg, 0.58 mmol) in DMF (5 mL) was added potassium carbonate (107 mg, 0.77 mmol) and cyclopentyl (2S)-4-bromo-2-[(tert-butoxycarbonyl)amino]butanoate [intermediate 3A] (219 mg, 0.64 mmol). The reaction was heated for 20 hours at 60° C. The reaction mixture was concentrated under reduced pressure and then partitioned between water (30 mL) and EtOAc (30 mL). The aqueous layer was extracted with EtOAc (20 mL) and the combined organic layers were dried (MgSO₄) and concentrated under reduced pressure. The residue was purified by column chromatography (10-50% EtOAc/heptane) to afford the product (250 mg, 74% yield). ESMS: m/z 527 [M+H]⁺

### Stage 3—Cyclopentyl O-[4-(aminomethyl)phenyl]-N-(tert-butoxycarbonyl)-L-homoserinate

To a solution of (S)-4-[4-(Benzyloxycarbonylamino-methyl)-phenoxy]-2-tert-butoxycarbonylamino-butyric acid cyclopentyl ester (250 mg, 0.47 mmol) in ethanol (8 mL) was added a slurry of Pd/C (50 mg, 20% w/w) in EtOH (2 mL). The reaction was evacuated and put under a H₂ atmosphere for 2 hours. The reaction mixture was filtered through Celite® and washed with ethanol (15 mL). The filtrate was concentrated under reduced pressure to afford the title intermediate (110 mg, 59% yield). ESMS: m/z 393 [M+H]⁺. ¹H NMR (300 MHz, CDCl₃) δ: 7.21 (2H, d, 8.1 Hz), 6.84 (2H, d, J=8.4 Hz), 5.38 (1H, m), 5.22 (1H, m), 4.42 (1H, d, J=6.3 Hz), 4.03 (2H, t, 6 Hz), 3.80 (2H, s), 3.72 (1H, m), 2.29-1.51 (9H, m) 1.45 (9H, s), 1.28-1.20 (2H, m).

### Intermediate 11

### tert-Butyl O-[4-(aminomethyl)phenyl]-N-[(benzyloxy)carbonyl]-L-homoserinate

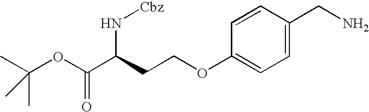

The title intermediate was prepared according to the procedure outlined below (Scheme 13).

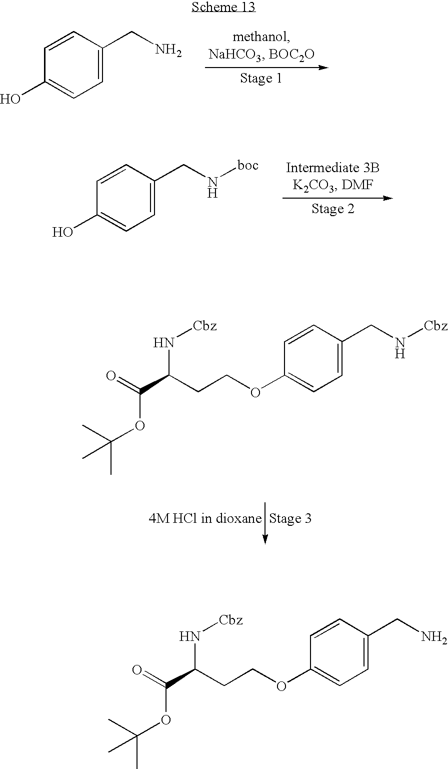

### Stage 1—tert-Butyl (4-hydroxybenzyl)carbamate

To a solution of 4-(aminomethyl)phenol (200 mg, 1.62 mmol) in MeOH (2.5 mL) was added sodium bicarbonate (476 mg, 5.68 mmol) and BOC₂O (390 mg, 1.79 mmol). The solution was stirred at RT for 72 hours. The reaction mixture was partitioned between water (20 mL) and EtOAc (20 mL). The organic layer was separated and the aqueous layer was extracted with EtOAc (10 mL). The combined organic layers were dried (MgSO₄) and concentrated under reduced pressure to give the product as a yellow oil (360 mg). ESMS: m/z 224 [M+H]⁺.

### Stage 2—tert-Butyl N-[(benzyloxy)carbonyl]-O-(4-{[(tert-butoxycarbonyl)amino]methyl}phenyl)-L-homoserinate

### Procedure as in Stage 2 Scheme 12 Using Intermediate 3B.

ESMS: m/z 515 [M+H]⁺.

### Stage 3—tert-Butyl O-[4-(aminomethyl)phenyl]-N-[(benzyloxy)carbonyl]-L-homoserinate

tert-butyl N-[(benzyloxy)carbonyl]-O-(4-{[(tert-butoxycarbonyl)amino]methyl}phenyl)-L-homoserinate (200 mg, 0.39 mmol) was dissolved in 4M HCl/dioxane (1.5 mL) and stirred at 0° C. for 20 minutes. The reaction mixture was filtered through Celite® and washed with ethanol (15 mL). The residue was diluted with EtOAc (15 mL) and the pH adjusted to 12 with 1M NaOH solution. The aqueous layer was extracted with EtOAc (3×10 mL) and the combined organics were dried (MgSO₄) and concentrated under reduced pressure to afford the title intermediate as a colourless oil (152 mg, 95% yield). ESMS: m/z 224 [M+H]⁺. ¹H NMR (300 MHz, CDCl₃) δ: 7.36 (5H, s), 7.19 (2H, d, J=8.5 Hz), 6.83 (2H, d, J=8.3 Hz), 5.12 (2H, s), 4.45 (1H, br. s.), 4.25 (2H, d, J=5.3 Hz), 4.04 (2H, t, J=6.0 Hz), 2.11-2.46 (2H, m) and 1.48 (9H, s).

### Intermediate 12A

### 1-Benzyl 2-cyclopentyl piperazine-1,2-dicarboxylate

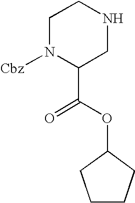

The title intermediate was prepared according to the procedure outlined below (Scheme 14).

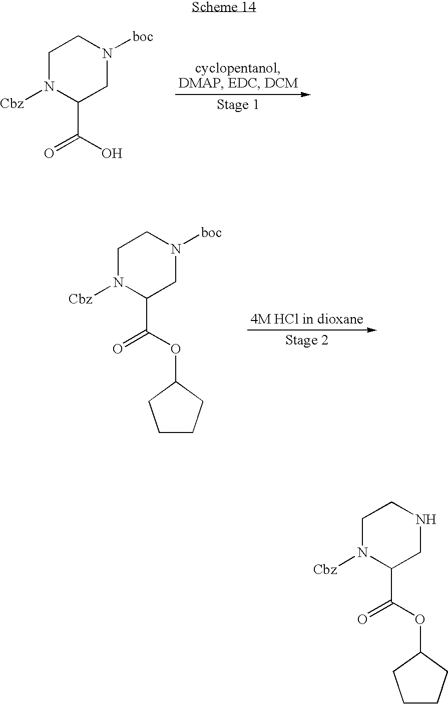

### Stage 1—1-Benzyl 4-tert-butyl 2-cyclopentyl piperazine-1,2,4-tricarboxylate

To a solution of 1-[(benzyloxy)carbonyl]-4-(tert-butoxycarbonyl)piperazine-2-carboxylic acid (1.00 g, 2.85 mmol) in DCM (20 mL) at 0° C. was added cyclopentanol (520 μL 5.70 mmol), EDC (602 mg, 3.14 mmol) and DMAP (35 mg, 0.29 mmol). The reaction mixture was stirred for 48 hours at RT then the solvent removed under reduced pressure. The crude residue was dissolved in EtOAc (30 mL) and washed with 1M HCl (15 mL), 1M Na₂CO₃ (15 mL) and brine (10 mL). The organic layer was dried (MgSO₄) and the solvent removed under reduced pressure to give the product (1.23 g, 95% yield). ESMS: m/z 433 [M+H]⁺.

### Stage 2—1-Benzyl 2-cyclopentyl piperazine-1,2-dicarboxylate

1-Benzyl 2-cyclopentyl piperazine-1,2-dicarboxylate (200 mg, 0.39 mmol) was dissolved in 4M HCl/dioxane (3 mL) and stirred at 0° C. for 1 hour. The reaction mixture was concentrated under reduced pressure to afford the title intermediate as a colourless oil (145 mg). ESMS: m/z 333 [M+H]⁺. ¹H NMR (300 MHz, CDCl₃) δ: 7.20-7.28 (5H, m), 5.16-5.17 (1H, m), 5.01-5.09 (2H, m), 4.49-4.60 (1H, m), 3.82 (1H, t J=14.8 Hz), 3.43 (1H, t J=12.9 Hz), 2.26-3.12 (4H, m) and 1.51-1.76 (8H, m).

### Intermediate 12B

### 1-Benzyl 2-cyclopentyl 4-(2-aminoethyl)piperazine-1,2-dicarboxylate

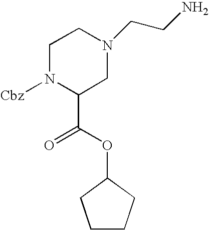

The title intermediate was prepared according to the procedure outlined below (Scheme 15).

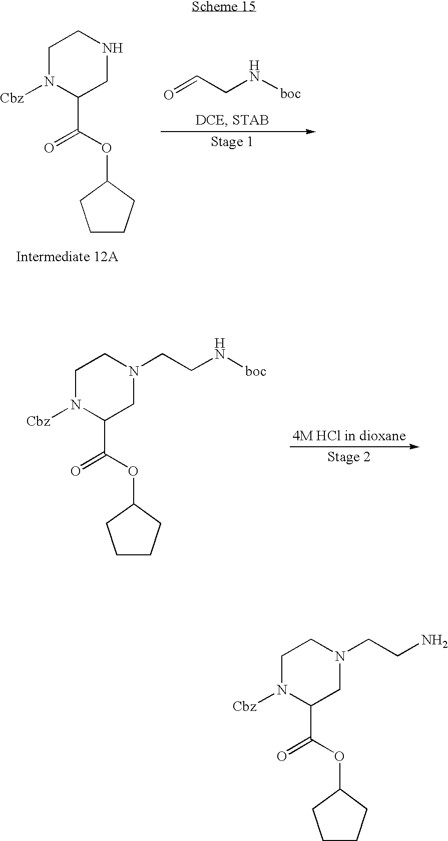

### Stage 1—1-tert-Butyl 2-cyclopentyl 4-{2-[(tert-butoxycarbonyl)amino]ethyl}piperazine-1,2-dicarboxylate

To a solution of 1-benzyl 2-cyclopentyl piperazine-1,2-dicarboxylate [Intermediate 12A] (165 mg, 0.50 mmol) in DCE (8 mL) was added the tert-butyl (2-oxoethyl)carbamate (72 mg, 0.45 mmol). After stirring at RT for 10 minutes AcOH (35 μL) and STAB (287 mg, 1.35 mmol) were added. After stirring for 1 hour the mixture was quenched with sat NaHCO₃ (2 mL) and diluted with DCM (10 mL). The organic layer was washed with 1M HCl (10 mL), 1M Na₂CO₃ (10 mL) and brine (10 mL), dried (MgSO₄) and evaporated under reduced pressure to isolate the crude product (240 mg). ESMS: m/z 476 [M+H]⁺.

### Stage 2—1-Benzyl 2-cyclopentyl 4-(2-aminoethyl)piperazine-1,2-dicarboxylate

### Procedure as in [Scheme 14 Stage 2].

ESMS: m/z 376 [M+H]⁺. ¹H NMR (300 MHz, CDCl₃) δ: 7.17-7.31 (5H, m), 4.98-5.20 (3H, m), 4.44-4.91 (2H, m), 3.83 (1H, t, J=14.8 Hz), 3.04-3.52 (4H, m), 1.91-2.46 (4H, m) and 1.44-1.85 (8H, m).

### Intermediate 12C

### 1-Benzyl 2-tert-butyl 4-(2-aminoethyl)piperazine-1,2-dicarboxylate

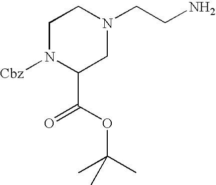

The title intermediate was prepared according to the procedure outlined below (Scheme 16).

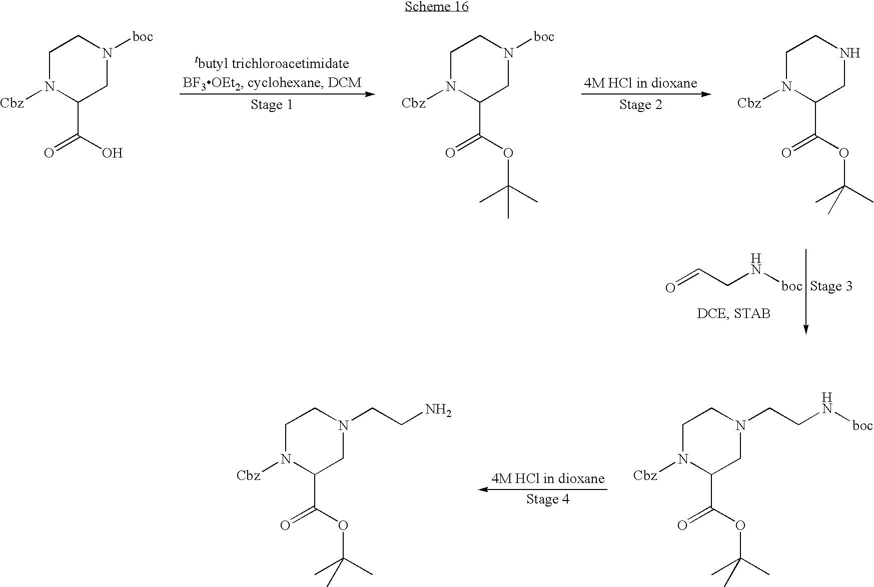

### Stage 1—1-Benzyl 2,4-di-tert-butyl piperazine-1,2,4-tricarboxylate

To a solution of 1-[(benzyloxy)carbonyl]-4-(tert-butoxycarbonyl)piperazine-2-carboxylic acid (500 mg, 1.37 mmol) in DCM (10 mL) and cyclohexane (10 mL) at 0° C. was added boron trifluoride triethyl etherate followed immediately by slow addition of butyl trichloroacetimidate (600 mg, 2.74 mmol) in cyclohexane (10 mL) over 15 min. The reaction was allowed to warm to RT and stirred for 30 min. Sodium hydrogen carbonate (80 mg) was added, and stirring continued for a further 10 minutes before filtering through Celite®. The Celite® was washed thoroughly with DCM and the filtrate solvent removed under reduced pressure. The residue was purified by column chromatography (10% EtOAc/heptane) to afford the product as a white solid (0.240 g, 42% yield). ESMS: m/z 443 [M+H]⁺.

### Stage 2—1-Benzyl 2-tert-butyl piperazine-1,2-dicarboxylate

1-Benzyl 2,4-di-tert-butyl piperazine-1,2,4-tricarboxylate (240 mg, 0.57 mmol) was dissolved in 4M HCl/dioxane (1.5 mL) and stirred at RT for 1 hour. The mixture was diluted in EtOAc (10 mL) and washed in 2M NaOH. The organic layer was then dried (MgSO₄) and evaporated under reduced pressure to give the crude product (240 mg). ESMS: m/z 321 [M+H]⁺.

### Stage 3—1-Benzyl 2-tert-butyl 4-{2-[(tert-butoxycarbonyl)amino]ethyl}piperazine-1,2-dicarboxylate

Procedure as in [Scheme 15 Stage 1]

ESMS: m/z 464 [M+H]⁺.

### Stage 4—1-Benzyl 2-tert-butyl 4-(2-aminoethyl)piperazine-1,2-dicarboxylate

Procedure as in [Scheme 13 Stage 3]

ESMS: m/z 364 [M+H]⁺. ¹H NMR (300 MHz, CDCl₃) δ: 7.17-7.31 (5H, m), 4.98-5.20 (3H, m), 4.44-4.91 (2H, m), 3.83 (1H, t, J=14.8 Hz), 3.04-3.52 (4H, m), 1.91-2.46 (4H, m) and 1.35 (9H, s).

### Intermediate 13

### Cyclopentyl (2S)-2-[(tert-butoxycarbonyl)amino]pent-4-enoate

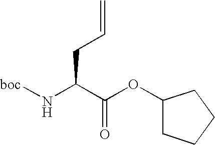

The title intermediate was prepared according to the procedure outlined below (Scheme 17).

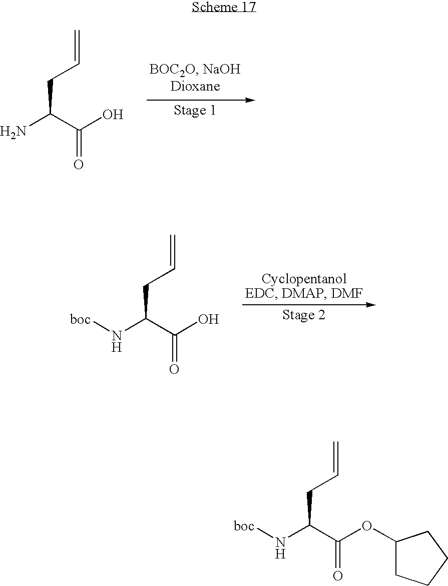

### Stage 1—(2S)-2-[(tert-Butoxycarbonyl)amino]pent-4-enoic acid

To a solution of (2S)-2-aminopent-4-enoic acid (1.00 g, 8.70 mmol) in 1M NaOH (20 mL) and dioxane (10 mL) at 0° C. was added BOC₂O (2.28 g, 10.5 mmol). The reaction mixture was allowed to warm to RT and stirred for an additional 18 hours. The pH was checked and adjusted to basic when necessary. The reaction mixture was concentrated under reduced pressure and the aqueous phase washed with Et₂O (2×10 mL) to remove the excess BOC₂O. The aqueous phase was acidified to pH2 with 2 M H₂SO₄ and extracted with EtOAc (4×20 mL) while saturating the aqueous each time with sodium chloride. The combined organic layers were dried (MgSO₄) and concentrated under reduced pressure to afford the product (2.2 g, 100% yield). ESMS m/z: 238 [M+Na]⁺.

### Stage 2—Cyclopentyl (2S)-2-[(tert-butoxycarbonyl)amino]pent-4-enoate

To a solution of (S)-2-tert-butoxycarbonylamino-pent-4-enoic acid (2.20 g, 10.2 mmol) in DCM (50 mL) was added DMAP (125 mg, 1.02 mmol), cyclopentanol (1.1 mL, 12.2 mmol) and EDC (2.15 g, 11.2 mmol). The reaction was stirred for 65 hours and concentrated under reduced pressure. Purification by column chromatography (5% EtOAc/heptane) afforded the titled intermediate as a clear oil (1.75 g, 60% yield). ¹H NMR (300 MHz, CDCl₃) δ: 5.61-5.79 (1H, m), 5.21 (1H, dd, J=8.3, 3.4 Hz), 5.15 (1H, dd, J=2.9, 1.2 Hz), 5.10 (1H, d, J=1.3 Hz), 4.25-4.38 (1H, m), 2.49 (1H, dd, J=12.8, 6.4 Hz), 1.53-1.92 (8H, m) and 1.44 (9H, s).

### Example 1

### Cyclopentyl 4-{[(7R)-8-cyclopentyl-7-ethyl-5-methyl-6-oxo-5,6,7,8-tetrahydropteridin-2-yl]amino}-L-phenylalaninate

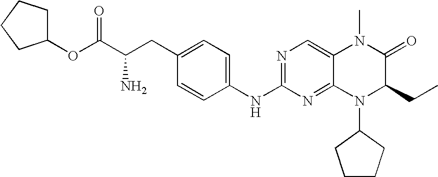

The titled example was prepared according to the procedure outlined below (Scheme 18).

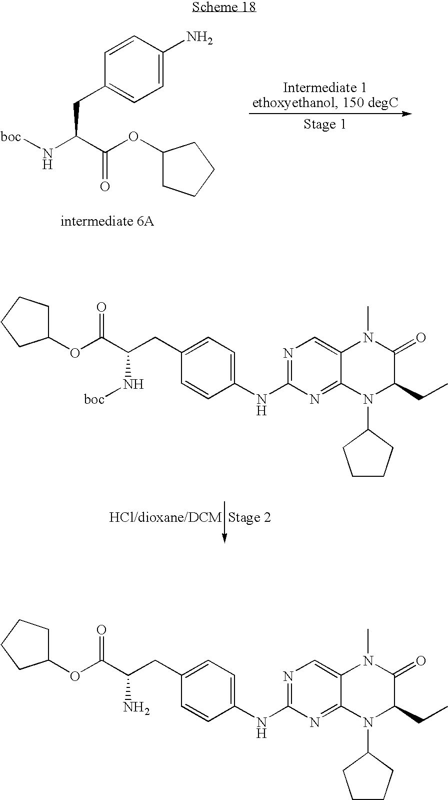

### Stage 1—Cyclopentyl N-(tert-butoxycarbonyl)-4-{[(7R)-8-cyclopentyl-7-ethyl-5-methyl-6-oxo-5,6,7,8-tetrahydropteridin-2-yl]amino}-L-phenylalaninate

To a solution of (7R)-2-chloro-8-cyclopentyl-7-ethyl-5-methyl-7,8-dihydropteridin-6(5H)-one [Intermediate 1] (100 mg, 0.34 mmol) in 2-ethoxyethanol (2 mL) was added cyclopentyl 4-amino-N-(tert-butoxycarbonyl)-L-phenylalaninate [Intermediate 6A] (170 mg, 0.51 mmol). The reaction mixture was heated at 150° C. for 4 hours, cooled and concentrated under reduced pressure to give a brown residue. The residue was purified by column chromatography (5% methanol/1% NH₄OH in EtOAc) to afford the product as a yellow solid (89 mg, 43% yield). ESMS: m/z 607 [M+H]⁺.

### Stage 2—Cyclopentyl 4-{[(7R)-8-cyclopentyl-7-ethyl-5-methyl-6-oxo-5,6,7,8-tetrahydropteridin-2-yl]amino}-L-phenylalaninate

To a solution of (S)-2-tert-butoxycarbonylamino-3-[4((R)-8-cyclopentyl-7-ethyl-5-methyl-6-oxo-5,6,7,8-tetrahydropteridin-2-yl]amino}-L-phenylalaninate (32 mg, 0.05 mmol) in DCM (3 mL) was added 4M HCl/dioxane (3 mL). The reaction mixture was stirred at RT for 4 hours and concentrated under reduced pressure to give a brown residue. The pH of the residue was adjusted to 9 with saturated NaHCO₃ solution (3 mL) and then extracted with EtOAc (3×10 mL). The combined organics were dried (MgSO₄) and concentrated under reduced pressure to afford the title example as a white solid (9 mg, 34% yield). ESMS: m/z 507 [M+H]⁺. ¹H NMR (300 MHz, CDCl₃) δ: 7.43 (2H, d, J=7.7 Hz), 7.04-7.33 (3H, m), 5.06-5.24 (1H, m), 4.02-4.18 (1H, m), 4.20-4.44 (2H, m), 3.14 (2H, m), 2.83-11.04 (1H, s), 1.32-2.14 (18H, m) and 0.80 (3H, t, J=7.4 Hz).

### Example 2

### Cyclopentyl (2S,4E)-2-amino-5-(4-{[7R)-8-cyclopentyl-7-ethyl-5-methyl-6-oxo-5,6,7,8-tetrahydropteridin-2-yl]amino}phenyl)pent-4-enoate

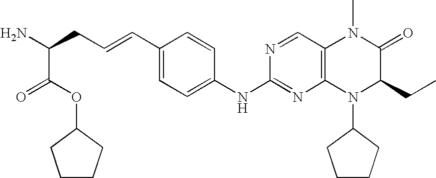

The title example was prepared according to the procedure outlined below (Scheme 19):

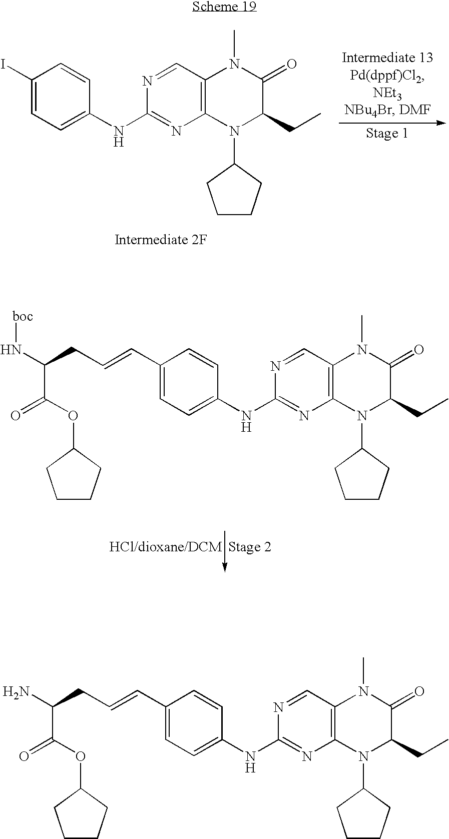

### Stage 1—(7R)-8-Cyclopentyl-7-ethyl-2-[(4-iodophenyl)amino]-5-methyl-7,8-dihydropteridin-6(5H)-one

To a solution of cyclopentyl (2S)-2-[(tert-butoxycarbonyl)amino]pent-4-enoate [Intermediate 13] (175 mg, 0.62 mmol) in DMF (3 mL) was added (7R)-8-cyclopentyl-7-ethyl-2-[(4-iodo-phenyl)amino]-5-methyl-7,8-dihydro pteridin-6(5H)-one [Intermediate 2F] (197 mg, 0.41 mmol), Pd(dppf)Cl₂ (34 mg, 0.04 mmol), Et₃N (0.13 mL, 0.90 mmol) and NBu₄Br (133 mg, 0.40 mmol). The reaction mixture was heated at 120° C. for 1 h in the microwave and concentrated under reduced pressure. The crude residue was absorbed onto silica and purified by column chromatography (40% EtOAc/heptane) to give the product (72 mg, 30% yield). ESMS m/z: 633 [M+H]⁺.

### Stage 2—Cyclopentyl (2S,4E)-2-[(tert-butoxycarbonyl)amino]-5-(4-{[(7R)-8-cyclopentyl-7-ethyl-5-methyl-6-oxo-5,6,7,8-tetrahydropteridin-2-yl]amino}phenyl)pent-4-enoate

To a solution of (7R)-8-Cyclopentyl-7-ethyl-2-[(4-iodophenyl)amino]-5-methyl-7,8-dihydropteridin-6(5H)-one (36 mg, 0.06 mmol) in DCM (2 mL) was added 4M HCl/dioxane (20 μl, 0.08 mmol). The reaction mixture was stirred at RT for 2 h and then concentrated under reduced pressure. The residue was redissolved in DCM (10 ml) washed with 1M NaHCO₃ (10 mL), dried (MgSO₄) and evaporated under reduced pressure. Purification by reverse phase chromatography afforded the title example as a yellow oil (3 mg, 10% yield). ESMS m/z: 533 [M+H]⁺. ¹H NMR (300 MHz, MeOD) δ: 7.55-7.62 (1H, m), 7.39-7.51 (4H, m), 6.61 (1H, d, J=15.6 Hz), 6.17 (1H, ddd, J=15.4, 7.6, 7.3 Hz), 5.26-5.34 (1H, m), 4.39 (1H, dd, J=6.3, 3.3 Hz), 4.32 (1H, t, J=8.8 Hz), 4.17 (1H, t, J=6.2 Hz), 3.25 (3H, s), 2.83 (2H, t, J=6.7 Hz), 1.79-2.06 (9H, m), 1.54-1.78 (9H, m) and 0.86 (3H, t, J=7.4 Hz)

### Example 3

### Cyclopentyl O-(4-{[(7R)-8-cyclopentyl-7-ethyl-5-methyl-6-oxo-5,6,7,8-tetrahydropteridin-2-yl]amino}phenyl)-L-homoserinate

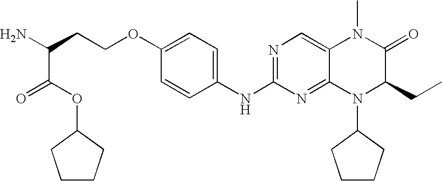

The titled example was prepared according to the general procedure outlined below (Scheme 20).

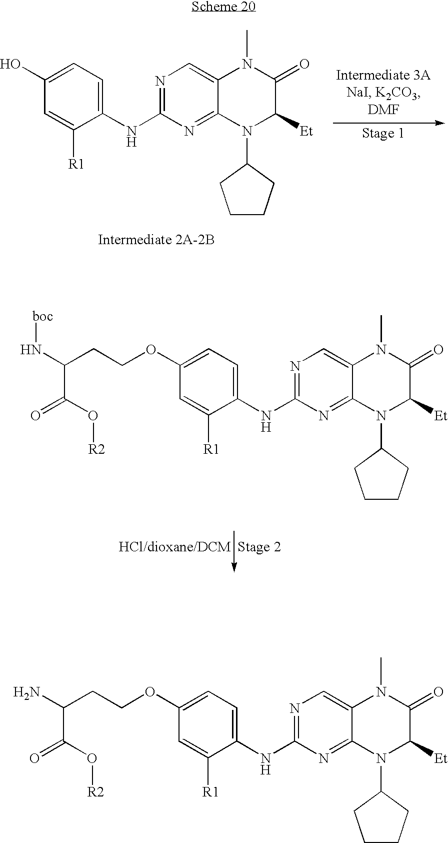

### Stage 1—Cyclopentyl N-(tert-butoxycarbonyl)-O-(4-{[(7R)-8-cyclopentyl-7-ethyl-5-methyl-6-oxo-5,6,7,8-tetrahydropteridin-2-yl]amino}phenyl)-L-homoserinate

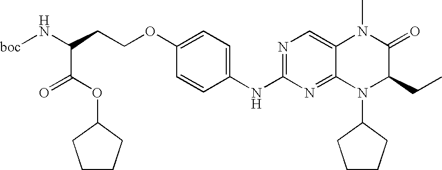

To a solution of (7R)-8-cyclopentyl-7-ethyl-2-[(4-hydroxyphenyl)amino]-5-methyl-7,8-dihydropteridin-6(5H)-one [Intermediate 2A] (120 mg, 0.33 mmol) in DMF (2 mL) was added cyclopentyl (2S)-4-bromo-2-[(tert-butoxycarbonyl)amino]butanoate [Intermediate 3A] (114 mg, 0.33 mmol) and K₂CO₃ (90 mg, 0.65 mmol). The reaction mixture was stirred for 40 hours at 40° C. and then the reaction mixture was diluted with EtOAc (25 mL). The mixture was washed with water (2×25 mL) and brine (25 mL). The organic layer was dried (MgSO₄) and concentrated under reduced pressure to leave a brown oil. Purification by column chromatography (100% EtOAc) afforded the product as a pale brown solid (177 mg, 85% yield). ESMS: m/z 637 [M+H]⁺.

### Stage 2—Cyclopentyl O-(4-{[(7R)-8-cyclopentyl-7-ethyl-5-methyl-6-oxo-5,6,7,8-tetrahydropteridin-2-yl]amino}phenyl)-L-homoserinate

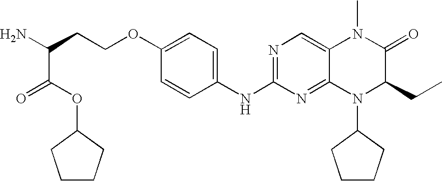

Cyclopentyl N-(tert-butoxycarbonyl)-O-(4-{[(7R)-8-cyclopentyl-7-ethyl-5-methyl-6-oxo-5,6,7,8-tetrahydropteridin-2-yl]amino}phenyl)-L-homoserinate (177 mg, 0.28 mmol) was suspended in a solution of 4M HCl/dioxane (2 mL). The reaction mixture was stirred at RT for 30 minutes and concentrated under reduced pressure to leave a thick yellow oil. Trituration with Et₂O afforded an off-white solid, which was partitioned between DCM (25 mL) and sat. Na₂CO₃ (25 mL). The organic layer was separated, dried (MgSO₄) and concentrated under reduced pressure to afford the title example as an off-white solid (90 mg, 60% yield). ESMS: m/z 537 [M+H]⁺. ¹H NMR (300 MHz, MeOD) δ: 7.55 (1H, s), 7.32 (2H, d, J=9.0 Hz), 6.76 (2H, d, J=9.0 Hz), 5.15-5.09 (1H, m), 4.28-4.19 (1H, m), 4.11 (1H, dd, J=3.6, 7.5 Hz), 4.01-3.95 (2H, m), 3.55 (1H, t, J=6.5 Hz), 3.20 (3H, s), 2.11-1.51 (20H, m) and 0.75 (3H, t, J=7.5 Hz).

The example in the following table was prepared by methods analogous to the method described above (Scheme 20) using the appropriate intermediates.

| Example | Intermediates Used | Name | ESMS |
|---|---|---|---|
| 4 | 2B & 3A | Cyclopentyl O-(4-{[(7R)-8-cyclopentyl-7- | m/z |
| | | ethyl-5-methyl-6-oxo-5,6,7,8-tetrahydropteridin- | 567 [M + H]⁺ |
| | | 2-yl]amino}-3-methoxyphenyl)-L- | |
| | | homoserinate | |

### Example 5

### Cyclopentyl (2S)-2-amino-4-[(4-{[(7R)-8-cyclopentyl-7-ethyl-5-methyl-6-oxo-5,6,7,8-tetrahydropteridin-2-yl]amino}-3-methoxybenzoyl)amino]butanoate

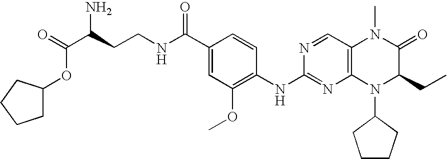

The titled example was prepared according to the general procedure outlined below (Scheme 21).

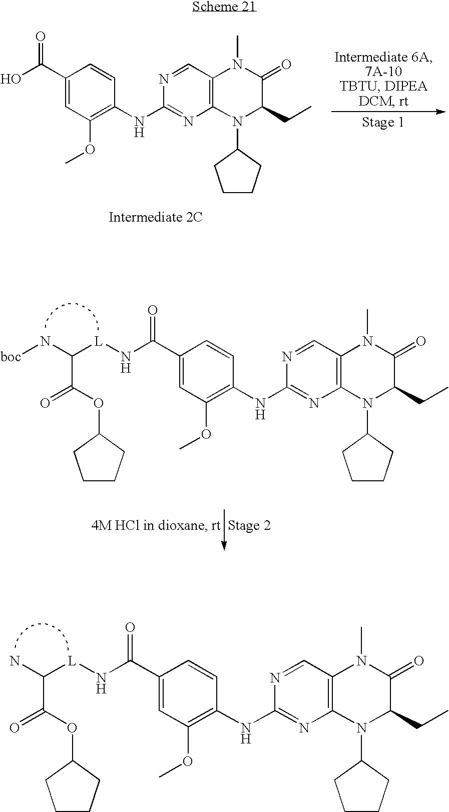

### Stage 1—Cyclopentyl (2S)-2-[(tert-butoxycarbonyl)amino]-4-[(4-{[(7R)-8-cyclopentyl-7-ethyl-5-methyl-6-oxo-5,6,7,8-tetrahydropteridin-2-yl]amino}-3-methoxybenzoyl)amino]butanoate

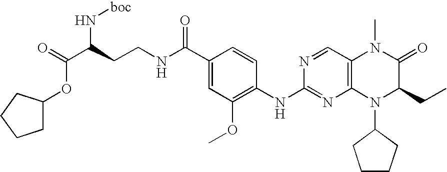

To a solution of 4-{[(7R)-8-cyclopentyl-7-ethyl-5-methyl-6-oxo-5,6,7,8-tetrahydropteridin-2-yl]amino}-3-methoxybenzoic acid [Intermediate 2C] (200 mg, 0.47 mmol) in DCM (5 mL) was added O-benzotriazol-1-yl-N,N,N′,N′-tetramethyluronium tetrafluoroborate (166 mg, 0.52 mmol) and DIPEA (0.16 mL, 0.94 mmol). The reaction mixture was stirred at RT for 30 minutes before adding cyclopentyl (2S)-4-amino-2-[(tert-butoxycarbonyl)amino]butanoate [Intermediate 7A] (269 mg, 0.84 mmol). The reaction mixture was stirred at RT for a further 18 hours then diluted with DCM (20 mL), and washed with water (2×20 mL). The organic layer was dried (MgSO₄) and concentrated under reduced pressure to leave a yellow oil. Purification by column chromatography (100% EtOAc) afforded the product as a yellow solid (228 mg, 70% yield). ESMS: m/z 694 [M+H]⁺.

### Stage 2—Cyclopentyl (2S)-2-amino-4-[(4-{[(7R)-8-cyclopentyl-7-ethyl-5-methyl-6-oxo-5,6,7,8-tetrahydropteridin-2-yl]amino}-3-methoxy benzoyl)amino]butanoate

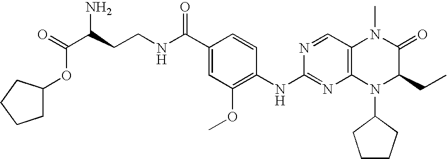

Cyclopentyl (2S)-2-[(tert-butoxycarbonyl)amino]-4-[(4-{[(7R)-8-cyclopentyl-7-ethyl-5-methyl-6-oxo-5,6,7,8-tetrahydropteridin-2-yl]amino}-3-methoxy benzoyl)amino]butanoate (228 mg, 0.33 mmol) was dissolved in DCM (20 mL) and 4M HCl/dioxane (10 mL) was added. The reaction mixture was stirred at RT for 2 hours and concentrated under reduced pressure. The residue was taken up in EtOAc (50 mL), washed with sat. Na₂CO₃ (25 mL), brine (25 mL). The organic layer was dried (MgSO₄) and concentrated under reduced pressure to afford the title example as a white solid (180 mg, 92% yield). ESMS: m/z 594 [M+H]⁺. ¹H NMR (300 MHz, CDCl₃) δ: 8.55 (1H, d, J=8.4 Hz), 7.70 (2H, br s), 7.62 (1H, s), 7.48 (1H, d, J=1.5 Hz), 7.32 (1H, dd, J=2.0, 8.6 Hz), 5.23-5.19 (1H, m), 4.55-4.49 (1H, m), 4.24 (1H, dd, J=3.6, 7.8 Hz), 3.99 (3H, s), 3.92-3.80 (1H, m), 3.59-3.47 (2H, m), 3.35 (3H, s), 2.14-1.60 (22H, m) and 0.90 (3H, t, J=7.4 Hz).

The examples in the following table were prepared by methods analogous to the method described above (Scheme 21) using the appropriate intermediates.

| | Stage 1 | | |
|---|---|---|---|
| | Intermediates | | |
| Example | used | Name | ESMS |
| 6 | 2C & 8 | Cyclopentyl (4S)-4-[(4-{[(7R)-8-cyclopentyl-7-ethyl-5- | m/z |
| | | methyl-6-oxo-5,6,7,8-tetrahydropteridin-2-yl]amino}-3- | 606 [M + H]⁺ |
| | | methoxybenzoyl)amino]-L-prolinate | |
| 7 | 2C & 6A | Cyclopentyl 4-[(4-{[(7R)-8-cyclopentyl-7-ethyl-5-methyl- | m/z |
| | | 6-oxo-5,6,7,8-tetrahydropteridin-2-yl]amino}-3- | 656 [M + H]+ |
| | | methoxybenzoyl)amino]-L-phenylalaninate | |
| 8 | 2C & 7B | Cyclopentyl N⁶-(4-{[(7R)-8-cyclopentyl-7-ethyl-5-methyl- | m/z |
| | | 6-oxo-5,6,7,8-tetrahydropteridin-2-yl]amino}-3- | 622 [M + H]+ |
| | | methoxybenzoyl)lysinate | |
| 9 | 2C & 10 | Cyclopentyl O-(4-{[(4-{[(7R)-8-cyclopentyl-7-ethyl-5- | m/z |
| | | methyl-6-oxo-5,6,7,8-tetrahydropteridin-2-yl]amino}-3- | 700 [M + H]⁺ |
| | | methoxybenzoyl)amino]methyl}phenyl)-L-homoserinate | |
| 10 | 2C & 9 | Cyclopentyl 3-(1-{2-[(4-{[(7R)-8-cyclopentyl-7-ethyl-5- | m/z |
| | | methyl-6-oxo-5,6,7,8-tetrahydropteridin-2-yl]amino}-3- | 691 [M + H]⁺ |
| | | methoxybenzoyl)amino]ethyl}piperidin-4-yl)alaninate | |
| *11 | 2C & 6B | tert-Butyl 4-[(4-{[(7R)-8-cyclopentyl-7-ethyl-5-methyl-6- | m/z |
| | | oxo-5,6,7,8-tetrahydropteridin-2-yl]amino}-3- | 664 [M + H]⁺ |
| | | methoxybenzoyl)amino]-L-phenylalaninate | |

*In order to achieve selective Boc deprotection [Scheme 21 Stage 2] the mixture was stirred at 0° C. for 30 minutes instead of RT for 2 hours.

### Example 12

### tert-Butyl O-(4-{[4-{[7R)-8-cyclopentyl-7-ethyl-5-methyl-6-oxo-5,6,7,8-tetrahydro pteridin-2-yl]amino}-3-methoxybenzoyl)amino]methyl}phenyl)-L-homoserinate

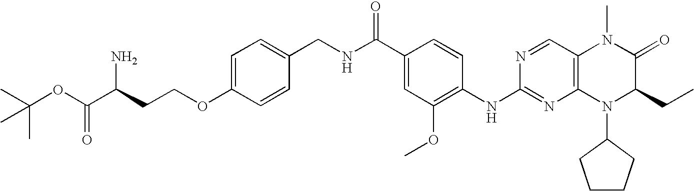

The titled example was prepared according to the general procedure outlined below (Scheme 22).

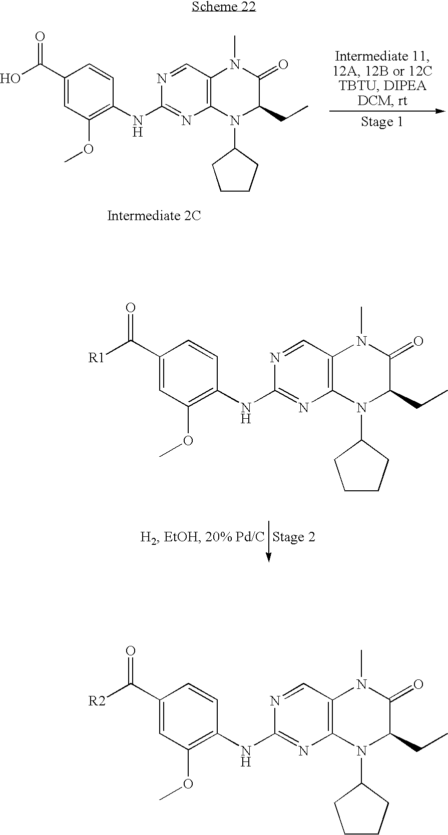

### Stage 1—tert-Butyl N-[(benzyloxy)carbonyl]-O-(4-{[(4-{[(7R)-8-cyclopentyl-7-ethyl-5-methyl-6-oxo-5,6,7,8-tetrahydropteridin-2-yl]amino}-3-methoxybenzoyl)amino]-methyl}phenyl)-L-homoserinate

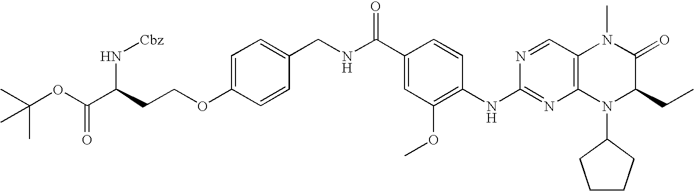

Procedure as in [Scheme 21 Stage 1] using intermediates 2C and 11.

### Stage 2—tert-Butyl O-(4-{[(4-{[(7R)-8-cyclopentyl-7-ethyl-5-methyl-6-oxo-5,6,7,8-tetrahydropteridin-2-yl]amino}-3-methoxybenzoyl)amino]methyl}phenyl)-L-homoserinate

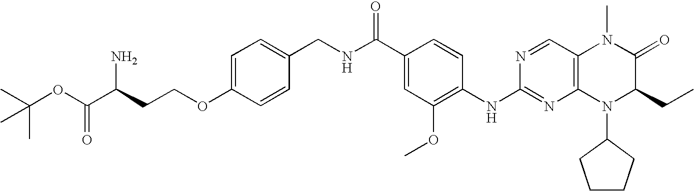

To a solution of tert-butyl N-[(benzyloxy)carbonyl]-O-(4-{[(4-{[(7R)-8-cyclopentyl-7-ethyl-5-methyl-6-oxo-5,6,7,8-tetrahydropteridin-2-yl]amino}-3-methoxybenzoyl)amino]-methyl}phenyl)-L-homoserinate (132 mg, 0.16 mmol) in EtOH (5 mL) under a nitrogen atmosphere was added Pd/C (30 mg, 20% w/w). The reaction mixture was evacuated and placed under an atmosphere of H₂. This was repeated a further two times and the reaction allowed to stir under an atmosphere of H₂ for 1 hour. The reaction mixture was filtered through Celite® and the filtrate concentrated under reduced pressure and purified by column chromatography (10% MeOH/DCM) to yield the titled example as a white solid (42 mg, 38% yield). ESMS: m/z 688 [M+H]⁺. ¹H NMR (300 MHz, CDCl₃) δ: 8.52 (1H, d, J=8.5 Hz), 7.56-7.70 (2H, m), 7.48 (1H, d, J=1.3 Hz), 7.27-7.32 (2H, m), 6.88 (2H, d, J=8.5 Hz), 6.53 (1H, t, J=5.5 Hz), 4.44-4.62 (3H, m), 4.21 (1H, dd, J=7.7, 3.6 Hz), 3.95 (3H, s), 3.59 (1H, dd, J=7.6, 5.0 Hz), 3.31 (3H, s), 2.08-2.28 (4H, m), 1.66-2.01 (10H, m), 1.47 (9H, m) and 0.87 (3H, t, J=7.5 Hz).

The examples in the following table were prepared by methods analogous to the method described above (Scheme 22) using the appropriate intermediates.

| | Stage 1 | | |
|---|---|---|---|
| | Intermediates | | |
| Example | used | Name | ESMS |
| 13 | 2C & 12A | Cyclopentyl 4-(4-{[(7R)-8-cyclopentyl-7-ethyl- | m/z |
| | | 5-methyl-6-oxo-5,6,7,8-tetrahydropteridin-2- | 606 [M + H]⁺ |
| | | yl]amino}-3-methoxybenzoyl)piperazine-2- | |
| | | carboxylate | |
| 14 | 2C & 12B | Cyclopentyl 4-{2-[(4-{[(7R)-8-cyclopentyl-7- | m/z |
| | | ethyl-5-methyl-6-oxo-5,6,7,8-tetrahydropteridin- | 325 [(M + 2)/2]⁺ |
| | | 2-yl]amino}-3-methoxybenzoyl)amino]ethyl}piperazine-2- | |
| | | carboxylate | |
| 15 | 2C & 12C | tert-butyl 4-{2-[(4-{[(7R)-8-cyclopentyl-7- | m/z |
| | | ethyl-5-methyl-6-oxo-5,6,7,8-tetrahydropteridin- | 637 [M + H]⁺ |
| | | 2-yl]amino}-3-methoxybenzoyl)amino]ethyl}piperazine-2- | |
| | | carboxylate | |

### Example 16

### Cyclopentyl (2S)-2-amino-4-{3-[(4-{[7R)-8-cyclopentyl-7-ethyl-5-methyl-6-oxo-5,6,7,8-tetrahydropteridin-2-yl]amino}-3-methoxybenzoyl)amino]pyrrolidin-1-yl}butanoate

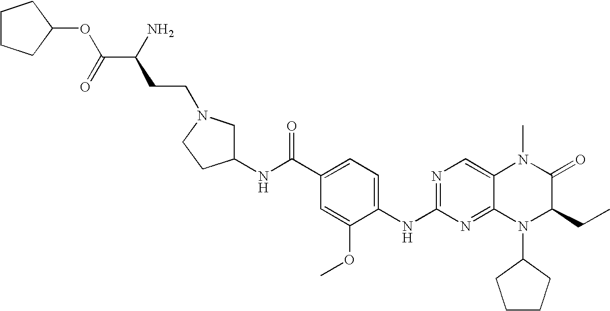

The titled example was prepared according to the general procedure outlined below (Scheme 23).

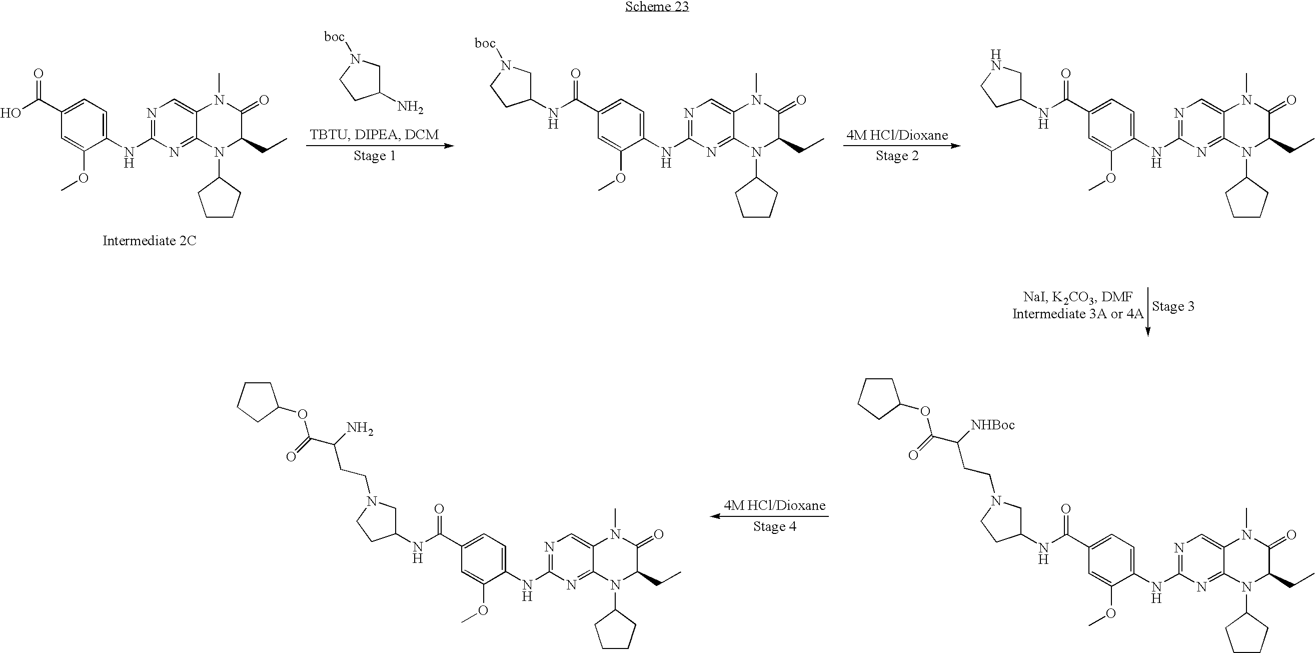

### Stage 1—tert-Butyl 3-[(4-{[(7R)-8-cyclopentyl-7-ethyl-5-methyl-6-oxo-5,6,7,8-tetrahydropteridin-2-yl]amino}-3-methoxybenzoyl)amino]pyrrolidine-1-carboxylate

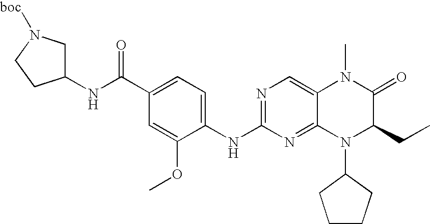

To a solution of 4-{[(7R)-8-cyclopentyl-7-ethyl-5-methyl-6-oxo-5,6,7,8-tetrahydropteridin-2-yl]amino}-3-methoxybenzoic acid [Intermediate 2C] (200 mg, 0.47 mmol) in DCM (10 mL) was added TBTU (170 mg, 0.52 mmol) and DIPEA (163 μl, 0.94 mmol). The mixture was stirred at RT for 30 minutes. tert-Butyl 3-aminopyrrolidine-1-carboxylate (98 μl, 0.56 mmol) was added and the reaction mixture was stirred at RT for another 2 hours. The mixture was diluted with DCM (10 mL), washed with water (2×20 mL) and brine (10 mL). The organic layer was dried (MgSO₄) and concentrated under reduced pressure. The residue was purified by column chromatography (0-1% MeOH in DCM) to afford the product as a yellow solid (220 mg, 78% yield). ESMS: m/z 594 [M+H]⁺.

### Stage 2—4-{[(7R)-8-Cyclopentyl-7-ethyl-5-methyl-6-oxo-5,6,7,8-tetrahydropteridin-2-yl]amino}-3-methoxy-N-pyrrolidin-3-ylbenzamide

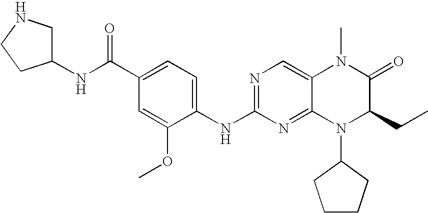

tert-Butyl 3-[(4-{[(7R)-8-cyclopentyl-7-ethyl-5-methyl-6-oxo-5,6,7,8-tetrahydro pteridin-2-yl]amino}-3-methoxybenzoyl)amino]pyrrolidine-1-carboxylate (22 mg, 0.36 mmol) was dissolved in 4M HCl dioxane (6 ml) and stirred at RT for 1 hour. The reaction was concentrated under reduced pressure to afford the product as a white solid (120 mg, 68% yield). ESMS: m/z 494 [M+H]⁺.

### Stage 3—Cyclopentyl (2S)-2-[(tert-butoxycarbonyl)amino]-4-{3-[(4-{[(7R)-8-cyclopentyl-7-ethyl-5-methyl-6-oxo-5,6,7,8-tetrahydropteridin-2-yl]amino}-3-methoxybenzoyl)amino]pyrrolidin-1-yl}butanoate

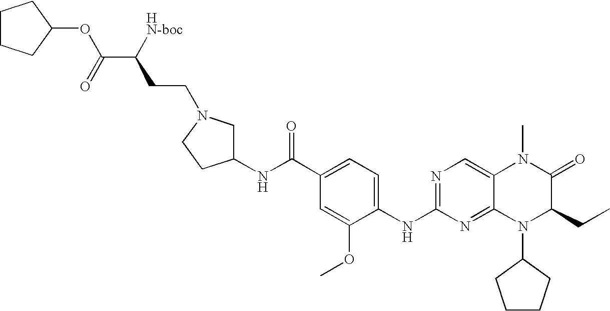

To a stirred solution of 4-{[(7R)-8-cyclopentyl-7-ethyl-5-methyl-6-oxo-5,6,7,8-tetrahydro pteridin-2-yl]amino}-3-methoxy-N-pyrrolidin-3-ylbenzamide (120 mg, 0.25 mmol) in DMF (5 mL) was added K₂CO₃ (140 mg, 1.0 mmol), NaI (75 μl, 1, 0.5 mmol) and (S)-cyclopentyl (2S)-4-bromo-2-[(tert-butoxycarbonyl)amino]butanoate [intermediate 3A] (130 mg, 0.37 mmol). The reaction mixture was stirred at 80° C. overnight and then diluted with EtOAc (10 mL). The mixture was washed with water (2×10 mL) and brine (10 mL). The organic layer was dried (MgSO₄) and concentrated under reduced pressure. The residue was purified by column chromatography (0-2% MeOH/DCM) to afford the product as a pale yellow solid (140 mg, 71% yield). ESMS: m/z 522 [M+H]⁺.

### Stage 4—Cyclopentyl (2S)-2-amino-4-{3-[(4-{[(7R)-8-cyclopentyl-7-ethyl-5-methyl-6-oxo-5,6,7,8-tetrahydropteridin-2-yl]amino}-3-methoxybenzoyl)amino]pyrrolidin-1-yl}butanoate

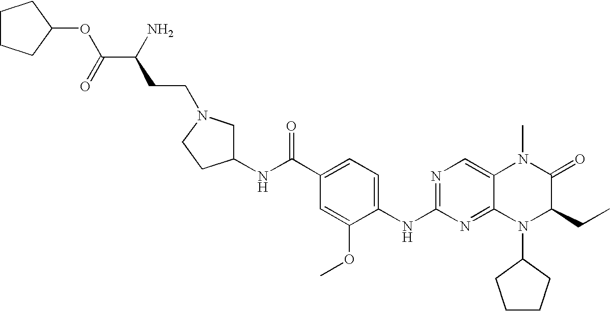

Cyclopentyl (2S)-2-[(tert-butoxycarbonyl)amino]-4-{3-[(4-{[(7R)-8-cyclopentyl-7-ethyl-5-methyl-6-oxo-5,6,7,8-tetrahydropteridin-2-yl]amino}-3-methoxybenzoyl)amino]pyrrolidin-1-yl}butanoate (140 mg, 0.18 mmol) was dissolved in 4M HCl/dioxane (5 mL) and stirred at RT for 2 hours. The reaction was concentrated under reduced pressure. The residue was triturated with Et₂O, filtered and dried under reduced pressure to afford the title example as a white solid (60 mg, 50% yield). ESMS: m/z 663 [M+H]⁺. ¹H NMR (300 MHz, CDCl₃) δ: 8.56 (1H, d, J=8.5 Hz), 7.69 (1H, s), 7.61 (1H, s), 7.51 (1H, d, J=1.7 Hz), 7.44 (1H, d, J=8.7 Hz), 6.66-6.72 (1H, m), 5.09-5.16 (1H, m), 4.47-4.69 (2H, m), 4.23 (1H, dd, J=7.9, 3.8 Hz), 3.99 (3H, s), 3.48 (1H, t, J=6.2 Hz), 3.34 (3H, s), 2.95-3.05 (1H, m), 2.83 (1H, d, J=10.0 Hz), 2.46-2.71 (3H, m), 2.11-2.45 (3H, m), 1.47-2.05 (22H, m), 0.89 (3H, t, J=7.5 Hz).

The example in the following table was prepared by methods analogous to the method described above (Scheme 23) using the appropriate intermediates.

| | Stage 3 | | |
|---|---|---|---|
| | Intermediate | | |
| Example | used | Name | ESMS |
| 17 | 4A | Cyclopentyl (2R)-2-amino-4-{3-[(4-{[(7R)-8- | m/z |
| | | cyclopentyl-7-ethyl-5-methyl-6-oxo-5,6,7,8- | 663 [M + H]⁺ |
| | | tetrahydropteridin-2-yl]amino}-3- | |
| | | methoxybenzoyl)amino]pyrrolidin-1-yl}butanoate | |

### Example 18

### Cyclopentyl (2S)-2-amino-4-{6-[(4-{[(7R)-8-cyclopentyl-7-ethyl-5-methyl-6-oxo-5,6,7,8-tetrahydropteridin-2-yl]amino}-3-methoxybenzoyl)amino]-3-azabicyclo[3.1.01]hex-3-yl}butanoate

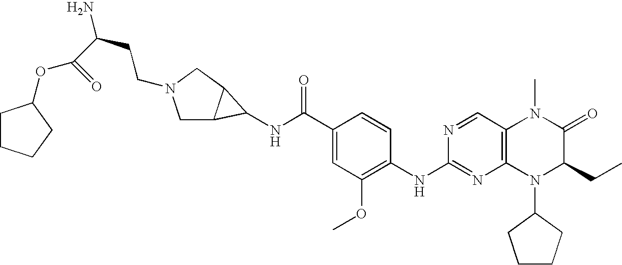

The titled example was prepared according to the procedure outlined below (Scheme 24):

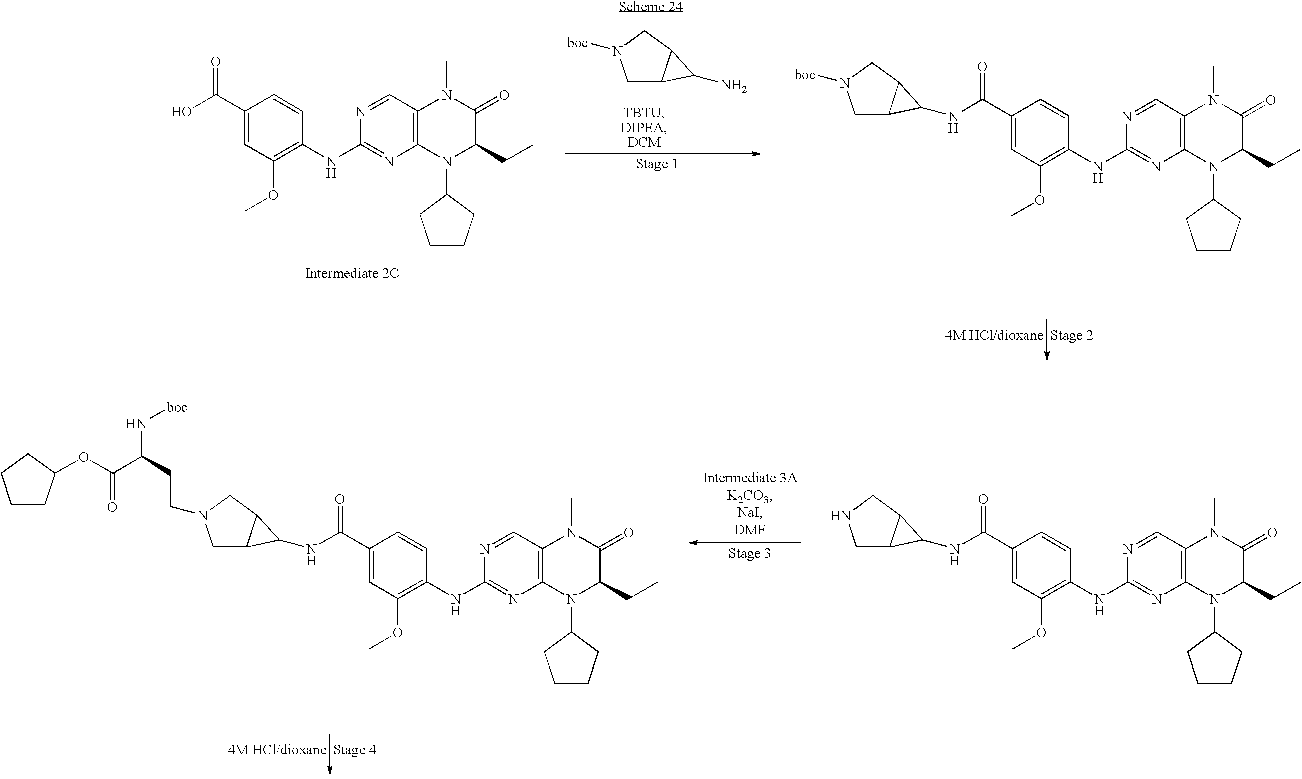

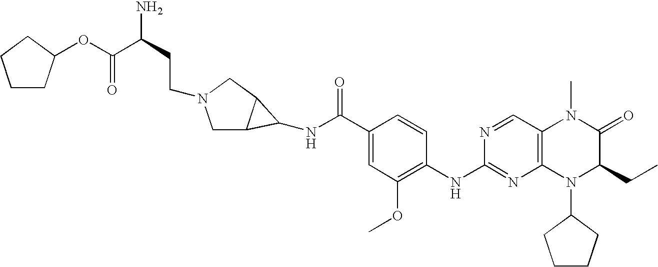

### Stage 1—tert-butyl 6-[(4-{[(7R)-8-cyclopentyl-7-ethyl-5-methyl-6-oxo-5,6,7,8-tetrahydro pteridin-2-yl]amino}-3-methoxybenzoyl)amino]-3-azabicyclo[3.1.0]hexane-3-carboxylate

To a stirred solution of 4-{[(7R)-8-cyclopentyl-7-ethyl-5-methyl-6-oxo-5,6,7,8-tetrahydropteridin-2-yl]amino}-3-methoxybenzoic acid [Intermediate 2C] (200 mg, 0.47 mmol) in DCM (10 mL) was added DIPEA (0.16 mL, 0.94 mmol) and TBTU (167 mg, 0.52 mmol). The reaction stirred at RT for 30 minutes before addition of tert-butyl 6-amino-3-azabicyclo[3.1.0]hexane-3-carboxylate [WO2006123121] (111 mg, 0.56 mmol). The reaction was stirred for a further 30 minutes and then the mixture was diluted with DCM (15 mL) and washed with water (2×5 mL). The organic layer was dried (MgSO₄) and concentrated under reduced pressure. The resulting solid was triturated with Et₂O to afford the product as a white solid (230 mg, 81% yield). ESMS: m/z 606 [M+H]⁺.

### Stage 2—N-3-azabicyclo[3.1.0]hex-6-yl-4-{[(7R)-8-cyclopentyl-7-ethyl-5-methyl-6-oxo-5,6,7,8-tetrahydropteridin-2-yl]amino}-3-methoxybenzamide

tert-Butyl 6-[(4-{[(7R)-8-cyclopentyl-7-ethyl-5-methyl-6-oxo-5,6,7,8-tetrahydro pteridin-2-yl]amino}-3-methoxybenzoyl)amino]-3-azabicyclo[3.1.0]hexane-3-carboxylate (230 mg, 0.38 mmol) was suspended in 4M HCl/dioxane (5 mL) and the reaction mixture was stirred at RT for 1.5 hours and concentrated under reduced pressure. The residue was triturated with Et₂O and then partitioned between DCM (5 mL) and sat Na₂CO₃ (5 mL). The organic layer washed with sat Na₂CO₃, dried (MgSO₄) and concentrated under reduced pressure to afford the product as a white solid (152 mg, 80% yield). ESMS: m/z 506 [M+H]⁺.

### Stage 3—Cyclopentyl (2S)-2-[(tert-butoxycarbonyl)amino]-4-{6-[(4-{[(7R)-8-cyclopentyl-7-ethyl-5-methyl-6-oxo-5,6,7,8-tetrahydropteridin-2-yl]amino}-3-methoxybenzoyl)amino]-3-azabicyclo[3.1.0]hex-3-yl}butanoate

To a stirred solution of N-3-azabicyclo[3.1.0]hex-6-yl-4-{[(7R)-8-cyclopentyl-7-ethyl-5-methyl-6-oxo-5,6,7,8-tetrahydropteridin-2-yl]amino}-3-methoxybenzamide (152 mg, 0.30 mmol) in DMF (3 mL) was added cyclopentyl (2S)-4-bromo-2-[(tert-butoxycarbonyl)amino]butanoate [Intermediate 3A] (157 mg, 0.45 mmol), K₂CO₃ (166 mg, 1.20 mmol) and NaI (90 mg, 0.60 mmol). The mixture was heated at 80° C. for 24 hours. The reaction mixture was concentrated under reduced pressure, the resulting residue was dissolved in EtOAc (10 mL) and washed with brine (10 mL). The organic layer was dried (MgSO₄) and concentrated under reduced pressure to afford the title product as a brown solid (228 mg, 98% yield). ESMS: m/z 775 [M+H]⁺.

### Stage 4—Cyclopentyl (2S)-2-amino-4-{6-[(4-{[(7R)-8-cyclopentyl-7-ethyl-5-methyl-6-oxo-5,6,7,8-tetrahydropteridin-2-yl]amino}-3-methoxybenzoyl)amino]-3-azabicyclo[3.1.0]hex-3-yl}butanoate

Cyclopentyl (2S)-2-[(tert-butoxycarbonyl)amino]-4-{6-[(4-{[(7R)-8-cyclopentyl-7-ethyl-5-methyl-6-oxo-5,6,7,8-tetrahydropteridin-2-yl]amino}-3-methoxybenzoyl)amino]-3-aza bicyclo[3.1.0]hex-3-yl}butanoate (228 mg, 0.29 mmol) was suspended in 4M HCl/dioxane (5 mL) and the reaction mixture was stirred at RT for 1.5 hours and concentrated under reduced pressure. The residue was purified using preparative HPLC and then the product concentrated by freeze drying for 60 hours. The resulting solid was dissolved in DCM (5 mL) and Na₂CO₃ (5 mL) and stirred for 20 minutes. The organic layer was separated, dried (MgSO₄) and concentrated under reduced pressure to afford the title example as a clear oil (23 mg, 12% yield). ESMS: m/z 675 [M+H]⁺. ¹H NMR (300 MHz, CDCl₃) δ: 8.47 (1H, d, J=8.5 Hz), 7.43-7.65 (3H, m), 7.25 (1H, d, J=6.6 Hz), 5.23 (2H, s), 5.16 (1H, t, J=5.9 Hz), 4.34-4.49 (1H, m), 4.15 (1H, dd, J=7.9, 3.8 Hz), 3.92 (2H, s), 3.55 (1H, dd, J=8.4, 3.9 Hz), 3.22-3.31 (4H, m), 3.18 (1H, d, J=9.0 Hz), 2.92 (1H, br. s), 2.57 (2H, t, J=8.3 Hz), 2.34-2.44 (2H, m), 1.38-2.15 (20H, m) and 0.81 (3H, t, J=7.4 Hz).

### Example 19

### Cyclopentyl 5-{4-[(4-{[(7R)-8-cyclopentyl-7-ethyl-5-methyl-6-oxo-5,6,7,8-tetrahydro-pteridin-2-yl]amino}-3-methoxybenzoyl)amino]phenyl}-L-norvalinate

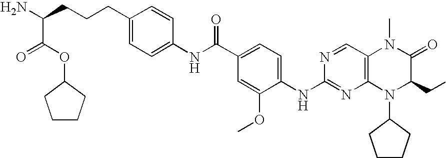

The title compound was prepared by the following methodology (Scheme 25):

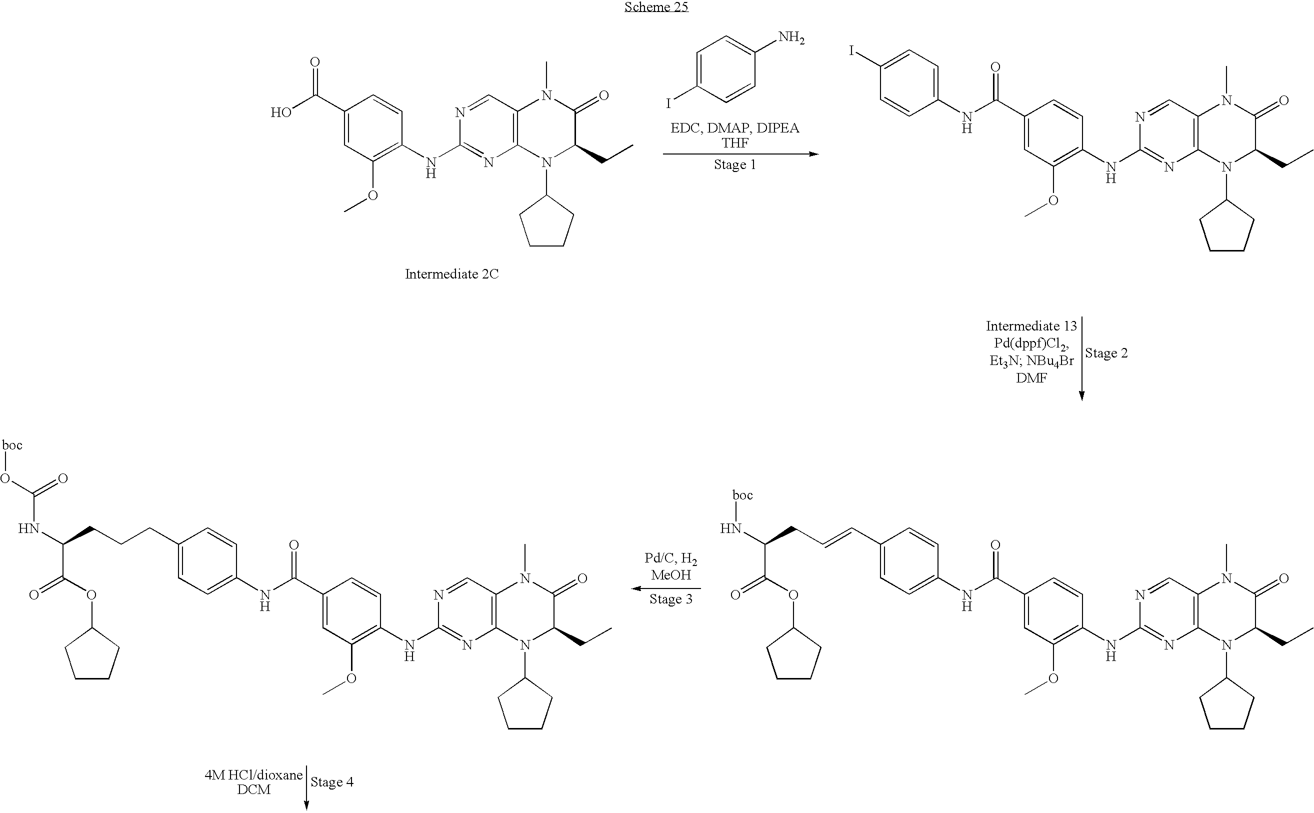

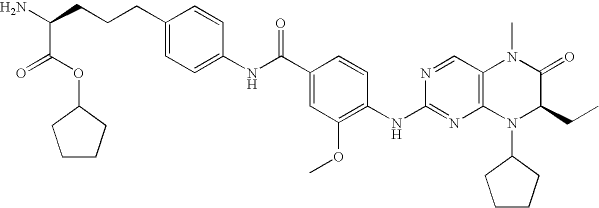

### Stage 1—4-{[(7R)-8-cyclopentyl-7-ethyl-5-methyl-6-oxo-5,6,7,8-tetrahydropteridin-2-yl]amino}-N-(4-iodophenyl)-3-methoxybenzamide

To a stirred solution of 4-{[(7R)-8-cyclopentyl-7-ethyl-5-methyl-6-oxo-5,6,7,8-tetrahydro pteridin-2-yl]amino}-3-methoxybenzoic acid [Intermediate 2C] (200 mg, 0.47 mmol) in THF (4 mL) was added 4-iodoaniline (154 mg, 0.71 mmol), DMAP (6 mg, 0.05 mmol), DIPEA (0.25 mL, 1.41 mmol) and EDC (99 mg, 0.52 mmol). The reaction mixture was stirred at RT overnight, washed with water (10 mL), dried (MgSO₄), and concentrated under reduced pressure. Purification by column chromatography (40-50% EtOAc/heptane) afforded the product (81.9 mg, 28% yield). ESMS m/z: 627 [M+H]⁺.

### Stage 2—Cyclopentyl (2S,4E)-2-[(tert-butoxycarbonyl)amino]-5-{4-[(4-{[(7R)-8-cyclopentyl-7-ethyl-5-methyl-6-oxo-5,6,7,8-tetrahydropteridin-2-yl]amino}-3-methoxy benzoyl)amino]phenyl}pent-4-enoate

To a stirred solution of 4-{[(7R)-8-cyclopentyl-7-ethyl-5-methyl-6-oxo-5,6,7,8-tetrahydropteridin-2-yl]amino}-N-(4-iodophenyl)-3-methoxybenzamide (81.9 mg, 0.13 mmol) in DMF (3 mL) was added cyclopentyl (2S)-2-[(tert-butoxycarbonyl)amino]pent-4-enoate [intermediate 13] (56 mg, 0.20 mmol), Pd(dppf)Cl₂ (11 mg, 0.01 mmol), Et₃N (40 μl, 0.29 mmol) and NBu₄Br (42 mg, 0.13 mmol). The reaction mixture was heated at 120° C. for 1 h in the microwave and concentrated under reduced pressure. The crude residue was loaded on silica and purified by column chromatography (40% EtOAc/heptane) to give the product (50 mg, 30% yield). ESMS m/z: NI.

### Stage 3—Cyclopentyl N-{[(tert-butoxycarbonyl)oxy]carbonyl}-5-{4-[(4-{[(7R)-8-cyclopentyl-7-ethyl-5-methyl-6-oxo-5,6,7,8-tetrahydropteridin-2-yl]amino}-3-methoxybenzoyl)amino]phenyl}-L-norvalinate

Cyclopentyl (2S,4E)-2[(tert-butoxycarbonyl)amino]-5-{4-[(4-{[(7R)-8-cyclopentyl-7-ethyl-5-methyl-6-oxo-5,6,7,8-tetrahydropteridin-2-yl]amino}-3-methoxybenzoyl)amino]phenyl}pent-4-enoate (50 mg, 0.06 mmol) in MeOH (5 mL) was passed through an H-Cube™ continuous hydrogenator (Thales Nanotechnology, HC-2, SS). The reaction was performed using a 30 mm CatCart™ (10% Pd/C) in full H₂ mode. A flow rate of 1 mL/min was maintained for 30 min, with a temperature of 25° C. and H₂ pressure of 1 bar. The solution was then evaporated to dryness to afford the product (50 mg, 100% yield). ESMS m/z: 784 [M+H]⁺.

### Stage 4—Cyclopentyl 5-{4-[(4-{[(7R)-8-cyclopentyl-7-ethyl-5-methyl-6-oxo-5,6,7,8-tetrahydropteridin-2-yl]amino}-3-methoxybenzoyl)amino]phenyl}-L-norvalinate

To a solution of cyclopentyl N-{[(tert-butoxycarbonyl)oxy]carbonyl}-5-{4-[(4-{[(7R)-8-cyclopentyl-7-ethyl-5-methyl-6-oxo-5,6,7,8-tetrahydropteridin-2-yl]amino}-3-methoxybenzoyl)amino]phenyl}-L-norvalinate (25 mg, 0.03 mmol) in DCM (1 mL) was added 4M HCl/dioxane (30 μl, 0.12 mmol). The reaction mixture was stirred at RT for 1 hour and evaporated under reduced pressure. Purification by preparative HPLC afforded the title example as a white solid (3 mg, 14% yield). ESMS m/z: 342 [(M+2)/2]⁺. ¹H NMR (300 MHz, MeOD) δ; ppm 8.14 (1H, d, J=8.1 Hz), 7.66-7.78 (4H, m), 7.26-7.54 (3H, m), 5.37 (1H, dd, J=4.0, 1.9 Hz), 4.53 (1H, dd, J=6.8, 3.4 Hz), 4.45 (1H, t, J=8.2 Hz), 4.09 (2H, s), 3.38 (3H, s), 2.73-2.95 (2H, m), 1.90-2.21 (10H, m), 1.62-1.87 (12H, m) and 0.94 (3H, t, J=7.5 Hz).

### Example 20

### Cyclopentyl (2S,4E)-2-amino-5-{4-[(4-{[(7R)-8-cyclopentyl-7-ethyl-5-methyl-6-oxo-5,6,7,8-tetrahydropteridin-2-yl]amino}-3-methoxybenzoyl)amino]phenyl}pent-4-enoate

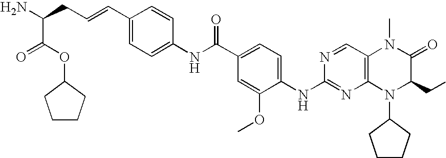

The title compound was prepared by the following methodology (Scheme 26):

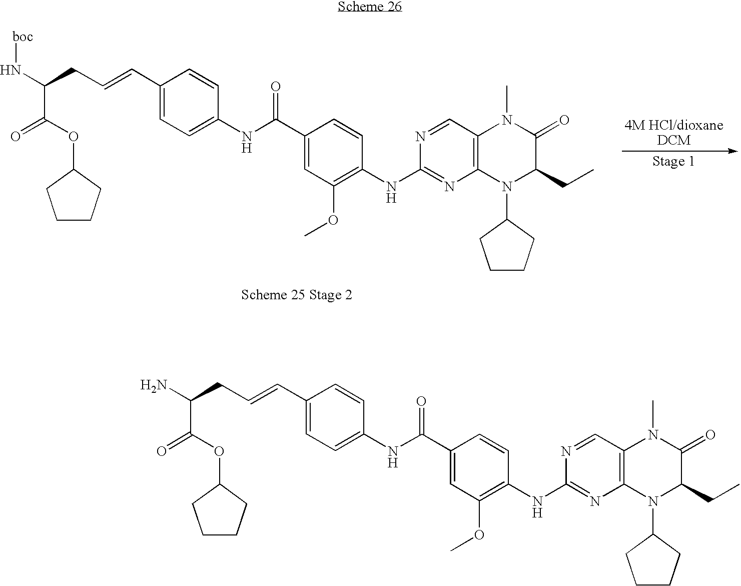

### Stage 1—Cyclopentyl (2S,4E)-2-amino-5-{4-[(4-{[(7R)-8-cyclopentyl-7-ethyl-5-methyl-6-oxo-5,6,7,8-tetrahydropteridin-2-yl]amino}-3-methoxy benzoyl)amino]phenyl}pent-4-enoate

To a stirred solution of cyclopentyl (2S,4E)-2-[(tert-butoxycarbonyl)amino]-5-{4-[(4-{[(7R)-8-cyclopentyl-7-ethyl-5-methyl-6-oxo-5,6,7,8-tetrahydropteridin-2-yl]amino}-3-methoxy benzoyl)amino]phenyl}pent-4-enoate [Scheme 25, Stage 2] (36 mg, 0.06 mmol) in DCM (2 mL) was added 4M HCl/dioxane (20 μl, 0.08 mmol). The reaction mixture was stirred at RT for 2 hours, concentrated under reduced pressure and redissolved in DCM (10 mL). The organic layer was washed with 1M NaHCO₃ (10 mL), dried (MgSO₄) and evaporated to dryness. Purification by preparative HPLC afforded the titled example as a yellow oil (3 mg, 10% yield). ESMS m/z: 342 [M/2]⁺. ¹H NMR (300 MHz, MeOD) δ: 7.55-7.62 (1H, m), 7.39-7.51 (4H, m), 6.61 (1H, d, J=15.6 Hz), 6.17 (1H, ddd, J=15.4, 7.6, 7.3 Hz), 5.26-5.34 (1H, m), 4.39 (1H, dd, J=6.3, 3.3 Hz), 4.32 (1H, t, J=8.8 Hz), 4.17 (1H, t, J=6.2 Hz), 3.25 (3H, s), 2.83 (2H, t, J=6.7 Hz), 1.79-2.06 (9H, m), 1.54-1.78 (9H, m) and 0.86 (3H, t, J=7.4 Hz).

### Example 21

### Cyclopentyl 5-{4-[(4-{[(7R)-8-cyclopentyl-7-ethyl-5-methyl-6-oxo-5,6,7,8-tetrahydro-pteridin-2-yl]-amino}-3-methoxybenzoyl)amino]phenyl}-4-hydroxy-L-norvalinate

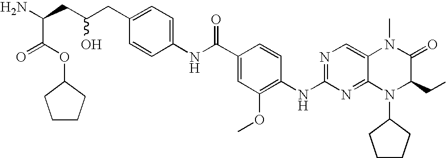

The title compound was prepared by the following methodology (Scheme 27):

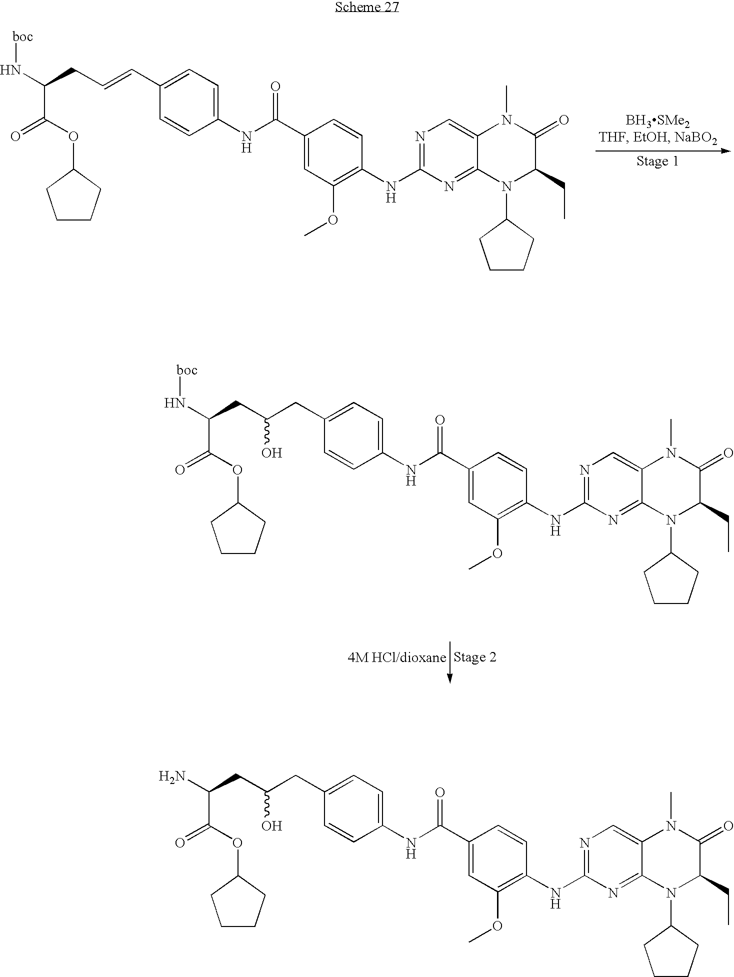

### Stage 1—Cyclopentyl N-(tert-butoxycarbonyl)-5-{4-[(4-{[(7R)-8-cyclopentyl-7-ethyl-5-methyl-6-oxo-5,6,7,8-tetrahydropteridin-2-yl]amino}-3-methoxybenzoyl)amino]phenyl}-4-hydroxy-L-norvalinate

To a solution of cyclopentyl (2S,4E)-2-[(tert-butoxycarbonyl)amino]-5-{4-[(4-{[(7R)-8-cyclopentyl-7-ethyl-5-methyl-6-oxo-5,6,7,8-tetrahydropteridin-2-yl]amino}-3-methoxy benzoyl)amino]phenyl}pent-4-enoate [Scheme 25, Stage 2] (130 mg, 0.17 mmol) in THF (2 mL) at 0° C. was added borane-dimethylsulfide complex (80 ul, 0.87 mmol). The mixture was stirred for 5 hours at 0° C. before adding ethanol (0.3 mL), water (0.27 mL) and sodium perborate tetrahydrate (133 mg, 0.87 mmol). The reaction was stirred at 0° C. for a further 3 hours and then at RT for 8 hours. The reaction mixture was concentrated, extracted in EtOAc (3×50 mL), dried (MgSO₄) and concentrated to give the product (90 mg, 75% yield). ESMS: m/z 800 [M+H]⁺.

### Stage 2—Cyclopentyl 5-{4-[(4-{[(7R)-8-cyclopentyl-7-ethyl-5-methyl-6-oxo-5,6,7,8-tetrahydropteridin-2-yl]amino}-3-methoxybenzoyl)amino]phenyl}-4-hydroxy-L-norvalinate

Procedure as in [Scheme 26, Stage 2]

ESMS: m/z 700 [M+H]⁺. ¹H NMR (300 MHz, MeOD) δ: 8.12 (1H, d, J=8.3 Hz), 7.74-7.66 (5H, m), 7.40 (2H, dd J=1.8, 8.6 Hz), 5.31 (1H, m), 4.49-4.38 (3H, m) 4.04 (3H, s), 3.33 (3H, s), 3.10-3.09 (2H, m), 2.17-1.62 (22H, m) and 0.89 (3H, t, J=7.5 Hz).

### Example 22

### Cyclopentyl (2S)-2-amino-4-{4-[(4-{[(7R)-8-cyclopentyl-7-ethyl-5-methyl-6-oxo-5,6,7,8-tetrahydropteridin-2-yl]amino}-3-methoxybenzoyl)amino]piperidin-1-yl}butanoate

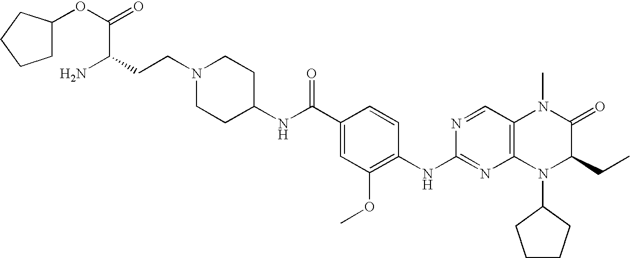

The titled example was prepared according to the general procedure outlined below (Scheme 28):

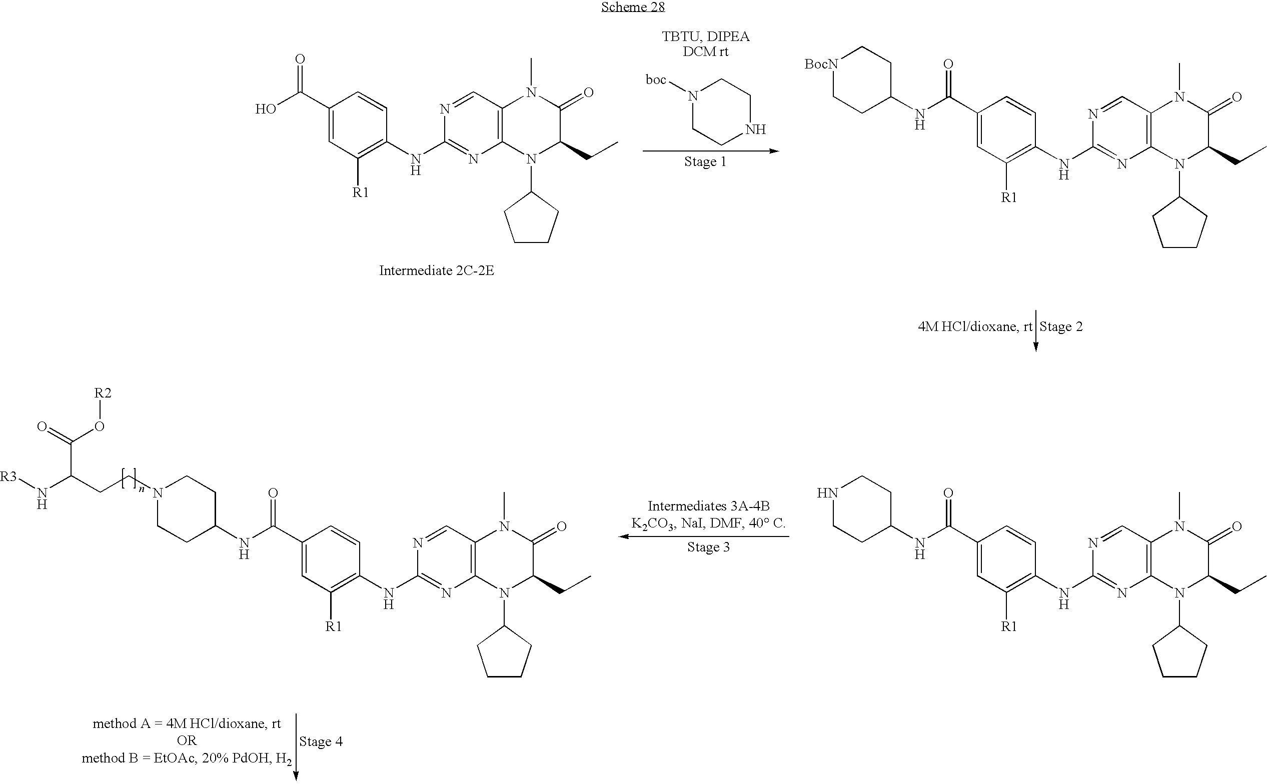

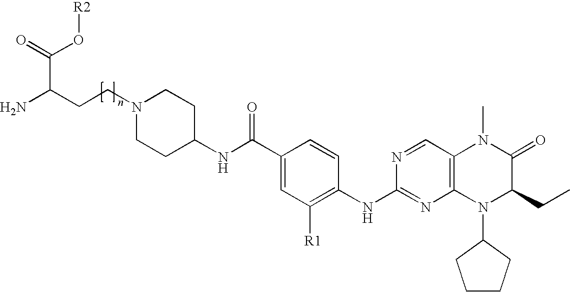

### Stage 1—tert-Butyl 4-[(4-{[(7R)-8-cyclopentyl-7-ethyl-5-methyl-6-oxo-5,6,7,8-tetrahydropteridin-2-yl]amino}-3-methoxybenzoyl)amino]piperidine-1-carboxylate

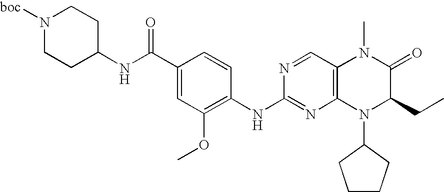

To a suspension of 4-{[(7R)-8-Cyclopentyl-7-ethyl-5-methyl-6-oxo-5,6,7,8-tetrahydro pteridin-2-yl]amino}-3-methoxybenzoic acid [Intermediate 2C] (500 mg, 1.18 mmol) in DCM (20 mL) was added TBTU (415 mg, 1.29 mmol) and DIPEA (0.41 mL, 2.35 mmol). The reaction mixture was stirred at RT for 30 minutes and then tert-butyl 4-aminopiperidine-1-carboxylate (282 mg, 1.41 mmol) was added. The reaction mixture was stirred at RT for another 30 minutes and then diluted with DCM (30 mL). The solution was washed with water (2×30 mL), dried (MgSO₄) and concentrated under reduced pressure to leave a thick brown oil. Trituration with Et₂O/heptane (1:3) afforded the product as a beige solid (528 mg, 74% yield). ESMS: m/z 608 [M+H]⁺.

### Stage 2—4-{[7R)-8-cyclopentyl-7-ethyl-5-methyl-6-oxo-5,6,7,8-tetrahydropteridin-2-yl]amino}-3-methoxy-N-piperidin-4-ylbenzamide

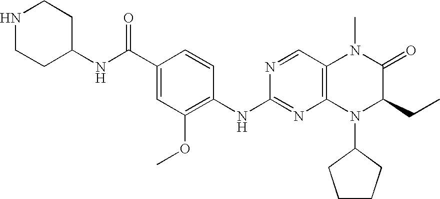

tert-Butyl 4-[(4-{[(7R)-8-cyclopentyl-7-ethyl-5-methyl-6-oxo-5,6,7,8-tetrahydropteridin-2-yl]amino}-3-methoxybenzoyl)amino]piperidine-1-carboxylate (528 mg, 0.87 mmol) was suspended in a solution of 4M HCl/dioxane (10 mL). The reaction mixture was stirred at RT for 1 hour and concentrated under reduced pressure. The residue was triturated with Et₂O and then partitioned between DCM (100 mL) and sat. Na₂CO₃ (50 mL). The organic layer was separated, washed with sat. Na₂CO₃ (50 mL), dried (MgSO₄) and concentrated under reduced pressure to afford the product as a thick yellow oil, which solidified on standing (407 mg, 92% yield). ESMS: m/z 508 [M+H]⁺.

### Stage 3—Cyclopentyl (2S)-2-[(tert-butoxycarbonyl)amino]-4-{4-[(4-{[(7R)-8-cyclopentyl-7-ethyl-5-methyl-6-oxo-5,6,7,8-tetrahydropteridin-2-yl]amino}-3-methoxybenzoyl)amino]piperidin-1-yl}butanoate

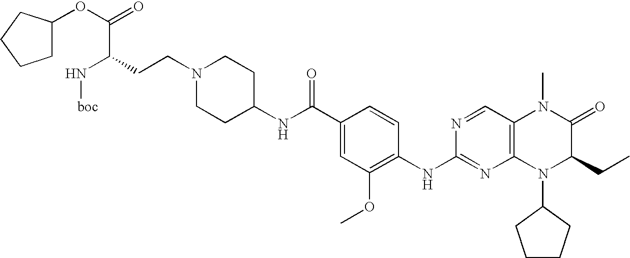

To a solution of 4-{[(7R)-8-cyclopentyl-7-ethyl-5-methyl-6-oxo-5,6,7,8-tetrahydropteridin-2-yl]amino}-3-methoxy-N-piperidin-4-ylbenzamide (100 mg, 0.20 mmol) in DMF (2 mL) was added cyclopentyl (2S)-4-bromo-2-[(tert-butoxycarbonyl)amino]butanoate [Intermediate 3A] (103 mg, 0.30 mmol), K₂CO₃ (109 mg, 0.79 mmol) and NaI (59 mg, 0.40 mmol). The reaction mixture was stirred at 80° C. for 15 hours, diluted with EtOAc (20 mL), washed with water (2×20 mL), brine (20 mL) and dried (MgSO₄). The solvent was concentrated under reduced pressure to leave a yellow oil. Purification by column chromatography (5% MeOH/DCM) afforded the product as a white solid (86 mg, 56% yield). ESMS: m/z 777 [M+H]⁺.

### Stage 4 (Method A)—Cyclopentyl (2S)-2-amino-4-{4-[(4-{[(7R)-8-cyclopentyl-7-ethyl-5-methyl-6-oxo-5,6,7,8-tetrahydropteridin-2-yl]amino}-3-methoxybenzoyl)amino]piperidin-1-yl}butanoate

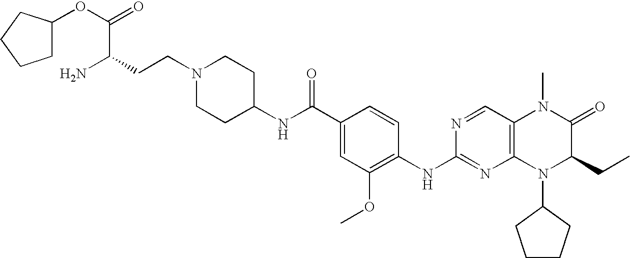

Cyclopentyl (2S)-2-[(tert-butoxycarbonyl)amino]-4-{4-[(4-{[(7R)-8-cyclopentyl-7-ethyl-5-methyl-6-oxo-5,6,7,8-tetrahydropteridin-2-yl]amino}-3-methoxybenzoyl)amino]piperidin-1-yl}butanoate (86 mg, 0.11 mmol) was suspended in a solution of 4M HCl/dioxane (5 mL). The reaction mixture was stirred at RT for 20 minutes and concentrated under reduced pressure. The residue was triturated with Et₂O and then partitioned between DCM (25 mL) and sat. Na₂CO₃ (25 mL). The organic layer was separated, dried (MgSO₄) and concentrated under reduced pressure to afford the title example as a white solid (49 mg, 65% yield). ESMS m/z 677 [M+H]⁺. ¹H NMR (300 MHz, CD₃OD) 8.49 (1H, d, J=9.0 Hz), 7.77 (1H, s), 7.50-7.47 (2H, m), 5.24-5.19 (1H, m), 4.54-4.47 (1H, m), 4.28 (1H, dd, J=3.5, 7.7 Hz), 4.01 (3H, s), 3.95-3.87 (1H, m), 3.66-3.59 (1H, m), 3.32 (3H, s), 3.01 (2H, s), 2.50 (2H, t, J=7.2 Hz), 2.19-2.10 (2H, m), 1.99-1.68 (23H, m) and 0.86 (3H, t, J=7.5 Hz).

### Example 23

### tert-butyl 5-{4-[(4-{[(7R)-8-cyclopentyl-7-ethyl-5-methyl-6-oxo-5,6,7,8-tetrahydropteridin-2-yl]amino}-3-methoxybenzoyl)amino]piperidin-1-yl}-L-norvalinate

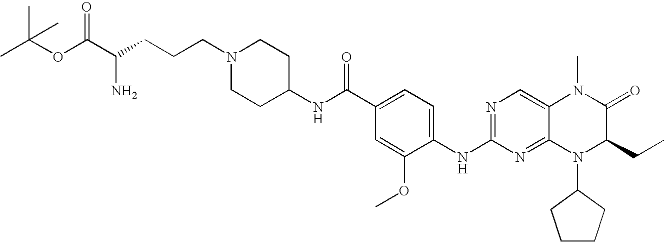

The titled example was prepared according to the general procedure and methodology outlined above (Scheme 28)

Stages 1-3 As Scheme 28 in using intermediates 2C (stage 1) and 3D (stage 3).

The stage 4 deprotection step was carried out using method B as outlined below.

### Stage 4 (Method B)—tert-butyl 5-{4-[(4-{[(7R)-8-cyclopentyl-7-ethyl-5-methyl-6-oxo-5,6,7,8-tetrahydropteridin-2-yl]amino}-3-methoxybenzoyl)amino]piperidin-1-yl}-L-norvalinate

To a solution of the stage 3 product; tert-butyl N-[(benzyloxy)carbonyl]-5-{4-[(4-{[(7R)-8-cyclopentyl-7-ethyl-5-methyl-6-oxo-5,6,7,8-tetrahydropteridin-2-yl]amino}-3-methoxybenzoyl)amino]piperidin-1-yl}-L-norvalinate (290 mg, 0.36 mmol) in EtOAc (6 mL) was added palladium hydroxide (60 mg, 20% wt/wt.). The system was evacuated and put under a hydrogen atmosphere (using a 3-way tap apparatus and hydrogen-filled balloon), this was repeated twice and the mixture was allowed to stir for 90 hour at RT under a hydrogen atmosphere. The system was evacuated of hydrogen and the palladium residues filtered over Celite®. The Celite® was washed thoroughly with EtOAc and the combined filtrates evaporated under reduced pressure. The residue was purified by column chromatography (100% EtOAc to remove impurities followed by 5-10% MeOH/DCM to elute product) to afford the title example as a white solid (37 mg, 15% yield). ESMS: m/z 679 [M+H]⁺. ¹H NMR (300 MHz, CDCl₃) δ: 8.53 (1H, d, J=8.5 Hz), 7.67 (1H, s), 7.58 (1H, s), 7.47 (1H, d, J=1.5 Hz), 7.34 (1H, dd, J=8.5, 1.5 Hz), 6.45 (1H, d, J=7.5 Hz), 4.50 (1H, t, J=7.7 Hz), 4.21 (1H, dd, J=7.8, 3.7 Hz), 4.00-4.10 (1H, m), 3.97 (3H, s), 3.31-3.44 (1H, m), 3.32 (3H, s), 2.95 (2H, d, J=8.9 Hz), 2.40 (2H, t, J=6.7 Hz), 1.59-2.21 (20H, m), 1.45 (9H, s) and 0.87 (3H, t, J=7.4 Hz).

The examples in the following table were prepared by methods analogous to the method described above (Scheme 28) using the appropriate intermediates.

| | Intermediates | | | | |
|---|---|---|---|---|---|
| | used | | Stage 4 | | |
| Example | Stage 1 | Stage 3 | method | Name | ESMS |
| 24 | 2C | 3C | A | Cyclopentyl 5-{4-[(4-{[(7R)-8-cyclo pentyl-7-ethyl- | m/z |
| | | | | 5-methyl-6-oxo-5,6,7,8-tetrahydro pteridin-2- | 691 |
| | | | | yl]amino}-3-methoxybenzoyl) amino]piperidin-1- | [M + H]⁺ |
| | | | | yl}-L-norvalinate | |
| 25 | 2C | 3B | B | t-Butyl (2S)-2-amino-4-{4-[(4-{[(7R)-8- | m/z |
| | | | | cyclopentyl-7-ethyl-5-methyl-6-oxo-5,6,7,8- | 665 |
| | | | | tetrahydropteridin-2-yl]amino}-3-methoxy | [M + H]⁺ |
| | | | | benzoyl) amino] piperidin-1-yl}butanoate | |
| 26 | 2D | 3B | B | t-Butyl (2S)-2-amino-4-{4-[(4-{[(7R)-8- | m/z |
| | | | | cyclopentyl-7-ethyl-5-methyl-6-oxo-5,6,7,8-tetra | 649 |
| | | | | hydropteridin-2-yl]amino}-3-methylbenzoyl) | [M + H]⁺ |
| | | | | amino] piperidin-1-yl}butanoate | |
| 27 | 2C | 4B | A | Cyclopentyl 5-{4-[(4-{[(7R)-8-cyclopentyl-7-ethyl- | m/z |
| | | | | 5-methyl-6-oxo-5,6,7,8-tetrahydropteridin-2- | 691 |
| | | | | yl]amino}-3-methoxybenzoyl)amino]piperidin-1- | [M + H]⁺ |
| | | | | yl}-D-norvalinate | |
| 28 | 2D | 3A | A | Cyclopentyl (2S)-2-amino-4-(4-[(4-{[(7R)-8- | m/z |
| | | | | cyclopentyl-7-ethyl-5-methyl-6-oxo-5,6,7,8-tetra | 661 |
| | | | | hydropteridin-2-yl]amino}-3-methylbenzoyl) | [M + H]⁺ |
| | | | | amino] piperidin-1-yl}butanoate | |
| 29 | 2E | 3B | B | t-Butyl (2S)-2-amino-4-{4-{(4-{[(7R)-8- | m/z |
| | | | | cyclopentyl-7-ethyl-5-methyl-6-oxo-5,6,7,8- | 653 |
| | | | | tetrahydropteridin-2-yl]amino}-3-fluorobenzoyl) | [M + H]⁺ |
| | | | | amino] piperidin-1-yl}butanoate | |
| 30 | 2E | 3A | A | Cyclopentyl (2S)-2-amino-4-{4-[(4-{[(7R)-8- | m/z |
| | | | | cyclopentyl-7-ethyl-5-methyl-6-oxo-5,6,7,8-tetra | 665 |
| | | | | hydropteridin-2-yl]amino}-3-fluorobenzoyl)amino] | [M + H]⁺ |
| | | | | piperidin-1-yl}butanoate | |
| 31 | 2C | 4A | A | Cyclopentyl (2R)-2-amino-4-{4-[(4-{((7R)-8- | m/z |
| | | | | cyclopentyl-7-ethyl-5-methyl-6-oxo-5,6,7,8-tetra | 677 |
| | | | | hydropteridin-2-yl]amino}-3-methoxybenzoyl) | [M + H]⁺ |
| | | | | amino] piperidin-1-yl}butanoate | |

### Example 32

### (1R,2S,5R)-2-isopropyl-5-methylcyclohexyl (2S)-2-amino-4-{4-[4-{[(7R)-8-cyclopentyl-7-ethyl-5-methyl-6-oxo-5,6,7,8-tetrahydropteridin-2-yl]amino}-3-methoxybenzoyl) amino]piperidin-1-yl}butanoate

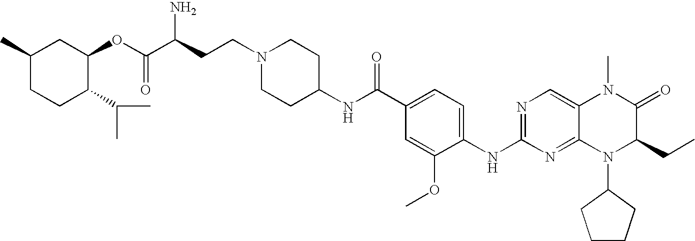

The title compound was prepared by the following methodology (Scheme 29):

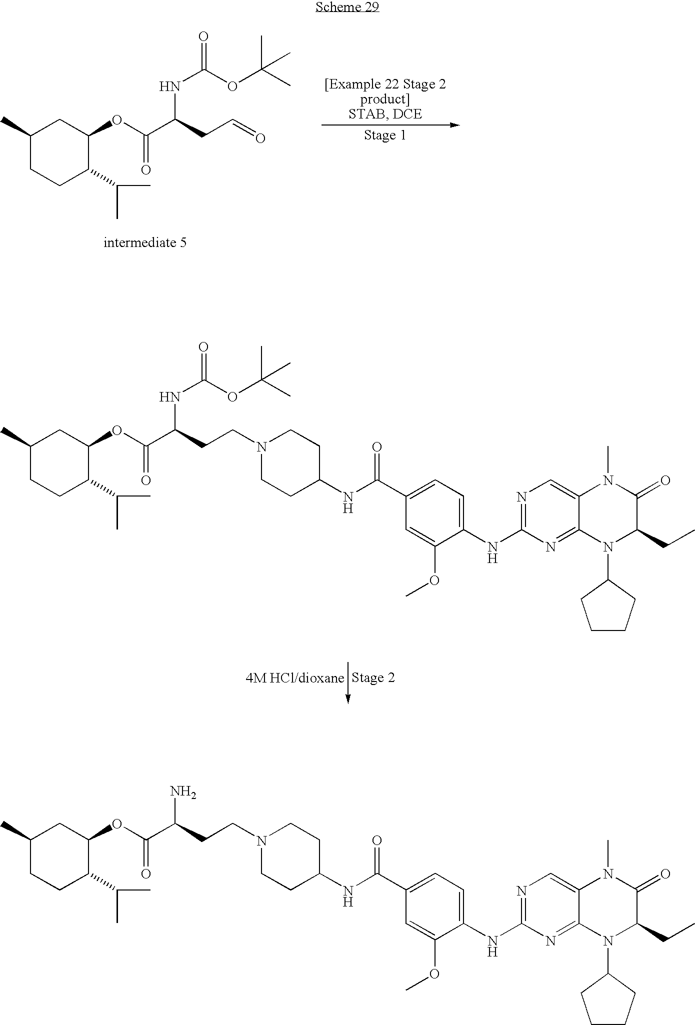

### Stage 1—(1R,2S,5R)-2-isopropyl-5-methylcyclohexyl (2S)-2-[(tert-butoxycarbonyl)amino]-4-{4-[(4-{[(7R)-8-cyclopentyl-7-ethyl-5-methyl-6-oxo-5,6,7,8-tetrahydropteridin-2-yl]amino}-3-methoxybenzoyl)amino]piperidin-1-yl}butanoate

To a solution of (1R,2S,5R)-2-isopropyl-5-methylcyclohexyl (2S)-2-[(tert-butoxycarbonyl)amino]-4-oxobutanoate [Intermediate 5] (140 mg, 0.39 mmol) in DCE (15 mL) was added 4-{[(7R)-8-cyclopentyl-7-ethyl-5-methyl-6-oxo-5,6,7,8-tetrahydropteridin-2-yl]amino}-3-methoxy-N-piperidin-4-ylbenzamide [Example 22, Stage 2] (108 mg, 0.30 mmol). The solution was stirred for 30 min before addition of sodium triacetoxy-borohydride (193 mg, 0.91 mmol). The reaction stirred for a further 18 hours at RT. Sat NaHCO₃ (10 mL) was added and the reaction stirred for 20 minutes. DCM (10 mL) was added and the organic layer separated. The aqueous layer was extracted with DCM (2×10 mL). The combined organic layers were dried (MgSO₄) and concentrated under reduced pressure. Purification by flash column chromatography (2% MeOH/DCM) afforded the product as a clear oil (68 mg, 24% yield). ESMS m/z 847 [M+H]⁺.

### Stage 2—(1R,2S,5R)-2-isopropyl-5-methylcyclohexyl (2S)-2-amino-4-{4-[(4-{[(7R)-8-cyclopentyl-7-ethyl-5-methyl-6-oxo-5,6,7,8-tetrahydropteridin-2-yl]amino}-3-methoxybenzoyl)amino]piperidin-1-yl}butanoate

To a solution of (1R,2S,5R)-2-isopropyl-5-methylcyclohexyl (2S)-2-[(tert-butoxycarbonyl)amino]-4-{4-[(4-{[(7R)-8-cyclopentyl-7-ethyl-5-methyl-6-oxo-5,6,7,8-tetrahydropteridin-2-yl]amino}-3-methoxybenzoyl)amino]piperidin-1-yl}butanoate (11 mg, 0.01 mmol) in DCM (1 mL) was added 4M HCl/dioxane (1 mL). The solution was stirred at RT for 3 hours. The mixture was then concentrated under reduced pressure to give a the title example as a white solid (6.1 mg, 63% yield). ESMS m/z 747 [M+H]⁺. ¹H NMR (300 MHz, MeOD) δ 7.90 (1H, d, J=8.3 Hz), 7.68-7.55 (3H, m), 4.56-4.47 (1H, m), 4.45-4.07 (4H, m), 4.01 (3H, s), 3.75 (2H, m), 3.67 (2H, s), 2.77 (1H, m), 2.64-1.06 (38H, m) and 082 (3H, d, J=7.0 Hz).

### Example 33

### 4-[(4-{[7R)-8-cyclopentyl-7-ethyl-5-methyl-6-oxo-5,6,7,8-tetrahydropteridin-2-yl]amino}-3-methoxybenzoyl)amino]-L-phenylalanine

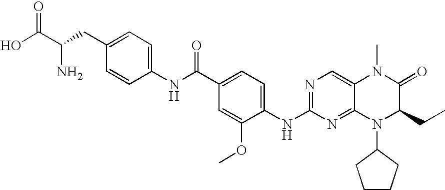

The title compound was prepared by the following methodology (Scheme 30):

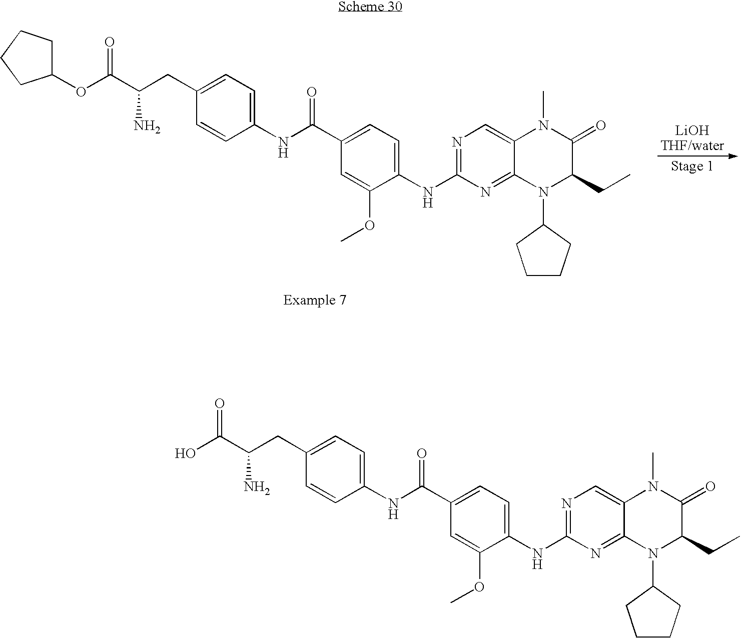

### Stage 1—4-[(4-{[(7R)-8-cyclopentyl-7-ethyl-5-methyl-6-oxo-5,6,7,8-tetrahydropteridin-2-yl]amino}-3-methoxybenzoyl)amino]-L-phenylalanine

To cyclopentyl 4-[(4-{[(7R)-8-cyclopentyl-7-ethyl-5-methyl-6-oxo-5,6,7,8-tetrahydro pteridin-2-yl]amino}-3-methoxybenzoyl)amino]-L-phenylalaninate [Example 7] (45 mg, 70 μmol) in THF (3 mL) was added to a solution of lithium hydroxide (8.4 mg, 0.35 mmol) in water (3 mL). The reaction mixture was stirred at RT overnight and concentrated under reduced pressure. Water (4 mL) was added and the pH was adjusted to pH=5-6 with 1M HCl. The aqueous was extracted with n-butanol (3×10 mL). The combined organic layers were washed with water (5 mL), brine (5 mL), dried (MgSO₄) and concentrated under reduced pressure. Purification by preparative HPLC afforded the title example as a white solid (37 mg, 90% yield). ESMS m/z: 588 [M+H]⁺. ¹H NMR (300 MHz, DMSO-d₆) δ: 10.17 (1H, s), 8.48-8.75 (1H, m), 8.18-8.34 (4H, m), 7.83 (1H, s), 7.73 (2H, d, J=8.7 Hz), 7.61-7.67 (2H, m), 7.23 (2H, d, J=8.5 Hz), 4.36 (1H, dd, J=6.8, 3.2 Hz), 4.14-4.30 (2H, m), 3.96 (3H, s), 3.23 (3H, s), 3.07 (2H, d, J=6.4 Hz), 1.43-2.04 (10H, m) and 0.76 (3H, t, J=7.4 Hz).

The examples in the following table were prepared by the ester hydrolysis method described above (Scheme 30).

| Ester | | Acid | |
|---|---|---|---|
| Example | | Example | |
| No. | Acid Name | No. | ESMS |
| 1 | 4-{[(7R)-8-Cyclopentyl-7-ethyl-5-methyl-6-oxo-5,6,7,8- | 34 | m/z |
| | tetrahydropteridin-2-yl]amino}-L-phenylalanine | | 439 [M + H]⁺ |
| 2 | (2S,4E)-2-Amino-5-(4-{[(7R)-8-cyclopentyl-7-ethyl-5- | 35 | m/z: |
| | methyl-6-oxo-5,6,7,8-tetrahydropteridin-2- | | 465 [M + H]⁺ |
| | yl]amino}phenyl)pent-4-enoic acid | | |
| 3 | O-(4-{[(7R)-8-Cyclopentyl-7-ethyl-5-methyl-6-oxo-5,6,7,8- | 36 | m/z |
| | tetrahydropteridin-2-yl]amino}phenyl)-L-homoserine | | 469 [M + H]⁺ |
| 4 | O-(4-{[(7R)-8-Cyclopentyl-7-ethyl-5-methyl-6-oxo-5,6,7,8- | 37 | m/z |
| | tetrahydropteridin-2-yl]amino}-3-methoxyphenyl)-L- | | 499 [M + H]⁺ |
| | homoserine | | |
| 5 | (2S)-2-Amino-4-[(4-{[(7R)-8-cyclopentyl-7-ethyl-5-methyl- | 38 | m/z |
| | 6-oxo-5,6,7,8-tetrahydropteridin-2-yl]amino}-3- | | 526 [M + H]⁺ |
| | methoxybenzoyl)amino]butananoic acid | | |
| 6 | (4S)-4-[(4-{[(7R)-8-Cyclopentyl-7-ethyl-5-methyl-6-oxo- | 39 | m/z |
| | 5,6,7,8-tetrahydropteridin-2-yl]amino}-3- | | 538 [M + H]⁺ |
| | methoxybenzoyl)amino]-L-proline | | |
| 8 | N⁶-(4-{[(7R)-8-cyclopentyl-7-ethyl-5-methyl-6-oxo- | 40 | m/z |
| | 5,6,7,8-tetrahydropteridin-2-yl]amino}-3- | | 554 [M + H]⁺ |
| | methoxybenzoyl)lysine | | |
| 9 | O-(4-{[(4-{[(7R)-8-Cyclopentyl-7-ethyl-5-methyl-6-oxo- | 41 | m/z |
| | 5,6,7,8-tetrahydropteridin-2-yl]amino}-3- | | 632 [M + H]⁺ |
| | methoxybenzoyl)amino]methyl} phenyl)-L-homoserine | | |
| 13 | 4-(4-{[(7R)-8-Cyclopentyl-7-ethyl-5-methyl-6-oxo-5,6,7,8- | 42 | m/z |
| | tetrahydropteridin-2-yl]amino}-3- | | 538 [M + H]⁺ |
| | methoxybenzoyl)piperazine-2-carboxylic acid | | |
| 14 | 4-{2-[(4-{[(7R)-8-Cyclopentyl-7-ethyl-5-methyl-6-oxo- | 43 | m/z |
| | 5,6,7,8-tetrahydro pteridin-2-yl]amino}-3- | | 581 [M + H]⁺ |
| | methoxybenzoyl) amino]ethyl}piperazine-2-carboxylic | | |
| | acid | | |
| 16 | (2S)-2-amino-4-{3-[(4-{[(7R)-8-Cyclopentyl-7-ethyl-5- | 44 | m/z |
| | methyl-6-oxo-5,6,7,8-tetrahydropteridin-2-yl]amino}-3- | | 595 [M + H]⁺ |
| | methoxybenzoyl)amino]pyrrolidin-1-yl}butanoic acid | | |
| 18 | (2S)-2-Amino-4-{6-[(4-{[(7R)-8-cyclopentyl-7-ethyl-5- | 45 | m/z |
| | methyl-6-oxo-5,6,7,8-tetrahydropteridin-2-yl]amino}-3- | | 607 [M + H]⁺ |
| | methoxybenzoyl)amino]-3-azabicyclo[3.1.0]hex-3-yl} | | |
| | butanoic acid | | |
| 24 | 5-{4-[(4-{[(7R)-8-Cyclo pentyl-7-ethyl-5-methyl-6-oxo- | 46 | m/z |
| | 5,6,7,8-tetrahydro pteridin-2-yl]amino}-3- | | 623 [M + H]⁺ |
| | methoxybenzoyl)amino]piperidin-1-yl}-L-norvaline | | |
| 27 | 5-{4-[(4-{[(7R)-8-Cyclopentyl-7-ethyl-5-methyl-6-oxo- | 47 | m/z |
| | 5,6,7,8-tetrahydropteridin-2-yl]amino}-3- | | 623 [M + H]⁺ |
| | methoxybenzoyl)amino]piperidin-1-yl}-D-norvaline | | |
| 28 | (2S)-2-Amino-4-{4-[(4-{[(7R)-8-cyclopentyl-7-ethyl-5- | 48 | m/z |
| | methyl-6-oxo-5,6,7,8-tetra hydropteridin-2-yl]amino}-3- | | 593 [M + H]⁺ |
| | methylbenzoyl)amino] piperidin-1-yl}butanoic acid | | |
| 30 | (2S)-2-Amino-4-{4-[(4-{[(7R)-8-cyclopentyl-7-ethyl-5- | 49 | m/z |
| | methyl-6-oxo-5,6,7,8-tetra hydropteridin-2-yl]amino}-3- | | 598 [M + H]⁺ |
| | fluorobenzoyl)amino] piperidin-1-yl}butanoic acid | | |
| 31 | (2R)-2-Amino-4-{4-[(4-{[(7R)-8-cyclopentyl-7-ethyl-5- | 50 | m/z |
| | methyl-6-oxo-5,6,7,8-tetra hydropteridin-2-yl]amino}-3- | | 609 [M + H]⁺ |
| | methoxybenzoyl)amino] piperidin-1-yl}butanoic acid | | |
| 7 | 4-[(4-{[(7R)-8-cyclopentyl-7-ethyl-5-methyl-6-oxo- | 51 | m/z |
| | 5,6,7,8-tetrahydropteridin-2-yl]amino}-3- | | 588 [M + H]⁺ |
| | methoxybenzoyl)amino]-L-phenylalanine | | |
| 13 | 4-(4-{[(7R)-8-cyclopentyl-7-ethyl-5-methyl-6-oxo- | 52 | m/z |
| | 5,6,7,8-tetrahydropteridin-2-yl]amino}-3- | | 539 [M + H]⁺ |
| | methoxbenzoyl)piperazine-2-carboxylic acid | | |
| 22 | (2S)-2-amino-4-{4-[(4-{[(7R)-8-cyclopentyl-7-ethyl-5- | 53 | m/z |
| | methyl-6-oxo-5,6,7,8-tetrahydropteridin-2-yl]amino}-3- | | 609 [M + H]⁺ |
| | methoxybenzoyl)amino]piperidin-1-yl}butanoic acid | | |

### Measurement of Biological Activity

### PLK1 Enzyme Assay

The ability of compounds to inhibit PLK-1 kinase activity was measured in an assay performed by Invitrogen (Paisley, UK). The Z′-LYTE™ biochemical assay employs a fluorescence-based, coupled-enzyme format and is based on the differential sensitivity of phosphorylated and non-phosphorylated peptides to proteolytic cleavage. The peptide substrate is labelled with two fluorophores—one at each end—that make up a FRET pair. In the primary reaction, the kinase transfers the gamma-phosphate of ATP to a single serine or threonine residue in a synthetic FRET-peptide. In the secondary reaction, a site-specific protease recognizes and cleaves non-phosphorylated FRET-peptides. Phosphorylation of FRET-peptides suppresses cleavage by the Development Reagent. Cleavage disrupts FRET between the donor (i.e., coumarin) and acceptor (i.e., fluorescein) fluorophores on the FRET-peptide, whereas uncleaved, phosphorylated FRET-peptides maintain FRET. A radiometric method, which calculates the ratio (the Emission Ratio) of donor emission to acceptor emission after excitation of the donor fluorophore at 400 nm, is used to quantitate reaction progress.

The final 10 μL Kinase Reaction consists of 2.8-25.3 ng PLK1, 2 μM Ser/Thr 16 Peptide substrate and ATP in 50 mM HEPES pH 7.5, 0.01% BRIJ-35, 10 mM MgCl2, 1 mM EGTA. The assay is performed at an ATP concentration at, or close to, the Km. After the 60 minute Kinase Reaction incubation at RT, 5 μL of a 1:8 dilution of Development Reagent is added. The assay plate is incubated for a further 60 minutes at RT and read on a fluorescence plate reader.

Duplicate data points are generated from a ⅓ log dilution series of a stock solution of test compound in DMSO. Nine dilutions steps are made from a top concentration of 10 μM, and a ‘no compound’ blank is included. Data is collected and analysed using XLfit software from IDBS. The dose response curve is curve fitted to model number 205 (sigmoidal dose-response model). From the curve generated, the concentration giving 50% inhibition is determined and reported.

IC50 results were allocated to one of 3 ranges as follows:

### Range A: IC50<100 nM

Range B: IC50 from 100 nM to 500 nM

### Range C: IC50>500 nM

NT=Not tested

The results obtained for compounds of the Examples herein are given in the table below.

### Cell Inhibition Assay

Cell inhibition assays were carried out using either method A or method B

### Method A

Cells were seeded in 96W tissue culture plates (1 well=30 mm²) at a density of 50 μl cells per well in 50 μL of the appropriate culture medium (see below). 24 hrs later 500 of the compound prepared in the same medium was added as 4 fold dilutions to give final concentrations in the range 0.15 nM-2500 nM (n=6 for each concentration). The plates were then incubated at 37° C., 5% CO₂ for 120 hrs. Cell proliferation was assessed using WST-1 (a metabolic indicator dye, Roche Cat no. 1 644 807) according to the manufacturers instructions. The results were calculated as percentage of vehicle response and IC50 values represent the concentration of compound that inhibited the vehicle response by 50%.

HCT-116 culture medium —Dulbeccos MEM (Sigma D6546) plus 10% heat inactivated fetal calf serum (Hyclone SH30071 Thermo Fischer Scientific) containing 2 mM Glutamine (Sigma cat no G-7513) and 50 U/ml Penicillin and Streptomycin Sulphate (Sigma Cat no P-0781).

### Method B

Cells were seeded in 96W tissue culture plates in 50 μl of the appropriate culture medium (1 well=30 mm²) at a density according to cell type [HCT-116, 750 cells/well, Hut-78 & U937, 1500 cells/well].

24 hrs later 50 μl of the compound prepared in the same medium was added, made as 12 fold dilutions to give final concentrations from 10000 nM to 0.28 pM (n=6 for each concentration).

The plates were then incubated at 37° C., 5% CO₂ for 72 hrs.

A tritiated thymidine incorporation assay was used as a measure of cell proliferation. In short, cells were incubated with 0.4 μCi/well for 4 hrs before harvesting onto filtermats. These were dried, meltilex scintillation sheets melted on, then sealed in bags and ³H emission counted on a Trilux microbeta counter.

The results are calculated as percentage of vehicle response and 1050 values represent the concentration of compound that inhibits the vehicle response by 50%.

IC50 results were allocated to one of 3 ranges as follows:

### Range A: IC50<100 nM

Range B: IC50 from 100 nM to 500 nM

### Range C: IC50>500 nM

NT=Not tested

The results obtained for compounds of the Examples herein are given in the table below.

| | | Inhibitor Activity vs |
|---|---|---|
| | Inhibitor Activity vs | HCT 116 cell line |
| Example Number | PLK1 | (method A) |
| 1 | A | B |
| 2 | A | A |
| 3 | A | B |
| 4 | A | A |
| 5 | A | A |
| 6 | A | A |
| 7 | B | A |
| 8 | A | A |
| 9 | B | A |
| 10 | A | A |
| 11 | A | B |
| 12 | A | A |
| 13 | A | B |
| 14 | A | A |
| 15 | A | A |
| 16 | A | A |
| 17 | A | A |
| 18 | A | A |
| 19 | A | A |
| 20 | A | A |
| 21 | NT | NT |
| 22 | A | A |
| 23 | A | A |
| 24 | A | A |
| 25 | A | A |
| 26 | A | A |
| 27 | A | A |
| 28 | A | A |
| 29 | A | A |
| 30 | A | A |
| 31 | A | A |
| 32 | A | NT |
| 33 | A | NT |
| 34 | A | NT |
| 35 | A | NT |
| 36 | A | NT |
| 37 | A | NT |
| 38 | A | NT |
| 39 | A | NT |
| 40 | A | NT |
| 41 | A | NT |
| 42 | A | NT |
| 43 | A | NT |
| 44 | A | NT |
| 45 | A | NT |
| 46 | A | NT |
| 47 | A | NT |
| 48 | A | NT |
| 49 | A | NT |
| 50 | A | NT |
| 51 | A | NT |
| 52 | A | NT |
| 53 | A | NT |

### Broken Cell Carboxylesterase Assay

Any given compound of the present invention wherein R₇ is an ester group, may be tested to determine whether it meets the requirement that it be hydrolysed by intracellular esterases, by testing in the following assay.

### Preparation of Cell Extract

U937 or HCT 116 tumour cells (˜10⁹) were washed in 4 volumes of Dulbeccos PBS (˜1 litre) and pelleted at 525 g for 10 min at 4° C. This was repeated twice and the final cell pellet was resuspended in 35 mL of cold homogenising buffer (Trizma 10 mM, NaCl 130 mM, CaCl₂ 0.5 mM pH 7.0 at 25° C.). Homogenates were prepared by nitrogen cavitation (700 psi for 50 min at 4° C.). The homogenate was kept on ice and supplemented with a cocktail of inhibitors at final concentrations of:
Leupeptin 1 μM
Aprotinin 0.1 μM
E64 8 μM
Pepstatin 1.5 μM
Bestatin 162 μM
Chymostatin 33 μM

After clarification of the cell homogenate by centrifugation at 525 g for 10 min, the resulting supernatant was used as a source of esterase activity and was stored at −80° C. until required.

### Measurement of Ester Cleavage

Hydrolysis of esters to the corresponding carboxylic acids can be measured using the cell extract, prepared as above. To this effect cell extract (˜30 μg/total assay volume of 0.5 mL) was incubated at 37° C. in a Tris-HCl 25 mM, 125 mM NaCl buffer, pH 7.5 at 25° C. At zero time the ester (substrate) was then added at a final concentration of 2.5 μM and the samples were incubated at 37° C. for the appropriate time (usually 0 or 80 min). Reactions were stopped by the addition of 3× volumes of acetonitrile. For zero time samples the acetonitrile was added prior to the ester compound. After centrifugation at 12000 g for 5 min, samples were analysed for the ester and its corresponding carboxylic acid at RT by LCMS (Sciex API 3000, HP1100 binary pump, CTC PAL). Chromatography was based on an AceCN (75×2.1 mm) column and a mobile phase of 5-95% acetonitrile in water/0.1% formic acid.

The table below presents data showing that several amino acid ester motifs, conjugated to various intracellular enzyme inhibitors by several different linker chemistries are all hydrolysed by intracellular carboxyesterases to the corresponding acid.

| | | | Hydrolysis Rate | Preparation of |
|---|---|---|---|---|
| | | | Range U937 Cells | amino ester |
| Structure of amino acid ester conjugate | R | Linker | (pg/mL/min) | conjugate |
| 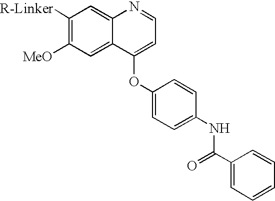 | 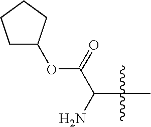 | —CH2CH2O— | 100-1000 | WO2006117552 |
| 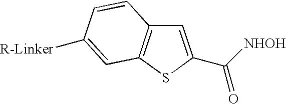 | 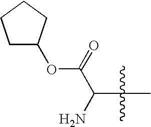 | 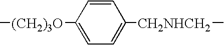 | 1000-50000 | WO2006117548 |
| 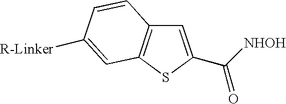 | 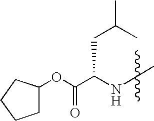 | 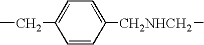 | >50000 | WO2006117549 |
| 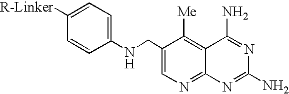 | 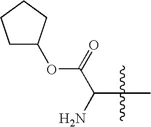 | —CH2CH2O— | >50000 | WO2006117567 |
| 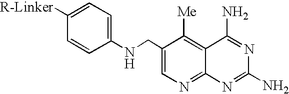 | 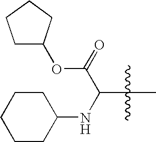 | —CH2CH2O— | 1000-50000 | WO2006117567 |
| 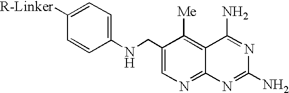 | 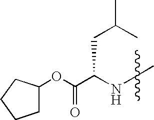 | —CH2— | 1000-50000 | WO2006117567 |
| 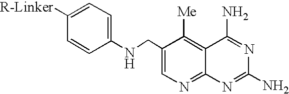 | 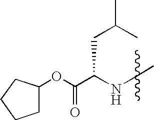 | —CO— | >50000 | WO2006117567 |
| 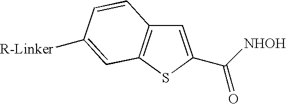 | 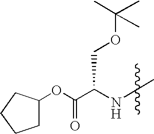 | 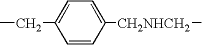 | >50000 | WO2006117549 |
| 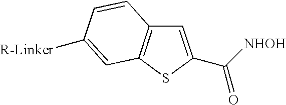 | 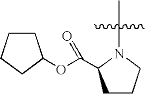 | 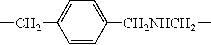 | >50000 | WO2006117549 |

The table below shows that Example 22 containing a cleavable esterase motif has much greater activity in cells than the compound lacking the esterase motif, compound I (Example 46 in WO04076454), even though both have similar enzyme activities.

| | | | | Inhibition of | |
|---|---|---|---|---|---|
| | | | | | U937 cell |
| | | Inhibition of PLK | | proliferation | |
| | | (IC50, nM) | | (IC50, nM) | Ratio |
| Compound | Structure | ester | acid | (method B) | cell/enzyme |
| Compound 1 | 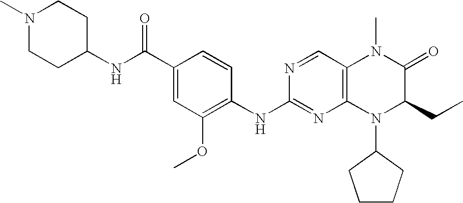 | 4 | | 1.6 | 0.4 |
| Example 22 | 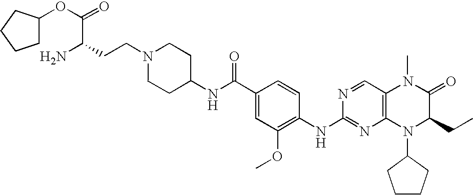 | 6 | 6 (Example 53) | 0.09 | 0.015 |

1. A compound of formula (I), or a salt, N-oxide, hydrate or solvate thereof: 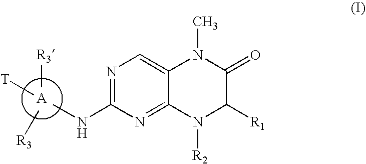
wherein
R₁ is hydrogen, or an optionally substituted (C₁-C₆)alkyl, (C₂-C₆)alkenyl, (C₂-C₆)alkynyl or (C₃-C₆)cycloalkyl group;
R₂ is hydrogen, or an optionally substituted (C₁-C₆)alkyl, (C₂-C₆)alkenyl, (C₂-C₆)alkynyl or (C₃-C₆)cycloalkyl group;
R₃ and R₃′ are independently selected from hydrogen, —CN, hydroxyl, halogen, optionally substituted (C₁-C₆)alkyl, (C₂-C₆)alkenyl, (C₂-C₆)alkynyl or (C₃-C₆)cycloalkyl, —NR₅R₆ or C₁-C₄ alkoxy, wherein R₅ and R₆ are independently hydrogen or optionally substituted (C₁-C₆)alkyl;
ring A is an optionally substituted mono- or bi-cyclic carbocyclic or heterocyclic ring or a ring system having up to 12 ring atoms;
T is a radical of formula R-L¹-Y¹— wherein
Y¹ is a bond, —O—, —S—, —NR₆—, —(C═O)—, —S(O₂)—, —(C═O)NR₆—, —NR₆(C═O)—, —S(O₂)NR₆—, —NR₆S(O₂)—, or —NR₆(C═O)NR₉—, wherein R₆ and R₉ are independently hydrogen or optionally substituted (C₁-C₆)alkyl;
L¹ is a divalent radical of formula -(Alk¹)ₘ(Q)ₙ(Alk²)ₚ- wherein m, n and p are independently 0 or 1,
Q is (i) an optionally substituted divalent mono- or bicyclic carbocyclic or heterocyclic radical having 5-13 ring members, or (ii), in the case where p is 0, a divalent radical of formula -Q¹-X²— wherein X² is —O—, —S— or NR^{A}— wherein R^{A} is hydrogen or optionally substituted C₁-C₃ alkyl, and Q¹ is an optionally substituted divalent mono- or bicyclic carbocyclic or heterocyclic radical having 5-13 ring members,
Alk¹ and Alk² independently represent optionally substituted divalent (C₃-C₆)cycloalkyl radicals, or optionally substituted straight or branched, (C₁-C₆)alkylene, (C₂-C₆)alkenylene, or (C₂-C₆)alkynylene radicals which may optionally contain or terminate in an ether (—O—), thioether (—S—) or amino (—NR^{A}—) link wherein R^{A} is hydrogen or optionally substituted (C₁-C₃)alkyl;
R is a radical of formula (X) or (Y) 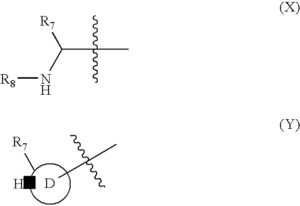
wherein
R₇ is a carboxylic acid group (—COOH), or an ester group which is hydrolysable by one or more intracellular carboxylesterase enzymes to a carboxylic acid group;
R₈ is hydrogen; or optionally substituted C₁-C₆ alkyl, C₃-C₇ cycloalkyl, aryl or heteroaryl or —(C═O)R₆, —(C═O)OR₆, or —(C═O)NR₆ wherein R₆ is hydrogen or optionally substituted (C₁-C₆)alkyl; and
D is a monocyclic heterocyclic ring of 5 or 6 ring atoms wherein R₇ is linked to a ring carbon adjacent the ring nitrogen shown, and ring D is optionally fused to a second carbocyclic or heterocyclic ring of 5 or 6 ring atoms in which case the bond shown intersected by a wavy line may be from a ring atom in said second ring.

2. A compound as claimed in claim 1 wherein R₁ is ethyl.

3. A compound as claimed in claim 1 wherein R₂ is cyclopentyl.

4. A compound as claimed in claim 1 wherein ring A is a phenyl ring.

5. A compound as claimed in claim 1 wherein R₃ and R₃′ are hydrogen, methoxy, trifluoromethoxy, —CN, hydroxyl, chloro, fluoro, methyl, trifluoromethyl, ethyl, n- and iso-propyl, allyl, —CH₂C≡CH, cyclopropyl, cyclopentyl, cyclohexyl, —NR₅R₆ wherein R₅ and R₆ are independently hydrogen, methyl or ethyl.

6. A compound as claimed in claim 1 wherein R₃ is methoxy, fluoro or chloro, and R′₃ is hydrogen, fluoro or chloro.

7. A compound as claimed in claim 1 having formula (IA): 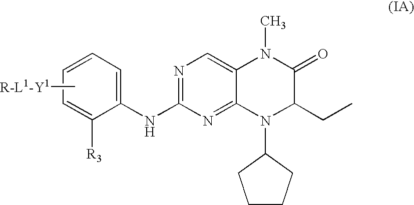
wherein R₃ is methoxy, fluoro or chloro, and the remaining variables are as defined in claim 1.

8. A compound as claimed in claim 1 wherein R₇ is of formula —(C═O)OR₁₀ wherein R₁₀ is R₁₁R₁₂R₁₃C— wherein
(i) R₁₁ is hydrogen or optionally substituted (C₁-C₃)alkyl-(Z¹)ₐ—[(C₁-C₃)alkyl]_{b}- or (C₂-C₃)alkenyl-(Z¹)ₐ—[(C₁-C₃)alkyl]_{b}- wherein a and b are independently 0 or 1 and Z¹ is —O—, —S—, or —NR₁₄— wherein R₁₄ is hydrogen or (C₁-C₃)alkyl; and R₁₂ and R₁₃ are independently hydrogen or (C₁-C₃)alkyl-;
(ii) R₁₁ is hydrogen or optionally substituted R₁₅R₁₆N—(C₁-C₃)alkyl- wherein R₁₅ is hydrogen or (C₁-C₃)alkyl and R₁₆ is hydrogen or (C₁-C₃)alkyl; or R₁₅ and R₁₆ together with the nitrogen to which they are attached form an optionally substituted monocyclic heterocyclic ring of 5- or 6-ring atoms or bicyclic heterocyclic ring system of 8 to 10 ring atoms, and R₁₂ and R₁₃ are independently hydrogen or (C₁-C₃)alkyl-; or
(iii) R₁₁ and R₁₂ taken together with the carbon to which they are attached form an optionally substituted monocyclic carbocyclic ring of from 3 to 7 ring atoms or bicyclic carbocyclic ring system of 8 to 10 ring atoms, and R₁₃ is hydrogen.

9. A compound as claimed in claim 8 wherein R₁₀ is methyl, ethyl, n- or iso-propyl, n-, sec- or tert-butyl, cyclohexyl, allyl, phenyl, benzyl, 2-, 3- or 4-pyridylmethyl, N-methylpiperidin-4-yl, tetrahydrofuran-3-yl, methoxyethyl, indanyl, norbonyl, dimethylaminoethyl, morpholinoethyl.

10. A compound as claimed in claim 8 wherein R₁₀ is cyclopentyl or tert-butyl.

11. A compound as claimed in claim 1 wherein R is a radical of formula (X) and R₈ is hydrogen.

12. A compound as claimed in claim 1 wherein, in the radical L¹, Y¹ is —NHC(═O)—.

13. A compound as claimed in claim 1 wherein, in the radical L¹, Alk¹ and Alk² radicals, when present, are selected from —CH₂—, —CH₂CH₂— —CH₂CH₂CH₂—, —CH₂CH(OH)CH₂—, —CH₂CH₂CH₂CH₂—, —CH═CH—, —CH═CHCH₂—, —CH₂CH═CH—, —CH₂CH═CHCH₂—, —C≡C—, —C≡CCH₂—, —CH₂C≡C—, CH₂C≡CCH₂—CH₂W—, —CH₂CH₂W—, —CH₂CH₂WCH₂—, —CH₂CH₂WCH(CH₃)—, —CH₂WCH₂CH₂—, —CH₂WCH₂CH₂WCH₂—, —WCH₂CH₂—, —CH₂CH₂N(CH₂CH₂OH)CH₂, and divalent cyclopropyl, cyclopentyl and cyclohexyl radicals; W being —O—, —S—, —NH—, or —N(CH₃)—.

14. A compound as claimed in claim 1 wherein, in the radical L¹, Q, when present, is a divalent phenylene, pyridinylene, pyrimidinylene, pyrazinylene, piperidinylene, piperazinylene, pyrrolidenylene, pyrrolene, cyclopropylene, cyclobutylene, cyclopentylene, cyclohexylene or 3-aza-bicyclo[3.1.0]hexylene radical

15. A compound as claimed in claim 1 wherein Q, when present, is a divalent 1,4-phenylene, 1,4-piperidinylene, or 1,4-piperazinylene radical.

16. A compound as claimed in claim 1 selected from the group consisting of:
Cyclopentyl 4-[(4-{[(7R)-8-cyclopentyl-7-ethyl-5-methyl-6-oxo-5,6,7,8-tetrahydro pteridin-2-yl]amino}-3-methoxybenzoyl)amino]-phenylalaninate,
Cyclopentyl O-(4-{[(4-{[(7R)-8-cyclopentyl-7-ethyl-5-methyl-6-oxo-5,6,7,8-tetrahydro pteridin-2-yl]amino}-3-methoxybenzoyl)amino]methyl}phenyl)-L-homoserinate,
tert-butyl 4-[(4-{[(7R)-8-cyclopentyl-7-ethyl-5-methyl-6-oxo-5,6,7,8-tetrahydropteridin-2-yl]amino}-3-methoxybenzoyl)amino]-L-phenylalaninate,
tert-Butyl O-(4-{[(4-{[(7R)-8-cyclopentyl-7-ethyl-5-methyl-6-oxo-5,6,7,8-tetrahydro pteridin-2-yl]amino}-3-methoxybenzoyl)amino]methyl}phenyl)-L-homoserinate,
Cyclopentyl 4-{2-[(4-{[(7R)-8-cyclopentyl-7-ethyl-5-methyl-6-oxo-5,6,7,8-tetrahydro pteridin-2-yl]amino}-3-methoxybenzoyl)amino]ethyl}piperazine-2-carboxylate,
tert-butyl 4-{2-[(4-{[(7R)-8-cyclopentyl-7-ethyl-5-methyl-6-oxo-5,6,7,8-tetrahydro pteridin-2-yl]amino}-3-methoxybenzoyl)amino]ethyl}piperazine-2-carboxylate,
Cyclopentyl (2S)-2-amino-4-{4-[(4-{[(7R)-8-cyclopentyl-7-ethyl-5-methyl-6-oxo-5,6,7,8-tetrahydropteridin-2-yl]amino}-3-methoxybenzoyl)amino]piperidin-1-yl}butanoate,
tert-butyl 5-{4-[(4-{[(7R)-8-cyclopentyl-7-ethyl-5-methyl-6-oxo-5,6,7,8-tetrahydro pteridin-2-yl]amino}-3-methoxybenzoyl)amino]piperidin-1-yl}-L-norvalinate,
Cyclopentyl 5-{4-[(4-{[(7R)-8-cyclo pentyl-7-ethyl-5-methyl-6-oxo-5,6,7,8-tetrahydro pteridin-2-yl]amino}-3-methoxybenzoyl)amino]piperidin-1-yl}-L-norvalinate,
t-butyl (2S)-2-amino-4-{4-[(4-{[(7R)-8-cyclopentyl-7-ethyl-5-methyl-6-oxo-5,6,7,8-tetrahydropteridin-2-yl]amino}-3-methoxy benzoyl)amino]piperidin-1-yl}butanoate,
t-butyl (2S)-2-amino-4-{4-[(4-{[(7R)-8-cyclopentyl-7-ethyl-5-methyl-6-oxo-5,6,7,8-tetrahydropteridin-2-yl]amino}-3-methylbenzoyl)amino]piperidin-1-yl}butanoate,
Cyclopentyl (2S)-2-amino-4-{4-[(4-{[(7R)-8-cyclopentyl-7-ethyl-5-methyl-6-oxo-5,6,7,8-tetrahydropteridin-2-yl]amino}-3-methylbenzoyl)amino]piperidin-1-yl}butanoate,
t-butyl (2S)-2-amino-4-{4-[(4-{[(7R)-8-cyclopentyl-7-ethyl-5-methyl-6-oxo-5,6,7,8-tetrahydropteridin-2-yl]amino}-3-fluorobenzoyl)amino]piperidin-1-yl}butanoate,
Cyclopentyl (2S)-2-amino-4-{4-[(4-{[(7R)-8-cyclopentyl-7-ethyl-5-methyl-6-oxo-5,6,7,8-tetrahydropteridin-2-yl]amino}-3-fluorobenzoyl)amino]piperidin-1-yl}butanoate,
and salts, N-oxides, hydrates or solvates thereof.

17. A pharmaceutical composition comprising a compound as claimed in claim 1, together with a pharmaceutically acceptable carrier.

18. (canceled)

19. A method of treatment of conditions mediated by PLK1 activity, which comprises administering to a subject suffering such disease an effective amount of a compound of formula (I) as claimed in claim 1.

20. The method as claimed in claim 19 for treatment of cell proliferative diseases.

21. The method as claimed in claim 19 for treatment of solid tumours.

22. The method as claimed in claim 19 for treatment of haemato-oncological tumours.
